(19) United States
(12) Patent Application Publication
(10) Pub. No.: US 2024/0087678 A1
(43) Pub. Date: Mar. 14, 2024

(54) Cellular Analysis with Topology and Condensation Homology (CATCH) Analysis and Method of Use

(71) Applicant: Yale University, New Haven, CT (US)

(72) Inventors: Manik Kuchroo, New Haven, CT (US); Smita Krishnaswamy, New Haven, CT (US)

(21) Appl. No.: 18/465,301

(22) Filed: Sep. 12, 2023

Related U.S. Application Data
(60) Provisional application No. 63/375,304, filed on Sep. 12, 2022.

(51) Int. Cl. G16B 20/00; G16B 40/20; G16H 50/20

(52) U.S. Cl. CPC G16B 20/00; G16B 40/20; G16H 50/20

(57) ABSTRACT

The present invention describes a CATCH assay for detecting cellular populations, biomarkers or biological interactions in a sample, and methods of use of the assay for identifying novel biomarkers of diseases and disorders and for diagnosing or treating diseases and disorders.

### CROSS REFERENCE TO RELATED APPLICATIONS

This application claims priority to U.S. Provisional Application No. 63/375,304, filed Sep. 12, 2022 which is hereby incorporated by reference herein in its entirety.

### STATEMENT REGARDING FEDERALLY SPONSORED RESEARCH OR DEVELOPMENT

This invention was made with government support under AI157270 and GM135929 awarded by National Institutes of Health and under 2047856 awarded by the National Science Foundation. The government has certain rights in the invention.

### BACKGROUND OF THE INVENTION

Cells can exist in various transcriptional states, which naturally fall into a hierarchy or organization. Within this hierarchy, cells of a more similar functional niche, for instance microglia and astrocytes, are more closely related to one another than cells of a more disparate niche, for instance microglia and endothelial cells. Learning this hierarchy from data is critical to the development of a systematic understanding of biological function and can provide insight into mechanisms of disease pathogenesis. As cell types may be differentially affected by disease, the simultaneous identification and characterization of abundant classes of cells at coarse granularity as well as rare cell types or states at fine granularity provides a comprehensive framework for defining, modeling, and understanding specific cellular pathways in disease. While advances in high throughput single cell protocols capture vast amounts of heterogeneity (Habib, N. et al., 2017, Nature Methods 14, 955-958) allowing for the comparison of diseased and healthy tissue, no computational tools currently exist that systematically sweep through all possible granularities of the cellular hierarchy to identify pathogenic populations, and infer disease-specific mechanisms.

The key to thoroughly identifying and characterizing populations of cells affected by disease across granularities lies in the accurate computation of the cellular hierarchy. Current hierarchical clustering approaches applied to single cell analysis enforce global granularity constraints and provide only a few salient levels at which cellular groups can be found (Blondel, V. D. et al., 2008, Journal of Statistical Mechanics: Theory and Experiment 2008, P10008; Traag, V. A. et al., 2019, Scientific Reports 9). This not only limits the discovery of rare disease-associated populations, but also requires computationally expensive differential expression analysis tools that produce diluted signatures of disease from unrefined clusters of cells.

There is thus a need in the art for accurate methods for detecting disease signatures from rare disease-associated populations. The present invention addresses this unmet need in the art.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of several embodiments of the invention will be better understood when read in conjunction with the appended drawings. For the purpose of illustrating the invention, there are shown in the drawings embodiments which are exemplified. It should be understood, however, that the invention is not limited to the precise arrangements and instrumentalities of the embodiments shown in the drawings.

FIG. 1A through FIG. 1D depict a comparison of diffusion condensation against differing implementations and other clustering approaches on synthetic and real single cell data. FIG. 1A depicts a box and whisker plot comparing CATCH, Louvain, Leiden and FlowSOM on flow cytometry data with cluster labels have been identified through conventional gating analysis. Comparison was repeated on 1.3 million cells generated from 30 patients in the FlowCAP dataset. FIG. 1B depicts a comparison of CATCH against multigranular clustering approaches, Louvain and Leiden, on real single cell data across clustering granularity. FIG. 1C depicts a graph showing diffusion condensation cluster characterization implemented with α-decay and Gaussian kernels compared to ground truth cluster characterizations across granularities on 4,360 PBMCs measured with 10×. FIG. 1D depicts a graph comparing correlation between ground truth EMD values with condensed transport values across granularities using the same multi-cluster and multi-granular comparison strategy described in 2F on 10× single-cell data. Reported correlation values as a feature of cluster granularity (denoted by number of cluster on x-axis) for both Gaussian and α-decay kernels, representing 12,061,332 and 3,373,476 comparisons respectively.

FIG. 2A through FIG. 2F depict key advancements in CATCH clustering approach. FIG. 2A depicts a graph showing the ideal number of t-steps calculated by spectral entropy per iteration when running diffusion condensation on 4,360 single cell PBMCs measured on the 10× platform. FIG. 2B depicts graphs comparing different CATCH implementations on synthetic splatter data with increasing levels of noise. Comparing implementations with fixed numbers of t steps set for every iteration (t=1,2,3) against the final diffusion condensation approach which uses spectral entropy to tune t at every iteration. Adaptively tune t outperforms other implementations significantly as noise levels increase across noise types (two-sided Student's ttest, p<0.05). FIG. 2C depicts a visualization of difference between Gaussian and α-decay kernels. FIG. 2D depicts a graph comparing differentially expressed genes using CATCH condensed transport implemented with Gaussian kernel and α-decay kernel against ground truth 1D-Wasserstein EMD distance. In each comparison, CATCH was run on 10,000 cells generated from splatter as done in part B. Topological activity analysis was used to compute salient granularities for downstream analysis. In each comparison, all salient granularities with less than 20 clusters was used. At each granularity, differentially expressed genes were computed between every combination of clusters. Across all comparisons, 10,249,140 and 4,535,640 comparisons for the Gaussian and α-decay kernel implementations were computed respectively. FIG. 2E depicts a graph comparing correlation between ground truth EMD values with condensed transport values across granularities using the same multicluster and multi-granular comparison strategy described in FIG. 2D. Visualizing reported correlation values as a feature of cluster granularity (denoted by number of cluster on x-axis) for both Gaussian and α-decay kernels. FIG. 2F depicts a run time comparison between scalable and standard implementations of CATCH.

FIG. 3A through FIG. 3F depict data demonstrating that diffusion condensation identifies and characterizes populations of related cells across granularities. FIG. 3A depicts a PHATE visualization of 10,000 cells generated from splatter (Zappia, L et al., 2017, Genome Biology 18). Noiseless data is first generated on which ground truth clusters are computed (left). Two types of biological noise, variation and drop out, are simulated and the dataset is revisualized with ground truth cluster labels highlighted (right). FIG. 3B depicts a condensation homology visualization of noisy splatter single cell data simulation. Four granularities are highlighted (represented as i.-iv.), illustrating 4 resolutions identified in FIG. 3C as meaningful. FIG. 3C depicts graphs where topological activity is first computed (left) before gradient analysis is performed on the topological activity curve (right). Resolutions identified by this analysis as being meaningful are highlighted (represented as i.-iv.). FIG. 3D depicts visualizations showing meaningful resolutions identified in FIG. 3C which are represented with Adjusted Rand Index score when compared to ground truth cluster labels shown. Visualizations are arranged from the coarsest granularity of clusters (left) to the finest granularity (right). FIG. 3E depicts graphs where forty different splatter synthetic single cell datasets are simulated with either increasing amounts of drop out (left panel) or biological variation (right panel). Cluster labels are computed using a range of multiresolution clustering techniques (CATCH, Louvain, Leiden, Seurat). The top four most optimal resolutions from each algorithm are compared to ground truth cluster labels computed on noiseless data, with the highest Adjusted Rand Index score saved. This is repeated 10 times using different random seeds for each algorithm. Shading represents 2 standard deviations around the mean of each algorithm's performance. At the highest levels of dropout and variational noise, CATCH performs significantly better than Louvain, Leiden and Seurat (p<0.05, two-sided Student's t-test, with multiple comparisons testing). FIG. 3F depicts a graph showing condensed transport with an α-decay kernel shows superior fidelity with ground truth Earth Mover's Distance on 4,360 single cell PBMCs measured on the 10× platform. The diffusion condensation algorithm was implemented with α-decay (left panel) and Gaussian (right panel) kernels. In each implementation, topological activity analysis identified persistent clustering granularities. For this comparison, granularities with 20 or less clusters were analyzed by comparing all clusters to each other. This resulted in U.S. Pat. Nos. 10,166,640 and 2,541,660 comparisons for the α-decay kernel and Gaussian implementations respectively.

FIG. 4A through FIG. 4D depict an overview of neurodegenerative disease processes and the topological diffusion condensation approach. FIG. 4A depicts a sketch of retina cross section showing layers and major cell types. FIG. 4B is an illustration of the role of innate immune cells in neurodegenerative disease pathogenesis. In the dry stage of AMD, there is accumulation of extracellular drusen debris between Bruch's membrane (BM) and the retinal pigment epithelium (RPE), leading to activation of glia. Accumulation of extracellular plaques and intracellular neurofibrillary tangles in Alzheimer's disease and myelin damage in progressive multiple sclerosis are both accompanied by microglia (blue) and astrocyte (orange) activation. FIG. 4C depicts a visual description of cellular condensation process undertaken by diffusion condensation across 4 granularities. Points are iteratively moved to and merged with their nearest neighbors as determined by a weighted random walk over the data graph. Over many successive iterations, cells collapse, denoting cluster identity at various iterations. FIG. 4D depicts the coarse graining process described in (FIG. 4C) creates hundreds of granularities of clusters which can be analyzed in meaningful ways: i) the condensation homology, or hierarchy of clusters computed by diffusion condensation can be visualized, to identify the merging behavior across granularities; ii) meaningful, persistent partitions of the data can be identified by performing topological activity analysis; iii) in conjunction with MELD (Burkhardt, D. B. et al., 2020, bioRxiv), these meaningful granularities can be scanned across to identify resolutions that optimally split disease-enriched populations of cells from healthy populations of cells and finally iv) top most differentially enriched genes between populations of interested can be computed using condensed transport.

FIG. 5A through FIG. 5E depict data demonstrating single-nucleus RNA-seq profiling of the macula from human individuals with varying stages of AMD pathology. FIG. 5A (left) depicts topological activity analysis of human retina single-cell data across all condensation iterations. By computing gradients on topological activity, three granularities at which persistent partitions of the data occur were identified (represented by resolutions i, ii and iii), and selected for downstream analysis. (right) Condensation process of AMD single-cell data visualized across iterations (from bottom to top) with the most coarse-grained granularity clusters visualized on PHATE embedding: resolution i. represents the most coarse-grained clusters and resolution ii. represents the second most coarse-grained clusters. FIG. 5B depicts populations identified at the finest granularity identified by topological activity analysis (resolution iii.) were visualized and all populations were assigned a cell type based on which cell-type gene signature they displayed the highest expression. FIG. 5C depicts cell-type-specific genes visualized along with average normalized expression of known cell-type-specific marker genes. All major retinal cell types were identified by CATCH process described in FIG. 5A, FIG. 5B. FIG. 5D depicts differentially expressed genes identified by Wasserstein Earth Mover's Distance (EMD) between cells from early-stage dry and late-stage neovascular AMD lesions and cells from control retinas on a cell-type-specific basis. Number of significantly differentially expressed genes between control and AMD cells reported in a cell type and stage-specific manner (FDR corrected p-value <0.1). Cell types sorted by most differential genes between dry AMD and control comparison. Vascular cells, microglia and astrocytes have the most differentially expressed genes in dry AMD compared to control samples. FIG. 5E depicts a bar chart that indicates the contribution of cell types in each cluster from control, dry AMD and neovascular AMD samples. Microglia and astrocytes are the most statistically significantly enriched cell types in AMD, while rods and cones are the most depleted cell types in neovascular AMD. Vascular cells are the most enriched cell type in the neovascular AMD condition. All statistics were computed using two-sided multinomial tests with multiple comparisons correction (*p<0.1).

FIG. 6A through FIG. 6E depict data demonstrating that CATCH identifies known subtypes of bipolar cells across multiple levels of granularity. FIG. 6A depicts a visualization of cell type specific signatures based on composite normalized expression of cell type specific marker genes visualized on PHATE. FIG. 6B depicts an analysis showing that CATCH identifies ON and OFF bipolar cell subsets at a granularity identified with topological activity analysis. FIG. 6C depicts finer grained analysis of ON bipolar cells reveals known subsets. FIG. 6D depicts finer grained analysis of OFF bipolar cells reveals known subsets. FIG. 6E depicts a heat map showing that CATCH reliably identifies established cell types, as shown by average normalized expression of known bipolar subset-specific marker genes.

FIG. 7A and FIG. 7B depict data demonstrating that Louvain does not identify rare glial populations across granularities. FIG. 7A depicts a visualization of 22 coarse grain clusters identified by Louvain. The identified populations are not able to identify all known cell types, as shown by the average normalized expression of known cell type specific marker genes. FIG. 7B depicts a visualization of 40 fine grain clusters identified by Louvain. The identified populations at this granularity also do not resolve all known cell types, as shown by the average normalized expression of known cell type specific marker genes.

FIG. 8A through FIG. 8G depict data demonstrating that fine grain analysis of microglia reveals a shared activation signature enriched in the early phase of three different neurodegenerative diseases. FIG. 8A depicts a visualization of one hundred and forty one microglia identified by diffusion condensation at coarse granularity (upper left) can be further subdivided into three clusters at fine granularity, each enriched for cells from a different disease-state. Disease state enrichment was calculated using MELD (right) for each condition: Control (top), Dry AMD (middle) and Neovascular AMD (bottom), with higher MELD likelihoods shown with darker colors. A resolution of the condensation homology which optimally isolated MELD likelihood scores from each condition was identified using topological activity analysis. Microglia are revisualized using PHATE. FIG. 8B depicts a visualization as in FIG. 8A, where three subsets of 288 microglia are found in AD with diffusion condensation and topological activity analysis, each enriched for cells from a different disease condition as computed by MELD (right). Cells are revisualized with PHATE. FIG. 8C depicts a visualization as in FIG. 8A, where three subsets of 1263 microglia are found in MS with diffusion condensation and topological activity analysis, each enriched for cells from a different disease condition as computed by MELD (right). Cells are revisualized with PHATE. FIG. 8D depicts a differential expression analysis as computed by condensed transport between healthy-enriched and early or acute active disease-enriched across all three degenerative diseases reveals a shared activation pattern in early disease (increased expression of TYROBP, B2M, APOE, CD74, SPP1, HLA-DR, C1QB, C1QC). Significance values (dark grey) were assigned as top 10% of condensed transport values. FIG. 8E depicts a heatmap demonstrating differences in expression of the neurodegenerative shared activation pattern and a homeostatic signature between healthy-enriched and early or acute active disease-enriched across all three degenerative diseases. Color conventions are as in FIG. 8A-C. FIG. 8F, upper depicts a graph of composite microglial activation signature for the neurodegenerative shared activation pattern in healthy-enriched and early or acute active disease-enriched across all three degenerative diseases (y-axis—gene expression of signature). (F, lower) Disease associated microglia (DAM) signature (from (Keren-Shaul, H. et al., 2017, Cell 169, 1276-1290)) for healthy-enriched and early or acute active disease-enriched across all three degenerative diseases. Color conventions are as in panels A-C. (y-axis—gene expression of signature) FIG. 8G depicts micrographs of combined in-situ RNA hybridization and IBA1 immunofluorescence demonstrating elevated expression of key components of the neurodegenerative shared activation pattern (TYROBP and APOE) in IBA1-positive cells, a marker of microglia, from retinas with dry AMD (right group) compared to healthy controls (left group). All scale bars=10 μm. The average number of puncta identified per IBA1-positive cell for TYROBP was 0.28±0.05 in dry AMD vs. 0.02±0.01 for control (p<1e-10; Chi-square test for 0 vs. >0). The average number of puncta identified per IBA1-positive cell for APOE was 0.57±0.09 in dry AMD vs. 0.14±0.03 for control (p<1e-08; Chi-square test for 0 vs. >0).

FIG. 9A through FIG. 9C depict data demonstrating the control probes for fluorescence in situ hybridization and validation of early activation microglial signature in MS and AD. FIG. 9A depicts representative images of fluorescence in situ hybridization for positive control probe (POL2RA labeled in red and UBC labeled in yellow). FIG. 9B depicts representative images of fluorescence in situ hybridization for negative control probe (DapB labeled in yellow and red). FIG. 9C depicts representative images of in situ RNA hybridization of APOE (labeled in turquoise), TYROBP (labeled in pink) with simultaneous immunofluorescence of microglial marker IBA1 (white). Elevated expression of APOE and TYROBP is seen in IBA1-positive cells in microglia from brain tissue with early AD and early progressive MS, compared to healthy controls. Each row represents a sample from a different case. All scale bars=10 μm.

FIG. 10A through FIG. 10F depict data demonstrating that CATCH analysis of AD and MS snRNAseq data reveals enrichment and activation of microglia and astrocytes in disease. FIG. 10A depicts an analysis of 43,650 cells pooled from 48 AD patients and healthy donors. Samples were taken from disease free brain tissue and diseased brain tissue at early and late pathological stages. All major cell types were identified by CATCH via persistence analysis and visualized with PHATE (Moon, K. R. et al., 2019, Nature Biotechnology 37, 1482-1492). Ideal CATCH granularity identified via topological activity analysis (right). FIG. 10B depicts an analysis of 46,796 cells pooled from 21 progressive MS patients and healthy donors. Samples were taken from disease free brain tissue and diseased brain tissue at acute and chronic stages of inflammation. All major cell types were identified by CATCH via persistence analysis and visualized with PHATE (Moon, K. R. et al., 2019, Nature Biotechnology 37, 1482-1492). Ideal CATCH granularity identified via topological activity analysis (right). FIG. 10C depicts a heatmap showing that CATCH reliably identifies cell types in AD brain tissue, as shown by average normalized expression of known cell type-specific marker genes. FIG. 10D depicts a heatmap showing that CATCH reliably identifies cell types in MS brain tissue, as shown by average normalized expression of known cell type-specific marker genes. FIG. 10E depicts a graph showing that microglia and astrocytes are the most enriched cell types in AD using cross condition abundance analysis. FIG. 10F depicts a graph showing that microglia and astrocytes are significantly enriched in progressive MS using cross condition abundance analysis. In FIG. 10E,F: *=p<0.01, two-sided multinomial test with multi-test correction.

FIG. 11A through FIG. 11H depict data demonstrating that fine grain analysis of astrocytes reveals a shared activation signature enriched in the early phase of three different neurodegenerative diseases. FIG. 11A depicts a visualization of four hundred and seventy four astrocytes identified by diffusion condensation at coarse granularity (upper left) can be further subdivided into three clusters at fine granularity, each enriched for cells from a different disease-state. Disease state enrichment was calculated using MELD (right) for each condition: Control (top), Dry AMD (middle) and Neovascular AMD (bottom), with higher MELD likelihoods shown with darker colors. A resolution of the condensation homology which optimally isolated MELD likelihood scores from each condition was identified using topological activity analysis. astrocytes are revisualized using PHATE. FIG. 11B depicts a visualization of as in FIG. 11A where three subsets of 2361 astrocytes are found in AD with diffusion condensation and topological activity analysis, each enriched for cells from a different disease condition as computed by MELD (right). Cells are revisualized with PHATE. FIG. 11C depicts a visualization of as in FIG. 11A where three subsets of 5469 astrocytes are found in MS with diffusion condensation and topological activity analysis, each enriched for cells from a different disease condition as computed by MELD (right). Cells are revisualized with PHATE. FIG. 11D depicts a differential expression analysis as computed by condensed transport of genes between control-enriched and early disease-enriched clusters across all three neurodegenerative diseases reveals a shared activation pattern in the early stage of disease. This signature includes B2M, CRYAB, VIM, GFAP, AQP4, APOE, ITM2B, CD81, FTL. Significance values (dark grey) were assigned as top 10 percent of condensed transport values. FIG. 11E depicts a heatmap demonstrating differences in astrocyte expression of the neurodegenerative shared activation pattern and a homeostatic signature between cluster 1 and cluster 2 across all three degenerative diseases. Color conventions are as in FIG. 11A-C. FIG. 11F depicts a heatmap of composite astrocyte activation signature for the neurodegenerative shared activation pattern in control enriched cluster and early disease-enriched cluster across all three degenerative diseases. Color conventions are as in FIG. 11A-C. (y-axis—gene expression of signature). FIG. 11G depicts a micrographs of combined in-situ RNA hybridization and GFAP immunofluorescence showing more abundant B2M expression in astrocyte-rich retinal layers from dry AMD retina when compared to control. All scale bars=10 μm. FIG. 11H depicts a bar plot showing density of B2M transcripts in the astrocyte-rich inner plexiform layer, retinal ganglion cell layer, and nerve fiber layer in retina samples affected by dry AMD and control.

FIG. 12A and FIG. 12B depict data demonstrating the cell type level differential expression analysis at coarse granularity across neurodegenerative diseases. FIG. 12A depicts a graph showing that performing EMD-based differential expression analysis between microglia which originate from dry AMD patients and control subjects identified a gene signature enriched in the early stage of dry AMD. By performing similar differential expression analysis between microglia from brain samples from patients with early AD, acute active MS, and controls, a shared activation signature of 17 genes were identified. This common signature includes APOE, SPP1 and B2M while all other genes are highlighted in red. FIG. 12B depicts a graph showing that performing EMD-based differential expression analysis between astrocytes which originate from dry AMD patients and control subjects identified a gene signature enriched in the early stage of dry AMD. By performing similar differential expression analysis between astrocytes from control and early AD samples and control and acute inflammation MS samples, a shared activation signature of 28 genes were identified. This common signature includes GFAP, AQP4, and CRYAB while all other genes are highlighted in red.

FIG. 13A through FIG. 13C depict data demonstrating that the shared activation signatures is diminished in advanced disease and replaced with disease-specific stress signature in microglia. FIG. 13A depicts data comparing advanced or chronic inactive disease-enriched microglial cluster to early or acute active enriched microglial cluster reveals a significant reduction in the microglial activation signature across later stages neurodegeneration. FIG. 13B depicts data comparing advanced or chronic inactive disease-enriched astrocyte cluster to early or acute active-enriched cluster reveals a significant reduction in the astrocyte activation signature across later stages neurodegeneration. FIG. 13C depicts data comparing advanced or chronic inactive disease-enriched microglia clusters to early or acute active-enriched clusters in AD and MS does not reveal shared inflammasome signature found in neovascular AMD microglia. Early activation signature is replaced with disease-specific microglial cell stress pathway signatures.

FIG. 14A through FIG. 14F depict data demonstrating that CATCH analysis of snRNAseq data from late stage AMD tissue identifies differing activation signatures in microglia and astrocytes. FIG. 14A depicts PHATE visualization of nuclei isolated from neovascular AMD and control retinas (Moon, K. R. et al., 2019, Nature Biotechnology 37, 1482-1492). CATCH identified a resolution of the condensation homology which isolated cell types. As in FIG. 4, each cellular cluster was assigned a cell type identity based on which gene signature it expressed at the highest level. FIG. 14B depicts a heatmap showing that CATCH identified cell types, as shown by the average normalized expression of known cell type specific marker genes. FIG. 14C depicts results where disease state enrichment was calculated using MELD (right) for each condition: Control (top), and Neovascular AMD (bottom), with higher MELD likelihoods shown with darker colors. A resolution of the condensation homology which optimally isolated MELD likelihood scores from each condition was identified using topological activity analysis. Microglia are revisualized using PHATE. Two subsets of microglial cells, one enriched for microglia from neovascular retinas and another from control retinas. FIG. 14D depicts a graph showing that condensed transport of genes between the control-enriched and neovascular disease-enriched microglial clusters reveals a different activation pattern in late disease. Significance values (dark grey) were assigned as top 10 percent of condensed transport values. This signature includes NFKBIB, IL1B, NOD2, FLT1, HSP90B1, RIPK2, NFKB1, HSP90AA1, HIF1A, BCL2L1, P2RX7, TAB2, HSP90AB1. FIG. 14E depicts an analysis where disease state enrichment was calculated using MELD (right) for each condition: Control (top) and Neovascular AMD (bottom), with higher MELD likelihoods shown with darker colors. A resolution of the condensation homology which optimally isolated MELD likelihood scores from each condition was identified using topological activity analysis. Astrocytes are revisualized using PHATE. CATCH identified three subsets of astrocyte cells, one enriched for astrocytes from neovascular retinas, another from control retinas and a third equally split between conditions. FIG. 14F depicts an analysis where condensed transport of genes between the control-enriched and neovascular disease-enriched astrocyte clusters reveals a different activation pattern in late disease. Significance values (dark grey) were assigned as top 10 percent of condensed transport between distributions of expression between conditions. This signature includes NR2E1, EPAS1, VEGFA, HIF1A, HIF3A.

FIG. 15A through FIG. 15H depict data identifying cytokine regulators of astrocyte VEGFA secretion. FIG. 15A depicts an interaction analysis between diffusion condensation that identified subtypes of astrocytes and neovascular-enriched microglia (detailed in FIG. 13) computed with CellPhoneDB (Efremova, M et al., 2020, Nature Protocols 15, 1484-1506). Interactions between cytokines produced from neovascular-enriched microglia were computed against cytokine-receptors on astrocyte subtypes. Interactions between specific cytokine-receptor pairs were added to produce a single cytokine interaction value for control and neovascular astrocyte subtypes. FIG. 15B depicts a DREMI association analysis between astrocyte VEGFA expression, IL1β signaling score, and IL4 signaling score. Signaling scores for IL1β and IL4 were computing by adding receptor expression of IL1β and IL4 respectively neovascular-enriched astrocytes from FIG. 13. FIG. 15C depicts results from an experiment designed to identify positive and negative regulators of VEGFA, a human iPSC derived astrocyte in vitro system was used. First, a pool of cytokines with cytokines identified through single cell RNA-seq data was created. For experimental conditions, one cytokine of interest was subtracted from the pool to test for the necessity of each cytokine in being a positive or negative regulator of VEGFA production (y axis). Then, single cytokine stimulation was used to test the sufficiency of each cytokine to stimulate astrocyte VEGFA production (x axis). FIG. 15D depicts representative immunofluorescent images where IL-1β or PBS was injected intravitreally into a mouse eye. Retinas were collected 72 hours later for immunofluorescent imaging. FIG. 15E depicts zoomed in images of regions indicated in FIG. 15E. FIG. 15F depicts the quantification of mean fluorescence intensity (MFI) of VEGFA after injection of IL-1β or PBS in the mouse eyes after 72 hours (left) and quantification of amount of VEGFA and GFAP overlap in the ganglion cell layer of the mouse retina after injection of IL-1β or PBS (right). FIG. 15G depicts immunofluorescence imaging of human post-mortem control and neovascular AMD retinas. FIG. 15H depicts quantification of IL-1β intensity in the ganglion cell layer (GCL) over the outer nuclear layer (ONL) of the retina from FIG. 15F.

FIG. 16 depicts a table detailing human retinal specimens.

FIG. 17 depicts an illustrative computer architecture for a computer 1600 for practicing the various embodiments of the invention.

### DETAILED DESCRIPTION

The present invention relates generally to the development of a suite of analysis tools, termed CATCH, which is able to sweep across multiple levels of granularity in biological sequencing data to identify subpopulations of cells enriched in disease settings and robustly characterize them. The invention allows for the identification of specific pathogenic or disease associated cellular populations and allows for the creation of rich and accurate disease signatures. These signatures serve as a prioritized list of possible therapeutic targets.

In some embodiments, the invention relates to methods of use of the CATCH analysis toolkit to diagnose, treat, or monitor the treatment of a disease or disorder.

The present invention also relates, in part, to methods of inhibiting IL-1β for the treatment of neovascular AMD.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The terms “biomarker” and “marker” are used herein interchangeably. They refer to a substance that is a distinctive indicator of a biological process, biological event and/or pathologic condition, disease or disorder.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

The term “detecting” or “detection,” means assessing the presence, absence, quantity or amount of a given substance (e.g., a biomarker) within a sample, including the derivation of qualitative or quantitative levels of such substances.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient. The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) an associated disease or condition including alleviating symptoms of such diseases.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Systems and methods disclosed herein relate to improved methods for comparing, evaluating and ranking cellular populations or subsets thereof. The invention provides a group of topologically inspired machine learning tools to identify, characterize and compare populations of cells across the cellular hierarchy. This framework is centered around an improved diffusion condensation process, in combination with tools for condensation homology visualization, topological activity analysis, automated cluster characterization and condensed transport.

In some embodiments, topology-inspired suite of machine learning tools for single-cell analysis, ‘CATCH’ sweeps through all levels of granularity to identify, characterize, and compare pathogenic populations of cells across the hierarchy. Building on previous work in data diffusion, graph filters, topological data analysis, and optimal transport, CATCH rapidly learns the geometry of the single cell manifold and identifies populations of related cells across granularities by iteratively applying diffusion filters to the data. Mathematically related to concepts in optimal transport, the disclosed approach is empirically related to continuously performing differential expression analysis across granularities, rapidly allowing for characterization and comparison of populations of interest. This approach is particularly effective at identifying and characterizing rare populations of pathogenic cells that other approaches fail to recognize.

In the following sections, a description of each aspect of CATCH is provided. This includes a description of the topological data analysis, data diffusion, and diffusion filters as well as detailed descriptions of the diffusion condensation process.

Topological Data Analysis

Topological data analysis aims to combine geometric and topological perspectives into a single framework. The underlying idea is that geometry is useful if one is interested in precise measurements and notions of distance between objects, whereas topology is useful if one is interested in describing the relationships between objects. A hybrid perspective can be appealing in situations where the individual coordinates of data points are less important than their overall agglomeration behavior.

Persistent homology refers to a specific topological data analysis framework that is well-equipped to handle geometric data at different granularities. Originally meant to analyze distance functions on sampled manifolds, persistent homology works by approximating the manifold that is underlying a dataset X. This is achieved by constructing a simplicial complex (Vietoris, L. et al., 1927, Mathematische Annalen 97, 454-472), i.e., a generalization of a graph, containing higher-dimensional structural elements called simplices, which are subsets of the data points. For a distance threshold δ, such a simplicial complex consists of all simplices—subsets of points—whose pairwise distances are less than or equal to δ, i.e.,

[symbol]

_{δ}(X):={σ⊆X|d( [symbol] ᵢ, [symbol] ⱼ)≤δ}. (Equation 1)

A subset σ of cardinality k−1 is referred to as a k-simplex. Thus, the 0-simplices are the vertices of V_{δ}(X), the 1-simplices are the edges, and so on. For k≥1, a k-simplex σ∈V_{δ}(X) is assigned a weight w_{σ} based on the distance of its vertices so that ω_{σ}:= [symbol] D( [symbol] ᵢ, [symbol] ⱼ) The simplicial complex V_{δ}(X) serves as a backbone of the dataset X, combining a geometrical perspective (imbued via δ) with a topological one: the weighted simplices serve to describe topological features such as connected components (0D), cycles (1D), and voids (2D) in X, thus constituting a hybrid between a purely geometrical approach (focusing only on points) and purely topological one (focusing only on connectivity without incorporating distance information). Persistent homology characterizes the evolution of topological features at multiple granularities, as determined by δ. For instance, if δ is sufficiently large, all points are connected to each other, whereas for δ≈0, there will be virtually no connections. Topological features can be efficiently tracked over all potential values of δ, and each feature is assigned a persistence, a quantity that indicates over which granularities (values of δ) a feature is present. For example, if X is a densely-sampled square, it will intrinsically have one connected component, which will be assigned a high persistence. The advantage of such a description is that it is independent of the dimensionality of an input dataset, relying solely on pairwise distances. Information about the persistence of features is collected in topological descriptors, such as persistence diagrams or persistence barcodes (Cohen-Steiner, D et al., 2007, Discrete & Computational Geometry 37, 103-120; Rieck, B. et al., 2020, Topological Methods in Data Analysis and Visualization V, 87-101). In a persistence barcode, each topological feature is represented as a horizontal bar whose length indicates its persistence. Topological features that persist longer (i.e., have longer bars) are considered to be more unique and prominent, while features that persist for fewer iterations are considered ubiquitous or even noisy. The barcode metaphor yields a powerful visualization, which serves as an informative summary of a dataset's underlying features. Persistent homology has received a large degree of attention by the machine learning community due to its robustness and multi-scale properties (Hensel, F et al., 2021, Frontiers in Artificial Intelligence 4). However, until now, neither topological frameworks nor topological concepts such as persistent homology have been employed in the context of single cell analysis.

Multigranular Data Abstraction

Previous attempts at providing multigranular data abstraction of single cell data rely on hierarchical clustering, a family of methods that attempts to derive a tree of clusters based on either recursive splitting or agglomeration of data points. Splitting based approaches, including recursive bisection and divisive analysis clustering, work in an iterative, top-down fashion, each time optimizing a partition of the data into clusters (Dasgupta, A et al., 2006, In Lecture Notes in Computer Science, 256-267; Kaufman, L. et al., 2009, John Wiley & Sons vol. 344).

Agglomerative methods meanwhile, including the popular linkage clustering, or community detection methods such as Louvain (Blondel, V. D. et al., 2008, Journal of Statistical Mechanics: Theory and Experiment 2008, P10008), work in a bottom-up fashion recursively merging points into clusters. While intuitively more related to the merges in diffusion condensation, there is a fundamental difference between the coarse graining operation applied here and the greedy agglomeration approaches: hierarchical methods recursively force merges or splits in data. Although a hierarchy of cells is created through this approach, concepts of data-driven topology such as persistence calculations cannot readily be applied. Since the merging of points is arbitrary and not scaled by differences between underlying cells, the persistence of a population largely does not have meaning, creating an incomplete understanding of the topology of the data.

Inspired by concepts arising from persistent homology and the dearth of current computational techniques amenable to such analysis, the method of the invention uses a topologically-inspired approach to understand the multigranular structure of single cells based on their inherent manifold geometry.

High dimensional data can often be modeled as originating from a sampling Z={zᵢ}ᵢ₌₁^{N}⊂ [symbol] ^{d} of a d-dimensional manifold d that is mapped to observations of dimension n d, collected in X={x₁, . . . , x_{N}}⊂ [symbol] ⁿ via a nonlinear function xᵢ=ƒ(zᵢ). Intuitively, the reason for this phenomenon is that data collection measurements (modeled here via ƒ) typically result in high dimensional observations, even when the intrinsic dimensionality, or degrees of freedom, in the data is relatively low. This manifold assumption is at the core of the vast field of manifold learning (e.g., (Coifman, R. R. et al., 2006, Applied and computational harmonic analysis 21, 5-30; Moon, K. R. et al., 2018, Current Opinion in Systems Biology 7, 36-46; Van Der Maaten, L. et al., 2009, J Mach Learn Res 10, 66-71; Izenman, A. J. 2012, Wiley Interdisciplinary Reviews: Computational Statistics 4, 439-446; and references therein), which leverages the intrinsic data geometry, modeled as a manifold, for exploring and understanding patterns, trends, and structure in data.

In (Coifman, R. R. et al., 2006, Applied and computational harmonic analysis 21, 5-30), diffusion maps were proposed as a robust way to capture intrinsic manifold geometry in data using random walks that aggregate local affinity to reveal nonlinear relations in data and allow their embedding in low dimensional coordinates. These local affinities are commonly constructed using a Gaussian kernel function:

$$K ( {x}_{i} , {x}_{j} ) = exp - \frac{{‖ {x}_{i} - {x}_{j} ‖}^{2}}{ε} \qquad ( Equation 2 )$$

for i,j∈{1, . . . , N}, where K is an N N Gram matrix whose (i, j) entry is denoted by K(xᵢ, xⱼ) to emphasize the dependency on the data X. The bandwidth parameter E controls neighborhood sizes. A diffusion operator is defined as the row-stochastic matrix P=D⁻¹K where D is a diagonal matrix with D(xᵢ, xᵢ)=j K(xᵢ, xⱼ), which is referred to as the degree of xᵢ. The matrix P defines single-step transition probabilities for a time homogeneous diffusion process (which is a Markovian random walk) over the data, and is thus referred to as the diffusion operator. Furthermore, as shown in (Coifman, R. R. et al., 2006, Applied and computational harmonic analysis 21, 5-30), powers of this matrix P^{t}, for t>0, can be used for multiscale organization of X, which can be interpreted geometrically when the manifold assumption is satisfied. While originally conceived for dimensionality reduction via the eigendecomposition of the matrix P, recent works (van Dijk, D. et al., 2018, Cell 174, 716-729.e27; Lindenbaum, O et al., 2018, In Advances in Neural Information Processing Systems, 1400-1411; Gama, F et al., 2019, In International Conference on Learning Representations (ICLR,). ArXiv:1806.08829; Gao, F et al., 2019, To appear in the Proceedings of the 36th International Conference on Machine Learning, arXiv:1810.03068) have extended the diffusion framework of to allow processing of data features by direct application of the operator P (Coifman, R. R. et al., 2006, Applied and computational harmonic analysis 21, 5-30). These approaches include data denoising and imputation, data generation, and graph embedding with geometric scattering (van Dijk, D. et al., 2018, Cell 174, 716-729.e27; Lindenbaum, O et al., 2018, In Advances in Neural Information Processing Systems, 1400-1411; Gama, F et al., 2019, In International Conference on Learning Representations (ICLR,). ArXiv:1806.08829; Gao, F et al., 2019, To appear in the Proceedings of the 36th International Conference on Machine Learning, arXiv:1810.03068). In these cases, P serves as a smoothing operator, and may be regarded as a generalization of a low-pass filter for either unstructured or graph-structured data. Indeed, consider a vector v∈ [symbol] ^{N} construed as a signal v(xᵢ) over X. Then Pv(xᵢ) replaces the value v(xᵢ) with a weighted average of the values v(xⱼ) for those points xⱼ such that ∥xᵢ−xⱼ∥=O(\sqrt{ε}). Each of these applications, however, uses a time homogeneous matrix P the defines the transition probabilities of a random walk over the dataset X. Computing powers of P runs the walk forward, so that P^{t} gives the transition probabilities of the t-step random walk. Since the same transition probabilities are used for every step of the walk, the resulting diffusion process is time homogeneous. By contrast, a time inhomogeneous diffusion process arises from an inhomogeneous random walk in which the transition probabilities change with every step. Its t-step transition probabilities are given by:

P^{(t)}=PₜPₜ₋₁ . . . P₁,

where P^{(t)} is a time inhomogeneous diffusion operator is composed of many Pₖ is the Markov matrices that encodes the transition probabilities at step k and P^{t} describes transition probabilities for the time homogeneous case (where the resulting matrix is a power of the diffusion operator P). Previous works have applied the time inhomogeneous diffusion operator to data with an explicit time variable, as described in Marshall et al., 2018, Applied and Computational Harmonic Analysis. 45:709-728, attempting to approximates heat diffusion over the time varying manifold M^{d}(τ). The application of time inhomogeneous or homogeneous diffusion operators to learn data topology however, has not been previously explored.

Diffusion Condensation

Diffusion condensation is a dynamic process the builds upon previously established concepts in diffusion filters, diffusion geometry and topological data analysis. The algorithm slowly and iteratively moves points together at a rate determined by the diffusion probabilities between them as described in Brugnone, N. et al. Coarse graining of data via inhomogeneous diffusion condensation, in 2019 IEEE International Conference on Big Data (Big Data), 2624-2633. This iterative process reveals the topology of the underlying geometry and identifies key persistent features that can be directly mapped back onto the underlying dataset. The diffusion condensation approach involves two steps that are iteratively repeated until all points converge:
1. Compute a time inhomogeneous Markov diffusion operator from the data;
2. Apply this operator to the data as a low-pass diffusion filter, moving points towards local centers of gravity.

As established in prior work, the application of the operator P to a vector v averages the values of v over small neighborhoods in the data, as described in van Dijk et al., 2018, Cell, 174:716-729.e27. In the case of data X={x₁, . . . , x_{N}}⊂ [symbol] ⁿ measured from an underlying manifold [symbol] ^{d} with the model xᵢ=ƒ(zᵢ) for zᵢ∈ [symbol] ^{d}, this averaging operator can be directly applied to the coordinate functions ƒ=(ƒ₁, . . . , ƒₙ). Let ƒₖ∈ [symbol] ⁿ be the vector corresponding to the coordinate function ƒₖ evaluated on the data samples, i.e., ƒₖ(zᵢ)=ƒₖ(zᵢ). The resulting description of the data is given by X̅={x̅₁, . . . , x̅_{N}} where x̅ᵢ=(Pf₁(xᵢ), . . . , Pfₙ(xᵢ)). Applications of the operator P to X dampens high frequency variations in the coordinate function and creates smoothed output X̅. The method of the invention considers not only the task of eliminating variability that originates from noise, but also coarse graining the data coordinates to learn the topology of the data. Therefore, the method of the invention gradually eliminates local variability in the data using a time inhomogeneous diffusion process that refines the constructed geometry to coarser resolutions as time progresses. This condensation process proceeds as follows. Let X(0)=X be the original dataset with Markov matrix P₀=P and X(1)=X̅ the coordinate-smoothed data described in the previous paragraph. This process can be iterated to further reduce the variability in the data by computing the Markov matrix P₁ using the coordinate representation X(1). A new coordinate representation X(2) is obtained by applying P₁ to the coordinate functions of X(1). In general, one can apply the process for an arbitrary number of steps, which results in the condensation process. Let X(t) be the coordinate representation of the data after t≥0 steps so that X(t)={xᵢ(t), . . . , x_{N} (t)} with xᵢ(t)=(f₁^{(t)}(zᵢ), . . . , fₙ^{(t)}(zᵢ)), where fₖ⁽⁰⁾=fₖ. X(t+1) is obtained by applying Pₜ, the Markov matrix computed from X(t), to the coordinate vectors fₖ^{(t)}. For t≥0, this process results in

fₖ^{(t+1)}=Pₜfₖ^{(t+1)}=PₜPₜ₋₁ . . . P₁P₀fₖ (Equation 3)

From Equation 3, it can be seen that the coordinate functions of the condensation process at time t+1 are derived from the imposed time inhomogeneous diffusion process P^{(t)}=Pₜ . . . P₀. The low-pass operator Pₜ applies a localized smoothing operation to the coordinate functions fₖ^{(t)}. Over the entire condensation time, however, the original coordinate functions fₖ are smoothed by the cascade of diffusion operators Pₜ . . . P₀. This process adaptively removes the high frequency variations in the original coordinate functions. The effect on the data points X is to draw them towards local barycenters, which are defined by the inhomogeneous diffusion process. Once two or more points collapse into the same barycenter, they are identified as being members of the same topological feature or cluster.

Manifold-Intrinsic Diffusion Condensation Process for Application to Single-Cell RNA-Sequencing Data

In its original form, the diffusion condensation process cannot be applied to scRNAseq data. While useful for general data analysis tasks, this process has limitations: 1) the approach does not work in the non-linear space of the single cell transcriptomic manifold; 2) does not scale to even thousands of data points; 3) does not identify granularities of the topology which meaningfully partition the cellular state space and 4) does not identify pathogenic populations implicated in disease processes. Beyond addressing these limitations, the method of the invention extends the framework to efficiently perform key single cell analysis tasks such as cluster characterization and differential expression analysis.

The method of the invention includes the following significant adaptions for application to single cell data:
1. Dynamically learn the geometry of the single cell manifold with each diffusion filters using spectral entropy;
2. Visualize learned topology via embedding of condensation homology;
3. Use topological activity to identify meaningful granularities for downstream analysis;
4. Implement diffusion operator landmarking, weighted random walks and data merging to efficiently scale to thousands of cells;
5. Implement diffusion condensation with alpha decay kernel for automated cluster characterization and efficient computation of differentially expression genes with condensed transport.

Learning Manifold Geometry Dynamically with Spectral Entropy and t-Step Diffusion Filters

While the initial implementation of diffusion condensation was created to understand multigranular structure of linear data, single cells occupy a highly non-linear space requiring manifold learning strategies (Coifman, R. R. et al., 2006, Applied and computational harmonic analysis 21, 5-30; van Dijk, D. et al., 2018, Cell 174, 716-729.e27; Moon, K. R. et al., 2019, Nature Biotechnology 37, 1482-1492). In single cell data, technical noise, such as drop out and variation, creates measurement artifacts. When building diffusion probabilities on this sort of noisy data, high transition probabilities can be calculated between unrelated cells inappropriately. Thus, directly working with P, fails to acknowledge non-linearities and technical artifacts present within single cell data. Previous work in data diffusion has shown that raising the diffusion probabilities matrix P to the power of t refines these transition probabilities, increasing the chance of transitioning to more related cells (Coifman, R. R. et al., 2006, Applied and computational harmonic analysis 21, 5-30; van Dijk, D. et al., 2018, Cell 174, 716-729.e27; Moon, K. R. et al., 2019, Nature Biotechnology 37, 1482-1492). This powering step allows learning of the relevant non-linear geometry of the data manifold, allowing for spurious neighbors found in the ambient measurement space of cells to be ignored and allowing instead for finding diffusion neighbors that lie on the single cell manifold.

As single cell datasets can often suffer from different types and scales of noise, previous approaches have found that the correct number of t-steps to take must be computed adaptively in a data dependent manner. Previously proposed strategies to select t however, are often slow, as they require trial-and-error approach which rely upon the structure of the underlying dataset. In diffusion condensation, however, the structure of the underlying dataset continuously shifts between granularities due to the repeated application of diffusion filters, making the repeated computation of t necessary and through these techniques computationally unwieldy.

Therefore, in the method of the invention, t is selected adaptively at each condensation iteration using a spectral entropy based approach. Previously, it has been shown that powering the diffusion probabilities P differentially effects the eigenvectors of the powered matrix. While the noisy, high frequency eigenvectors rapidly reduce to zero, the more informative, low frequency eigenvectors diminish much less rapidly, as described in van Dijk et al., 2018, Cell, 174:716-729.e27. This invention was based, in part, on the reasoning that there is a value of t which optimally reduces the noisy information from the high frequency eigenvectors while maintaining the maximum information from the low frequency, informative eigenvectors. To identify this point, the spectral entropy of the diffusion probabilities P was computed when powered to different levels of t. Spectral entropy is defined as the Shannon entropy of normalized eigenvalues, i.e.,

$$S ( P , t ) = - {\sum }_{i} {ψ}_{i}^{t} log ( {ψ}_{i}^{t} ) , \qquad ( Equation 4 )$$

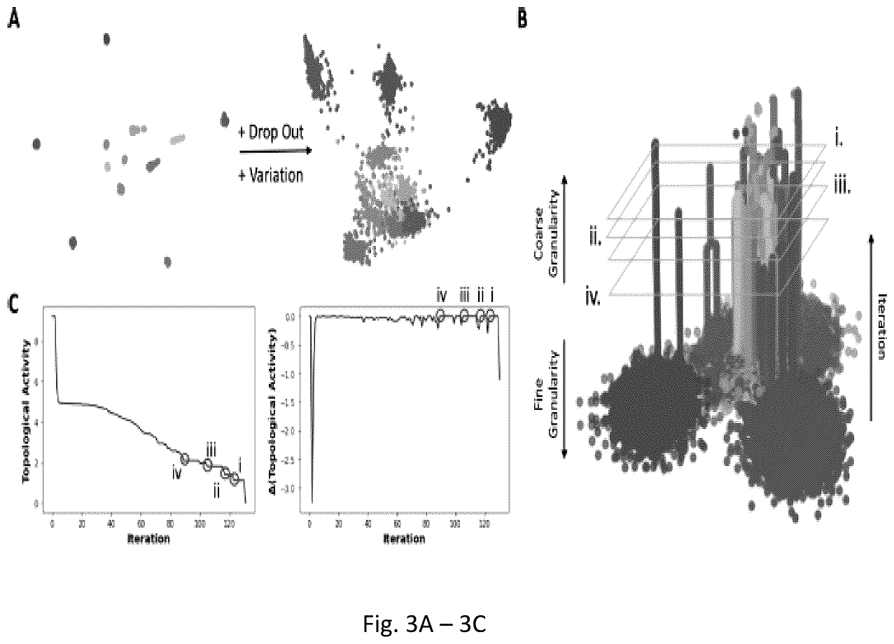

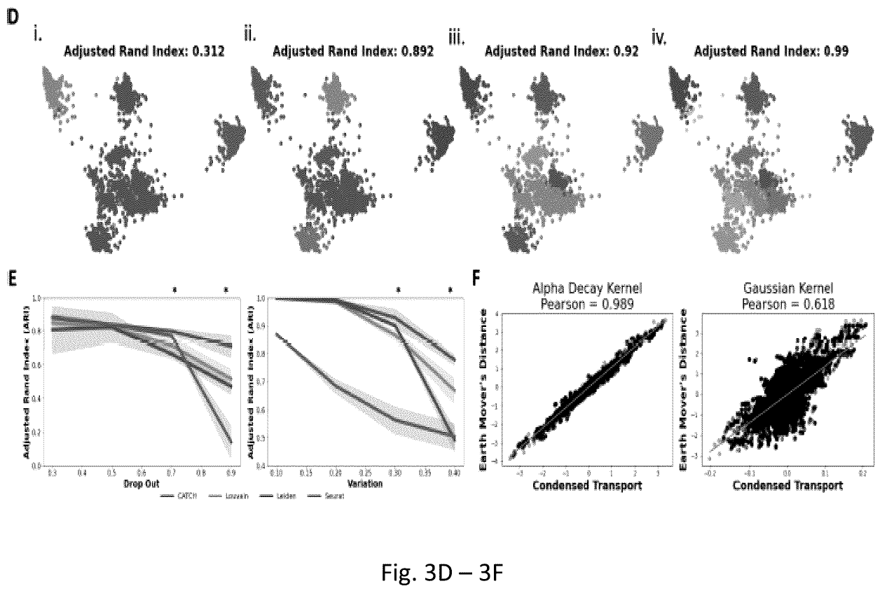

As there is a degree of information loss with each increasing value of t, the point at which this information loss curve stabilizes was identified. While powering to low values of t rapidly decreases spectral entropy as large amount of noise diminish, powering to higher values of t only slowly reduces entropy due to the slower removal of information from informative, low frequency eigenvectors. Taking the point at which this stabilization occurs, optimally allows for adaptive selection of a value of t at each diffusion condensation iteration, allowing the production a diffusion filter which has learned the single cell manifold. In fact, deriving t adaptively in a data driven manner is critical to learning the multigranular cluster structure of data. In order to illustrate this point, synthetic single cell data was generated using splatter as described in Zappia, et al., 2017, Genome Biology, 18. Selecting t via spectral entropy produced a better set of cluster labels than when setting t in a fixed, user-determined manner as shown by the data presented in FIG. 3B. In fact, setting t to 1 does not learn the data manifold or the cluster structure of even fairly noiseless single cell data, revealing the need for selecting a high level of t in an adaptable, data-driven manner. Finally, over successive condensation steps, the complexity of the data decreases and thus requiring lower levels of t to learn, as shown by the data presented in FIG. 3A.

Improving Scalability with Weighted Random Walks, Landmarked Diffusion Operators and Merged Data Points

Repeated computation of a diffusion operator from high dimensional single cell data, powering of this diffusion operator to identify the optimal value of t followed by diffusion filter application via matrix multiplication is computationally expensive. Repeating these computations, potentially hundreds of times, as done by diffusion condensation is unwieldy. In fact, this approach, in its most basic implementation, scales very poorly to high dimensional single cell data with tens of thousands of features and potentially hundreds of thousands of cells. To improve computational efficiency, the following steps are performed:
1. Merge points together that fall below a preset distance threshold ζ to create a cluster, as done in topological data analysis, and weighting random walks to maintain effect of data density;
2. Compute compressed diffusion operator through landmarking as described in Gigante, S. et al. Compressed diffusion. In 2019 13th International conference on Sampling Theory and Applications (SampTA) (IEEE, 2019) to efficiently compute spectral entropy as described in Moon, et al., 2019, Nature Biotechnology, 37:1482-1492.

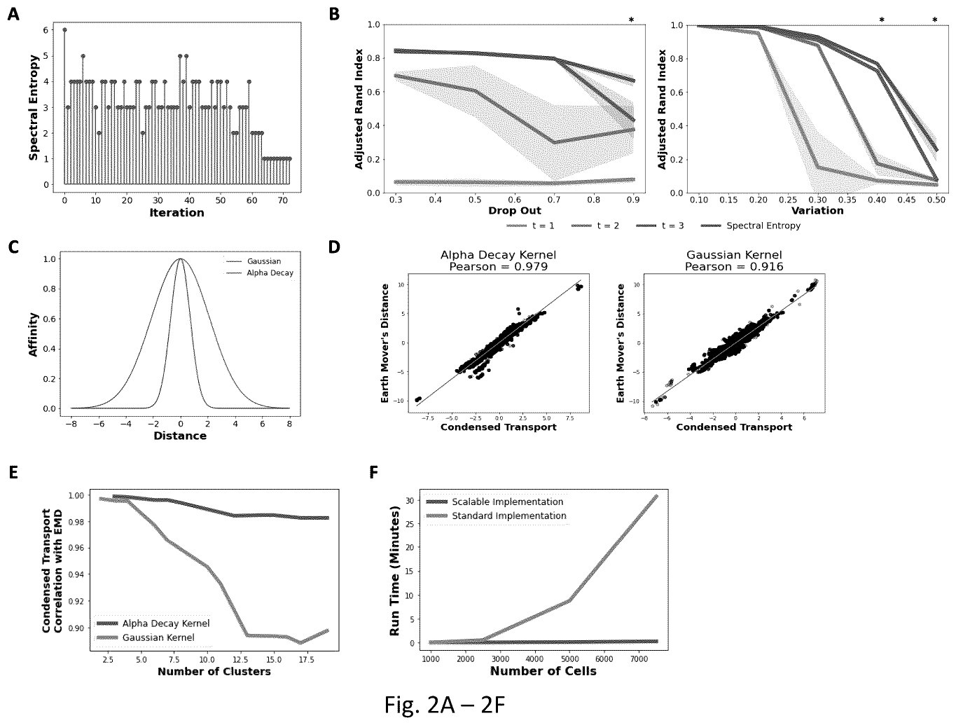

Collectively, these advances drastically improve the computational speed of diffusion condensation (FIG. 2F).

Automated Cluster Characterization in Diffusion Condensation Using α-Decay Kernel

When diffusion condensation merges two cells together at a particular iteration, the newly formed point lies close to the centroid of the original two cells in transcriptomic space. Under specific conditions, the new point is exactly the cluster centroid as delineated in the Proposition below. First, the α-decay kernel is defined as:

$$\begin{matrix}{K}_{a} ( {x}_{i} , {x}_{j} ) = exp \frac{{‖ {x}_{i} - {x}_{j} ‖}^{2}}{{ε}^{α}} , & i , j = 1 , … , N .\end{matrix} \qquad ( Equation 5 )$$

The standard Gaussian kernel function as shown in Equation 2 has an α of 2. The default α-decay kernel meanwhile uses a much higher value (default in this implementation is 40), which converts close distances into affinities much more stringently (FIG. 2C). As α increases to infinity, this kernel function converges almost completely to the box kernel. With this kernel, a set of conditions can be stated under which the diffusion condensation process can be easily characterized.

Proposition 1.

Assume there exists a unique global minimum non-zero distance δᵢ between points xₐ, x_{b} at each iteration i, with the next pair of points at distance at least 6, +τᵢ with 0<τᵢ. Note that xₐ, x_{b} could have multiplicity greater than 1, representing clusters of size >1. Then set the bandwidth to ϵᵢ:=δᵢ+τᵢ/2 at each iteration of the condensation process. For a large enough αα, the diffusion condensation process will maintain two invariants for the first N−1 steps: 1. The number of points will be N−i; 2. Unique points will be located at the centroid of their cluster. Proof. It is easy to verify (1) and (2) hold for step zero. For all i<N and for sufficiently large α, K_{α}(xₖ, xⱼ) becomes arbitrarily close to 1 for (k,j)∈{(a, a), (a, b), (b, a), (b, b)} and 0 otherwise. Exactly one merge occurs at each timestep between points at xₐ and x_{b}. Given Pi as described above, they merge to the point

$$\frac{\vert {x}_{a} \vert {x}_{a} + \vert {x}_{b} \vert {x}_{b}}{\vert {x}_{a} \vert + \vert {x}_{b} \vert} ,$$

i.e. the cluster centroid. By induction (1) and (2) hold for all i<N.

In this setting, the condensation process always converges in exactly N−1 steps. In practice, shorter convergence times are desired as there are many fewer than N−1 interesting levels of clustering. For example, for 50,498 cells, a set of parameters was found that allowed for convergence in 150 steps. For this reason a larger bandwidth 6 is used which leads to much faster convergence and gives cluster centers at each level that are close to but not exactly the cluster centroids of the points they represent. Another factor is the setting of the α parameter. The data in FIG. 3C empirically demonstrates that α-decay kernel implementation better approximates mean expression levels of clusters than the Gaussian kernel implementation.

Efficient Differential Expression Analysis Via Approximation of Wasserstein Earth Mover's Distance

Identifying signatures of pathogenic populations via differential expression analysis is a key component of identifying the mechanisms of disease. Previously, Wasserstein EMD has been used to identify genes that are differentially expressed, as described in van Dijk et al., 2018, Cell, 174:716-729.e27. While most EMD methods to compute differentially expressed genes transport all genes across the expression landscape to compute true costs, the method of the invention is based in part on the reasoning that each iteration of the diffusion condensation process produces a summarization of the original feature space that can be used to approximate the ground truth EMD in a more scalable manner. Specifically, cluster characterization was used to optimal transport on a tree metric.

EMD, or 1-D Wasserstein distance, is a measure of distance between two distributions. For a given ground distance, the Wasserstein distance between distributions can be thought of as the minimal total distance needed to move one distribution to the other. Let μ, ν be two distributions on a measurable space Ω with metric d(·,·), and Π(μ, ν) be the set of joint distributions π on the space D. Ω×Ω, such that for any subset ω⊂Ω, π(ω×Ω)=μ(ω) and π(Ω×ω)=ν(ω). The 1-Wasserstein distance W_{d} also known as the earth mover's distance (EMD) is defined as:

$${W}_{d} ( μ , v ) := {inf}_{π ∈ \prod ( μ , v )} {\int }_{Ω \times Ω} d ( x , y ) π ( dx , dy ) . \qquad ( Equation 6 )$$

When μ, ν are discrete distributions over points in [symbol] ^{d}, of size m, n respectively, this can be equivalently expressed in matrix notation as:

$$\begin{matrix}{W}_{d} ( μ , v ) := {min}_{\prod \geq 0} {\sum }_{i = 1}^{m} {\sum }_{j = 1}^{n} {\prod }_{ij} d ( {x}_{i} , {x}_{j} ) \\ \begin{matrix}subject to : {\sum }_{i = 1}^{m} {\prod }_{ij} = {v}_{j} , & ∀ j ∈ \{ 1 , … , n \}\end{matrix} \\ \begin{matrix}{\sum }_{j = 1}^{n} {\prod }_{ij} = {μ}_{j} , & ∀ j ∈ \{ 1 , … , n \}\end{matrix}\end{matrix} \qquad ( Equation 7 )$$

For general ground distances this is computable using the Hungarian algorithm in Õ(n³) time. Intuitively, the difficulty in computing the optimal transport is finding the map H which optimizes the cost within the constraints. However, for a tree metric, this optimal map is easy to compute in closed form because there is only a single path (through the tree) between pairs of points. This single path between pairs of points results in a reduced computational complexity of Õ(n). This is best understood using the Kantorovich-Rubenstein dual form of the Wasserstein distance:

$${W}_{d} ( μ , v ) = {sup}_{f : {‖ f ‖}_{L} \leq 1} {\int }_{Ω} f ( x ) d μ - {\int }_{Ω} f ( x ) d v \qquad ( Equation 8 )$$

where the witness function ƒ: Ω→ [symbol] and ∥·∥_{L} denotes the Lipschitz norm. This dual form holds under a few minor conditions which hold for the spaces considered here. Given some rooted tree T with strictly non-negative edge lengths, the natural tree metric d_{T}(x,y) is defined as the length of the unique path between nodes x, y. The mass of a distribution on a subtree Tᵣ rooted at node r is denoted as μ(Tᵣ)=Σ_{x∈T}_{ᵣ}μ(x). For each node ν∈T its associated parent edge is denoted as e_{ν} with weight w_{ν}. In this setting, it is easy to construct the optimal witness function in Equation 8. Without loss of generality, one starts at the root r and builds ƒ such that ƒ(r)=0 and for each edge e(u, ν) where u is a parent of ν, ƒ(ν)=ƒ(u)+wₑ sign(μ(T_{ν}) ν(T_{ν})). Given this construction, it is easy to see that the Wasserstein distance with tree ground distance has the following closed form:

$${W}_{{d}_{T}} ( μ , v ) = {\sum }_{υ ∈ T} {w}_{υ} \vert μ ( {T}_{υ} ) - v ( {T}_{υ} ) \vert . \qquad ( Equation 9 )$$

The question then comes to: what are useful tree metrics? An ideal tree metric that has low distortion of Euclidean space and is scalable to high dimensions. QuadTree is a tree metric algorithm designed to approximate the optimal transport distance between discrete measures with Euclidean ground distance by recursively partitioning space into hypercubes, but does not scale well with dimension (Indyk, P. et al., 2003, 3rd International Workshop on Statistical and Computational Theories of Vision). Specifically, assume, without loss of generality, that the data lies in the [0, 1]^{d} hypercube, then at each level h∈[0, H) divide the space into 2^{d}^{^{h}} hypercubes with side length 2^{-h}. This forms an H-level tree with each node representing a hypercube. If the center of the hypercube is randomly shifted, then the QuadTree distance W_{d}_{_{QT}} has distortion at most O(d log 1/τ) where τ is the minimum distance between datapoints, i.e.

c·(d log τ)W_{d}_{_{QT}}(μ,ν)≤W_{∥·∥}_{₂}(μ,ν)≤C·(d log τ)W_{d}_{_{QT}}(μ,ν) (Equation 10)

some constants c, C in expectation (Indyk, P. et al., 2003, 3rd International Workshop on Statistical and Computational Theories of Vision).

However, QuadTree distance scales poorly as it is computed in O(Nd·log(d1/τ)). In the high dimensional setting, such as snRNAseq data, the poor scaling with respect to d both computationally and in the approximation is undesirable. In this setting (Le, T et al., 2019, In Advances in neural information processing systems, 12304-12315) suggests sampling trees using furthest point clustering (Gonzalez, T. F. 1985, Theoretical Computer Science 38, 293-306). Furthermore, (Backurs, A. et al., 2020, Scalable nearest neighbor search for optimal transport, 1910.04126) implements FlowTree, a small modification to QuadTree that makes tree Wasserstein distances significantly more accurate with the addition of small additional computational cost. CATCH implements a new formulation of EMD over the diffusion condensation tree. For two diffusion condensation clusters a, b located at Cₐ, C_{b} respectively the condensed transport distance between them is defined as:

$${W}_{CT} ( a , b , T ) = {‖ {C}_{a} - {C}_{b} ‖}_{2} + {\sum }_{e ( υ , υ ) ∈ {T}_{a}} {w}_{e} \cdot a ( {T}_{υ} ) + {\sum }_{e ( υ , υ ) ∈ {T}_{b}} {w}_{e} \cdot b ( {T}_{υ} ) \qquad ( Equation 11 )$$

where wₑ:=2^{-h}∥C_{ν}−Cᵤ∥₂ for edge e(u, ν) at depth h and a(x), b(x) are defined as indicator functions of their respective clusters. This leads to the following proposition stating that no matter how close one is to the settings in Proposition 1, W_{CT} still represents a valid tree Wasserstein distance between clusters.

Proposition 2.

The condensed transport distance W_{CT}, for any diffusion condensation tree T, defines a valid Wasserstein distance over a tree ground distance for any two clusters in that tree. Proof. This is shown by constructing the associated tree metric d_{CT} on an arbitrary condensation tree T_{CT} and conclude by showing that

$${W}_{{d}_{{T}_{C T}}}$$

is equivalent to W_{CT}. Begin by rooting the tree at a node representing Cₐ with two children, the root of Tₐ named rₐ and C_{b}. The edge e(Cₐ, rₐ) has weight 0 and the edge (Cₐ, C_{b}) has weight ∥Cₐ−C_{b}∥₂. The node C_{b} will have a single child node the root of Tₐ named r_{b}, and is connected by an edge of length zero. All other nodes will be defined as in Tₐ and T_{b} with associated edge weights. It is easy to verify that the path measure over T_{CT} construction represents a valid distance d_{CT}. Finally, the Wasserstein distance was verified with a ground distance of d_{CT} is equivalent to W_{CT} as defined in Equation 11. Indeed, because a skip connection was added in the tree to directly connect nodes a, b with an edge of length ∥Cₐ−C_{b}∥₂ and since a(T_{ν}) for ν∈T_{b} is always zero and vice versa, the following is obtained

$$\begin{matrix}{W}_{{a}_{CT}} ( a , b ) = {\sum }_{e ( υ , υ ) ∈ {T}_{CT}} {w}_{e} \vert a ( {T}_{υ} ) - b ( {T}_{υ} ) \vert \\ = {w}_{e ( {C}_{a} , {C}_{b} )} \vert a ( {T}_{{C}_{b}} ) - b ( {T}_{{C}_{b}} ) \vert + {\sum }_{e ( υ , υ ) ∈ {T}_{a}} {w}_{e} \vert a ( {T}_{υ} ) - b ( {T}_{υ} ) \vert + {\sum }_{e ( υ , υ ) ∈ {T}_{b}} {w}_{e} \vert a ( {T}_{υ} ) - b ( {T}_{υ} ) \vert \\ = {‖ {C}_{a} - {C}_{b} ‖}_{2} \vert 0 - 1 \vert + {\sum }_{e ( υ , υ ) ∈ {T}_{a}} {w}_{e} \vert a ( {T}_{υ} ) - 0 \vert + {\sum }_{e ( υ , υ ) ∈ {T}_{b}} {w}_{e} \vert 0 - b ( {T}_{υ} ) \vert \\ = {‖ {C}_{a} - {C}_{b} ‖}_{2} + {\sum }_{e ( υ , υ ) ∈ {T}_{a}} {w}_{e} \cdot a ( {T}_{υ} ) + {\sum }_{e ( υ , υ ) ∈ {T}_{b}} {w}_{e} \cdot b ( {T}_{υ} ) \\ = {W}_{CT} ( a , b , T ) .\end{matrix} \qquad ( Equation 12 )$$

Note that W_{CT} does not calculate the Wasserstein distance over the same tree for each set of clusters, and as shown in (Backurs, A. et al., 2020, Scalable nearest neighbor search for optimal transport, 1910.04126) this often improves the accuracy as compared. In addition, it is useful conceptually but not essential that the cluster centers Cₐ, C_{b} are near the cluster centroids. Proposition 1 delineated the setting where this holds exactly, but these parameters are impractical for efficient computation requiring n−1 diffusion steps. Instead, the system has been developed with centers that are close to the centroids but are efficiently computable in many fewer diffusion steps. This formulation is similar to the standard Wasserstein distance with tree ground distance as in Equation 9, but simplified and optimized for the case of comparing clusters which are elements of the tree metric. The algorithm contains two changes. First, a skip connection was added in the tree to directly connect nodes a, b with an edge of length ∥Cₐ−C_{b}∥₂. Next, a(T_{ν}) for ν∈T_{b} is always zero and vice versa, thus simplifying the second and third terms. These two optimizations give an algorithm that is efficient in high dimensions and is effective empirically and across granularities as demonstrated in the data provided in FIGS. 2 and 3.

Combining Diffusion Condensation with MELD to Identify Populations Enriched in Disease Phases

Identifying and characterizing rare pathogenic cell populations has been a persistent computational problem in single cell analysis, making it difficult to create cell type specific signatures of disease. To address this concern, CATCH was applied in conjunction with MELD (Burkhardt, et al. 2020, bioRxiv), a tool that identifies regions of the manifold enriched for cells from a specific condition. This combined approach identified cell states enriched in disease, rapidly characterized pathogenic expression signatures and finally predicted cellular interactions between pathogenic populations, uncovering potential therapeutic targets for intervention.

While diffusion condensation and topological activity analysis may be adept at identifying populations of cells across granularities, identifying granularities that isolate disease enriched populations remains a problem. In order to identify groups of cells enriched in specific disease states, the disclosed topological approach may be combined with MELD (Burkhardt, et al. 2020, bioRxiv) MELD creates a joint graph of the samples being compared, and returns a relative likelihood that quantifies the probability that each cell state in the graph is more likely in a particular disease condition. This likelihood score is found by first computing a cell-cell graph before creating an indicator signal for each of the two conditions. In some embodiments, the disease phase of origin may be used as the indicator signal, labeling the cells not from that phase as a control. Then MELD may be used to smooth, or low-pass filter these signals over the cell-cell graph using the heat kernel to calculate the relative likelihood of each condition over the cellular manifold. The density estimates of both conditions are then inverted via Bayes formula to create a relative likelihood of the condition given the cell. This likelihood score highlights regions of the manifold enriched in different conditions. Using the MELD likelihood scores, it is possible to identify cellular subsets enriched in particular disease phases and furthermore, identify topological features that maximally separate disease-phase signals.

Systems

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

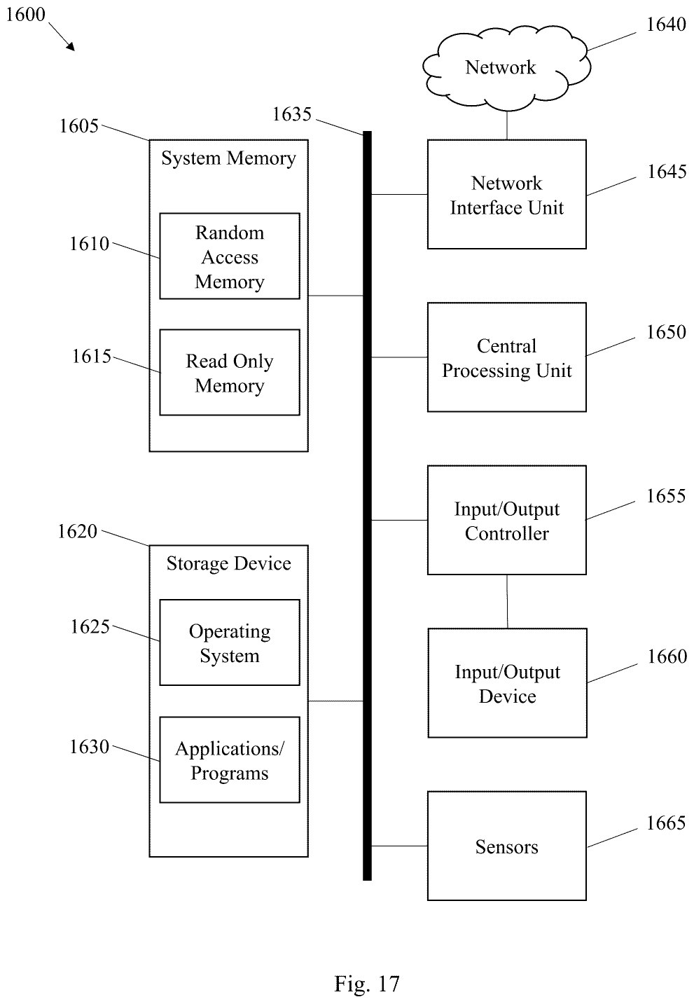

FIG. 17 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 17 depicts an illustrative computer architecture for a computer 1600 for practicing the various embodiments of the invention. The computer architecture shown in FIG. 17 illustrates a conventional personal computer, including a central processing unit 1650 (“CPU”), a system memory 1605, including a random access memory 1610 (“RAM”) and a read-only memory (“ROM”) 1615, and a system bus 1635 that couples the system memory 1605 to the CPU 1650. A basic input/output system containing the basic routines that help to transfer information between elements within the computer, such as during startup, is stored in the ROM 1615. The computer 1600 further includes a storage device 1620 for storing an operating system 1625, application/program 1630, and data.

The storage device 1620 is connected to the CPU 1650 through a storage controller (not shown) connected to the bus 1635. The storage device 1620 and its associated computer-readable media provide non-volatile storage for the computer 1600. Although the description of computer-readable media contained herein refers to a storage device, such as a hard disk or CD-ROM drive, it should be appreciated by those skilled in the art that computer-readable media can be any available media that can be accessed by the computer 1600.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1600 may operate in a networked environment using logical connections to remote computers through a network 1640, such as TCP/IP network such as the Internet or an intranet. The computer 1600 may connect to the network 1640 through a network interface unit 1645 connected to the bus 1635. It should be appreciated that the network interface unit 1645 may also be utilized to connect to other types of networks and remote computer systems.

The computer 1600 may also include an input/output controller 1655 for receiving and processing input from a number of input/output devices 1660, including a keyboard, a mouse, a touchscreen, a camera, a microphone, a controller, a joystick, or other type of input device. Similarly, the input/output controller 1655 may provide output to a display screen, a printer, a speaker, or other type of output device. The computer 1600 can connect to the input/output device 1660 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 1620 and/or RAM 1610 of the computer 1600, including an operating system 1625 suitable for controlling the operation of a networked computer. The storage device 1620 and RAM 1610 may also store one or more applications/programs 1630. In particular, the storage device 1620 and RAM 1610 may store an application/program 1630 for providing a variety of functionalities to a user. For instance, the application/program 1630 may comprise many types of programs such as a word processing application, a spreadsheet application, a desktop publishing application, a database application, a gaming application, internet browsing application, electronic mail application, messaging application, and the like. According to an embodiment of the present invention, the application/program 1630 comprises a multiple functionality software application for providing word processing functionality, slide presentation functionality, spreadsheet functionality, database functionality and the like.

The computer 1600 in some embodiments can include a variety of sensors 1665 for monitoring the environment surrounding and the environment internal to the computer 1600. These sensors 1665 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

Algorithm

In some embodiments, the method comprises using a CATCH algorithm as described in detail above to determine if the level of one or more cell population or biomarker in a biological sample. In some embodiments, the level of one or more cell population or biomarker in a biological sample is determined to be statistically different than the level of the one or more cell population or biomarker in a control sample. In some embodiments, the CATCH algorithm is a trained algorithm. In various embodiments, the CATCH algorithm includes one or more: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms. Such algorithms may be used in supervised or unsupervised learning modes. In various embodiments, the CATCH algorithm according to the disclosure can be used to determine the extent, severity, or stage of disease, to determine the right treatment approach (e.g., disease-specific therapy, surgical intervention), to select the appropriate dose for a medical treatment, to determine whether a patient is likely to respond to a particular medical or surgical treatment, to monitor response to treatment, or to monitor disease progression.

In some embodiments, the methods according to the disclosure include deriving a numerical value, index or score from the CATCH algorithm or mathematical formula. In some embodiments, the derived numerical value can serve as a cut off value for distinguishing between two or more potential outcomes (e.g., high or low risk of disease presence, progression or recurrence or stage of disease.) In some embodiments, a derived numerical value serves as a cutoff value for a level of at least one cell population or biomarker. In some embodiments, a derived numerical value serves as a cutoff value for a level of at least one cell population or biomarker in order determine the extent, severity, or stage of disease. In some embodiments, a derived numerical value serves as a cutoff value for a level of at least one cell population or biomarker in order to determine the right treatment approach (e.g., disease-specific therapy, surgical intervention). In some embodiments, a derived numerical value serves as a cutoff value for one cell population or biomarker in order to select the appropriate dose for a medical treatment.) In some embodiments, a derived numerical value serves as a cutoff value for a level of at least one cell population or biomarker in order to determine whether a patient is likely to respond to a particular medical or surgical treatment. In some embodiments, a derived numerical value serves as a cutoff value for a level of at least one cell population or biomarker in order to monitor response to treatment. In some embodiments, a derived numerical value serves as a cutoff value for a level of at least one cell population or biomarker in order to monitor disease progression.

In some embodiments, the method of the invention involves a step of comparing the level of at least one of cell population or biomarker identified or quantified in a biological sample obtained from a subject to a predetermined cut off or to the level of at least one cell population or biomarker in a comparator control (i.e., positive control, negative control, historical norm, baseline level or reference value). In some embodiments, an increase in the level of at least one cell population or biomarker in a biological sample from the subject under study relative to the predetermined cut-off or the level of at least one of cell population or biomarker in a comparator control sample is indicative of a disease or disorder associated with the presence of the cell population or biomarker, i.e., it is an indication that said subject is suffering from a disease or disorder associated with the presence of the cell population or biomarker or has a predisposition to develop a disease or disorder associated with the presence of the cell population or biomarker. In some embodiments, a decrease in the level of at least one cell population or biomarker in a biological sample from the subject under study relative to the predetermined cut-off or the level of the at least one of cell population or biomarker in a comparator control sample is indicative of a disease or disorder associated with the absence of the cell population or biomarker, i.e., it is an indication that said subject is suffering from a disease or disorder associated with the absence of the cell population or biomarker or has a predisposition to develop a disease or disorder associated with the absence of the cell population or biomarker.

### Diagnostic Index

In one embodiment, the present invention relates to the identification of one or more cell population or biomarker profile and optionally one or more additional clinical features to generate diagnostic indexes for diagnosing a disease or disorder or risk of a disease or disorder. Accordingly, the present invention features methods for identifying subjects who have or are at risk of developing a disease or disorder by detection of at least one cell population or biomarker and assessing the clinical factors disclosed herein. These factors, or otherwise health profile, are also useful for monitoring subjects undergoing treatments and therapies, and for selecting or modifying therapies and treatments to alternatives that would be efficacious in subjects determined by the methods of the invention to have a disease or disorder associated with the presence or absence of a rare cell population or an increased risk of developing a disease or disorder associated with the presence or absence of a rare cell population.

The present invention provides an index of for use in patient monitoring or diagnostics. In some embodiments, the index is calculated as a function of multiple markers, biomarkers or factors that strongly correlate to a specific disease or disorder associated with the presence or absence of a rare cell population. These factors may include a combination of clinical factors and relative cell population levels.

The risk of developing a specific disease or disorder associated with the presence or absence of a rare cell population can be assessed by measuring one or more of the factors described herein, and comparing the presence and values of the factors to reference or index values. Such a comparison can be undertaken with mathematical algorithms or formula in order to combine information from results of multiple individual factors and other parameters into a single measurement or diagnostic index. Subjects identified as having a specific disease or disorder associated with the presence or absence of a rare cell population or an increased risk of a specific disease or disorder associated with the presence or absence of a rare cell population can optionally be selected to receive counseling, an increased frequency of monitoring, or treatment regimens, or administration of alternative therapeutic compounds. For example, in one embodiment, a subject identified as having high microglia-derived IL-1β and high pro-angiogenic astrocytes may be administered a treatment for late-stage neovascular AMD (e.g., an anti-VEGF injection or an IL-1β inhibitor), whereas a subject identified as having a microglial subset and astrocyte subset, enriched in the early phase of dry AMD may receive an increased frequency of monitoring for disease progression.

The factors of the present invention can thus be used to generate a health profile or signature of subjects: (i) who do not have and are not expected to develop a specific disease or disorder associated with the presence or absence of a rare cell population and/or (ii) who have or expected to develop a specific disease or disorder associated with the presence or absence of a rare cell population. The health profile of a subject can be compared to a predetermined or reference profile to diagnose or identify subjects at risk for developing a specific disease or disorder associated with the presence or absence of a rare cell population, to monitor the response to a therapeutic treatment (e.g. an antibiotic or a chemotherapeutic agent), and to monitor the effectiveness of a treatment or preventative measure for a specific disease or disorder associated with the presence or absence of a rare cell population. Data concerning the factors of the present invention can also be combined or correlated with other data or test results, such as, without limitation, measurements of clinical parameters or other algorithms for a specific disease or disorder associated with the presence or absence of a rare cell population.

In one embodiment the diagnostic index for diagnosing a specific disease or disorder associated with the presence or absence of a rare cell population is provided which integrates results from two or more tests for diagnosing a specific disease or disorder associated with the presence or absence of a rare cell population thereby providing a scoring system to be used in distinguishing subjects having or at risk for a specific disease or disorder associated with the presence or absence of a rare cell population. Examples of the diagnostic tests that may be integrated to generate the diagnostic index include, but are not limited to, detecting the level of one or more additional biomarker for a specific disease or disorder associated with the presence or absence of a rare cell population. In one embodiment, at least two diagnostic tests are used in generating the index. The two or more diagnostic tests used in generating the index can diagnose a specific disease or disorder associated with the presence or absence of a rare cell population based on identification of changes in the same or different directions in a test sample relative to a comparator control. For example, in one embodiment, two or more diagnostic tests both assess an increase in the detected marker as compared to a comparator control. In another embodiment, at least one diagnostic test detects an increase in the detected marker as compared to a comparator control and at least one diagnostic test detects a decrease in the detected marker as compared to a comparator control.

In one embodiment, the diagnostic index includes at least one additional factor. Exemplary additional factors that can be included in the diagnostic index include, but are not limited to, age, sex, race, family history and previous history of a specific disease or disorder associated with the presence or absence of a rare cell population. Information obtained from the methods of the invention described herein can be used alone, or in combination with other information (e.g., age, race, sexual orientation, vital signs, blood chemistry, etc.) from the subject or from a biological sample obtained from the subject.

One of skill in the art recognizes that for an individual test statistical analysis can be performed on a reference or normative population sample of cells to determine confidence levels of having a specific disease or disorder associated with the presence or absence of a rare cell population based on the results of that test. Accordingly for each test, a scale can be arbitrarily partitioned into regions having scores such that a correct combination of the scores provides a diagnostic index having a certain degree of confidence. The partitioning can be performed by conventional classification methodology including, but not limited to, histogram analysis, multivariable regression or other typical analysis or classification techniques. For example, one skilled in the art recognizes that multi-variable regression analysis may be performed to generate this partitioning or to analyze empirical/arbitrary partitioning in order to determine whether the composite clinical index has a higher degree of significance than each of the individual indices from respective tests.

Various embodiments of the present invention describe mechanisms configured to monitor, track, and report levels of at least one clinical factor and optionally one or more biomarkers of a specific disease or disorder associated with the presence or absence of a rare cell population for use in generating a diagnostic index of an individual at multiple time points. In one embodiment, the system allows for the collection of data from multiple samples from an individual. The system can notify the user/evaluator about the likelihood of risk of developing a specific disease or disorder associated with the presence or absence of a rare cell population when a change (i.e. increase or decrease) in the diagnostic is detected in subsequent samples from a single individual. For example, in some implementations, the system records the diagnostic index entered into the system by the user/evaluator or automatically recorded by the system at various timepoints during a treatment regimen and applies algorithms to recognize patterns that predict whether the individual is at high risk of developing a specific disease or disorder associated with the presence or absence of a rare cell population in the absence of intervening treatment. The algorithmic analysis, for example, may be conducted in a central (e.g., cloud-based) system. Data uploaded to the cloud can be archived and collected, such that learning algorithms refine analysis based upon the collective data set of all patients. In some implementations, the system combines quantified clinical features and physiology to aid in diagnosing risk objectively, early, and at least semi-automatically based upon collected data.

Methods

In one embodiment, the systems and methods disclosed herein may be used in biomarker identification, for example for identifying tumor cells from a sample to diagnose cancer or metastasis or for identifying biomarkers to determine additional information about the prognosis or stage of a diagnosed cancer. The systems and methods described herein may be particularly useful for characterizing rare cell populations, including, but not limited to rare pathogenic cell populations.

In one embodiment, the sample is a biological sample or a patient sample.

In one embodiment, the assay is for use in diagnosing a disease or disorder in the subject, monitoring the progression of a disease or disorder in the subject providing a disease prognosis, or evaluating the effects of a treatment provided to a subject.

Determining Effectiveness of Therapy or Prognosis

In one aspect, the CATCH can be incorporated into an assay used to monitor the effectiveness of treatment or the prognosis of disease (e.g., a disease or disorder associated with a rare cell population or a biomarker). In some embodiments, the level of one or more rare cell population in a test sample obtained from a treated patient can be compared to the level from a reference sample obtained from that patient before initiation of a treatment. Clinical monitoring of treatment typically entails that each patient serves as his or her own baseline control. In some embodiments, test samples are obtained at multiple time points following administration of the treatment. In these embodiments, measurement of level of one or more rare cell population in the test samples provides an indication of the extent and duration of in vivo effect of the treatment.

Measurement of cell population levels allow for the course of treatment of a disease to be monitored. The effectiveness of a treatment regimen for a disease can be monitored by detecting one or more cell population or biomarker in an effective amount from samples obtained from a subject over time and comparing the level or amount of the cell population or biomarker detected. For example, a first sample can be obtained before the subject receives treatment and one or more subsequent samples are taken after or during treatment of the subject. Changes in cell population levels or biomarker levels across the samples may provide an indication as to the effectiveness of the therapy.

In some embodiments, the disclosure provides a method for monitoring cell population or biomarker levels in response to treatment. For example, in certain embodiments, the disclosure provides for a method of determining the efficacy of treatment in a subject, by measuring the levels of one or more cell population or biomarker as described herein. In some embodiments, the level of the one or more cell population or biomarker can be measured over time, where the level at one timepoint after the initiation of treatment is compared to the level at another timepoint after the initiation of treatment. In some embodiments, the level of the one or more cell population or biomarker can be measured over time, where the level at one timepoint after the initiation of treatment is compared to the level before initiation of treatment.

In some embodiments, CATCH can be used to identify therapeutics or drugs that are appropriate for a specific subject. For example, a test sample from the subject can be exposed to a therapeutic agent or a drug, and the level of one or more cell population or biomarker can be determined. Levels of the one or more cell population or biomarker can be compared to a sample derived from the subject before and after treatment or exposure to a therapeutic agent or a drug or can be compared to samples derived from one or more subjects who have shown improvements relative to a disease as a result of such treatment or exposure. Thus, in one aspect, the disclosure provides a method of assessing the efficacy of a therapy with respect to a subject comprising taking a first measurement of a cell population, a biomarker or a biomarker panel in a first sample from the subject; effecting the therapy with respect to the subject; taking a second measurement of the cell population, biomarker or a biomarker panel in a second sample from the subject and comparing the first and second measurements to assess the efficacy of the therapy.

Accordingly, treatments or therapeutic regimens for use in the treatment of a disease or disorder (e.g, a disease or disorder associated with a pathogen, or cancer) can be selected based on the amounts of a specific cell population, biomarker or a biomarker panel in one or more sample obtained from the subjects and compared to a reference value. Two or more treatments or therapeutic regimens can be evaluated in parallel to determine which treatment or therapeutic regimen would be the most efficacious for use in a subject to treat, delay onset, or slow progression of a disease. In various embodiments, a recommendation is made on whether to initiate or continue treatment of a disease.

A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example, prognosis can be expressed as a percent chance that a patient will survive after one year, five years, ten years or the like. Alternatively, prognosis may be expressed as the number of years, on average that a patient can expect to survive as a result of a condition or disease. The prognosis of a patient may be considered as an expression of relativism, with many factors affecting the ultimate outcome. For example, for patients with certain conditions, prognosis can be appropriately expressed as the likelihood that a condition may be treatable or curable, or the likelihood that a disease will go into remission, whereas for patients with more severe conditions, prognosis may be more appropriately expressed as likelihood of survival for a specified period of time. Additionally, a change in a clinical factor from a baseline level may impact a patient's prognosis, and the degree of change in level of the clinical factor may be related to the severity of adverse events. Statistical significance is often determined by comparing two or more populations and determining a confidence interval and/or a p value.

Multiple determinations of cell population or biomarker level can be made, and a temporal change in cell population or biomarker level can be used to determine a prognosis. For example, comparative measurements are made of the cell population or biomarker level in a patient at multiple time points, and a comparison of the cell population or biomarker level at two or more time points may be indicative of a particular prognosis.

In certain embodiments, the cell population or biomarker level is used as indicators of an unfavorable prognosis. According to the current disclosure, the determination of prognosis can be performed by comparing the measured cell population or biomarker level to levels determined in comparable samples from healthy individuals or to levels corresponding with favorable or unfavorable outcomes. The cell population or biomarker level obtained may depend on a number of factors, including, but not limited to, the type of body fluid sample used and the type of disease a patient is afflicted with. According to the method, values can be collected from a series of patients with a particular disorder to determine appropriate reference ranges of cell population or biomarker levels for that disorder.

In some embodiments the level of one or more cell population or biomarker in a test sample from a patient relates to the prognosis of a patient in a continuous fashion, the determination of prognosis can be performed using statistical analyses to relate the determined cell population or biomarker level to the prognosis of the patient. A skilled artisan is capable of designing appropriate statistical methods. For example, the methods may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses. The approach by Giles et. al., British Journal of Hemotology, 121:578-585, is exemplary. As in Giles et al., associations between categorical variables (e.g., miRNA levels and clinical characteristics) can be assessed via cross-tabulation and Fisher's exact test. Unadjusted survival probabilities can be estimated using the method of Kaplan and Meier. The Cox proportional hazards regression model also can be used to assess the ability of patient characteristics (such as cell population levels) to predict survival, with ‘goodness of fit’ assessed by the Grambsch-Therneau test, Schoenfeld residual plots, martingale residual plots and likelihood ratio statistics. These statistical analyses can be performed using a statistical program. Estimates can then be used to obtain probabilities of surviving from one to 24 months given the patient's covariates. The program can make use of estimated probabilities to create a graphical representation of a given patient's predicted survival curve. In certain embodiments, the program also provides 6-month, 1-year and 18-month survival probabilities. A graphical interface can be used to input patient characteristics in a user-friendly manner. In some embodiments of the disclosure, multiple prognostic factors, including cell population levels, are considered when determining the prognosis of a patient. For example, the prognosis of a subject with age-related macular degeneration (AMD) may be determined based on the level a microglia inflammasome-related signature. For example, in some embodiments, a microglia inflammasome-related signature is indicative of a poorer prognosis for a subject having AMD whereas the presence or levels of a microglial subset and astrocyte subset may be indicative of a better prognosis for a subject having AMD.

In certain embodiments, other prognostic factors may be combined with the level of one or more cell population or other biomarkers in the algorithm to determine prognosis with greater accuracy. Exemplary additional prognostic factors may include one or more prognostic factors selected from the group consisting of cytogenetics, performance status, age, gender, and contemporary diagnosis.

Treatments

In some embodiments, the methods of the invention are used to identify a cell population or biomarker in a sample. In some embodiments, the cell population or biomarker is associated with a disease or disorder. For example, in some embodiments the cell population is associated with a rare pathogen. In some embodiments the cell population is associated with cancer. In some embodiments the cell population is associated with an autoimmune or inflammatory disease or disorder.

In one aspect, the disclosure provides a method of diagnosing, treating or preventing a disease or disorder associated with an altered level of a cell population. In some embodiments, the method comprises administering to the subject an effective amount of a pharmaceutical agent for the treatment of a disease or disorder identified as associated with an altered level of a specific cell population, including, but not limited to, diseases or disorders associated with the inflammatory process, pathogens, and cancers.

Exemplary inflammatory diseases and disorder that can be diagnosed, treated or monitored for treatment include, but are not limited to, autoimmune diseases, inflammatory diseases, diseases and disorders associated with pathogens and cancer.

Exemplary autoimmune and inflammatory diseases include, but are not limited to, age-related macular degeneration, arthritis (e.g., rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, psoriasis vulgaris, inverse psoriasis, erythrodermic psoriasis, seborrheic psoriasis and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis, respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions, allergic reaction, eczema including allergic or atopic eczema, asthma such as asthma bronchiale, bronchial asthma, and auto-immune asthma, conditions involving infiltration of T cells and chronic inflammatory responses, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP) and autoimmune or immune-mediated thrombocytopenia such as idiopathic thrombocytopenic purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine ophthalmopathy, uveoretinitis, chorioretinitis, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia areata, CREST syndrome (calcinosis, Raynaud's phenomenon, esophageal dysmotility, sclerodactyly, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, fibrosis of any organ or tissue, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Leishmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis. Other examples of autoimmune diseases may be disclosed elsewhere herein.

The following are non-limiting examples of cancers that can be diagnosed, treated, or monitored for treatment by the disclosed methods and compositions: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

Pharmaceutical compositions according to the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present disclosure to be administered can be determined by a physician with consideration of individual differences in age, weight, disease type, extent of disease, and condition of the patient (subject).

Typically, dosages of a compound according to the disclosure which may be administered to an animal range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. In some embodiments, the dosage of the compound will vary from about 1 mg to about 100 mg per kilogram of body weight of the animal. In some embodiments, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

Inhibiting Microglia-Derived IL-1β in Neovascular AMD

In some embodiments, the invention provides methods for treating or preventing neovascular AMD through inhibiting microglia-derived IL-1β. Accordingly, the invention provides inhibitors (e.g., antagonists) of IL-1β. In one embodiment, the inhibitor of the invention decreases the amount of IL-1β polypeptide, the amount of IL-1β mRNA, the amount of IL-1β activity, or a combination thereof.

It will be understood by one skilled in the art, based upon the disclosure provided herein, that a decrease in the level of IL-1β encompasses the decrease in the expression, including transcription, translation, or both. The skilled artisan will also appreciate, once armed with the teachings of the present invention, that a decrease in the level of IL-1β includes a decrease in the activity of IL-1β. Thus, decrease in the level or activity of IL-1β includes, but is not limited to, decreasing the amount of polypeptide of IL-1β, and decreasing transcription, translation, or both, of a nucleic acid encoding IL-1β; and it also includes decreasing any activity of IL-1β as well.

In one embodiment, the invention provides a generic concept for inhibiting IL-1β as an anti-tumor therapy. In one embodiment, the composition of the invention comprises an inhibitor of IL-1β. In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

One skilled in the art will appreciate, based on the disclosure provided herein, that one way to decrease the mRNA and/or protein levels of IL-1β in a cell is by reducing or inhibiting expression of the nucleic acid encoding IL-1β. Thus, the protein level of IL-1β in a cell can also be decreased using a molecule or compound that inhibits or reduces gene expression such as, for example, siRNA, an antisense molecule or a ribozyme. However, the invention should not be limited to these examples.

In one embodiment, siRNA is used to decrease the level of IL-1β. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of IL-1B at the protein level using RNAi technology.

In other related aspects, the invention includes an isolated nucleic acid encoding an inhibitor, wherein an inhibitor such as an siRNA or antisense molecule, inhibits IL-1β, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York) and as described elsewhere herein. In another aspect of the invention, IL-1β or a regulator thereof, can be inhibited by way of inactivating and/or sequestering one or more of IL-1β, or a regulator thereof. As such, inhibiting the effects of IL-1β can be accomplished by using a transdominant negative mutant.

In another aspect, the invention includes a vector comprising an siRNA or antisense polynucleotide. Preferably, the siRNA or antisense polynucleotide is capable of inhibiting the expression of IL-1β. The incorporation of a desired polynucleotide into a vector and the choice of vectors is well-known in the art as described in, for example, Sambrook et al., supra.

The siRNA or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

In one embodiment of the invention, an antisense nucleic acid sequence which is expressed by a plasmid vector is used to inhibit IL-1β. The antisense expressing vector is used to transfect a mammalian cell or the mammal itself, thereby causing reduced endogenous expression of IL-1β.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Compositions and methods for the synthesis and expression of antisense nucleic acids are as described elsewhere herein.

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit IL-1β. Ribozymes useful for inhibiting the expression of a target molecule may be designed by incorporating target sequences into the basic ribozyme structure which are complementary, for example, to the mRNA sequence of IL-1β of the present invention. Ribozymes targeting IL-1β may be synthesized using commercially available reagents (Applied Biosystems, Inc., Foster City, CA) or they may be genetically expressed from DNA encoding them.

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In another aspect of the invention, IL-1β can be inhibited by way of inactivating and/or sequestering IL-1β. As such, inhibiting the effects of IL-1β can be accomplished by using a transdominant negative mutant. Alternatively an antibody specific for IL-1β (e.g., an antagonist to IL-1B) may be used. In one embodiment, the antagonist is a protein and/or compound having the desirable property of interacting with a binding partner of IL-1β and thereby competing with the corresponding protein. In another embodiment, the antagonist is a protein and/or compound having the desirable property of interacting with IL-1β and thereby sequestering IL-1β.

As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, NY). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic affinity cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, NY).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, NY) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)2 fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

In some embodiments, the IL-1β inhibitor of the invention can be administered specifically to microglia using a targeting domain or a delivery vehicle comprising a targeting domain. Exemplary methods for cell-specific delivery of therapeutic molecules include, but are not limited to, the use of bispecific antibodies, lipid nanoparticles (LNP) and fusion peptides comprising targeting domains specific for binding to a marker of a specific cell type of interest (e.g., microglia.) Therefore, in some embodiments the invention provides methods for targeted administration of an IL-1β inhibitor to microglia. In some embodiments the invention provides methods for treating AMD by targeted administration of an IL-1β inhibitor to microglia.

### Pharmaceutical Compositions

The present invention includes pharmaceutical compositions comprising one or more inhibitors of IL-1β. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intratumoral, epidural, intracerebral, intracerebroventricular, or another route of administration. Other contemplated formulations include projected nanoparticles, liposomal preparations, lipid nanoparticle formations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Comparing CATCH to Other Single-Cell Clustering Approaches Using Splatter Synthetic Data

In order to benchmark CATCH against tools which can produce multigranular clusters, synthetic data generated by Splatter was used. Splatter simulates high dimensional non-linear single cell data by generating the expression for each gene from a gamma distribution and the count of each cell from a Poisson distribution, effectively allowing for the addition of increasing amounts of biological noise, drop out and variation, in data where ground truth is known (FIG. 3A). In this manner, data was generated where ground truth cluster labels are known and the performance of various implementations of diffusion condensation were tested against one another using adjusted rand index. Using this process, 40 different datasets were simulated with increasing amounts of drop out or variation, using adjusted rand index to compare diffusion condensation against louvain, leiden and Seurat. With each of these datasets, the CATCH framework was followed: first the condensation homology was computed and visualized (FIG. 3B), before topological activity analysis was performed to identify the top four most persistent granularities (FIG. 3C) and then finally the adjusted rand index, a common measure for determining clustering accuracy against a set of ground truth cluster labels (FIG. 3D) was computed, keeping the highest score for the comparisons. While naturally louvain and leiden are multigranular clustering techniques, they have been implemented in scanpy as single resolution strategies. In order to produce four different resolutions of clusters for each algorithm, the approach was run multiple times with different ‘resolutions,’ a parameter which allows the user to control the granularity of clusters. Seurat gives no option to control resolution, so only the returned set of cluster labels was used in the comparison.

### Example 2: Comparing CATCH to Other Single-Cell Clustering Approaches Using Real Single Cell Data

In order to benchmark CATCH against other single cell clustering tools on real single cell data, datasets in which cell types are established by an expert scientist familiar with the biological system were used (Wagner, D. E. et al., 2018, Science 360, 981-987; Aghaeepour, N. et al., 2013, Nature methods 10, 228-238). First, real single cell transcriptomic data generated from a developing zebrafish with ground cluster truth cell types identified by a known expert in the field was used (Wagner, D. E. et al., 2018, Science 360, 981-987). These cluster labels were organized into multigranular cluster labels by first aggregating 18 cell types found into four tissue types before aggregating those into three germ layers. The top four most persistent CATCH granularities were compared against multigranular clusters computed using louvain and leiden, again tuning the resolution parameter to produce ten different cluster labels. Finally, to show the generalizability of the CATCH approach, the CATCH framework was tested against louvain, leiden and FlowSOM (Van Gassen, S. et al., 2015, Cytometry Part A 87, 636-645) on flow cytometry data using the FlowCAP I ND dataset (Aghaeepour, N. et al., 2013, Nature methods 10, 228-238). The FlowCAP I ND dataset contains 10-dimensional data from 30 samples with approximately 40,000 cells per sample and a total of over 1.3 million cells post filtering. The clustering task is to detect 7 manually gated populations. Further details on the dataset are available from the FlowCAP website (flowcap.flowsite.org/). In this comparison, FlowSOM was included as it is the leading tool for identifying single cell populations from flow cytometry data. As done previously, multiple granularities of clusters were computed using leiden, louvain and CATCH. For FlowSOM, 7 clusters were asked for as this matches the granularity of clusters in the data.

### Example 3: Diffusion-Topological Analysis of Single-Cell Hierarchy Reveals Inflammatory Interactions Driving Macular Degeneration

This study describes the development of a novel topologically-inspired machine learning suite of tools called Cellular Analysis with Topology and Condensation Homology (CATCH). At the center of this framework is diffusion condensation. Diffusion condensation is a recently proposed data-diffusion based dynamic process for continuous graining of data through a deep cascade of graph diffusion filters. The algorithm iteratively pulls points towards the weighted average of their graph diffusion neighbors, slowly eliminating variation. When data points come close to each other, the points were merged to create a new cluster. This process reveals clusters across granularities before converging all data to a single point. Recognizing the similarity of diffusion condensation to computational homology from the field of topological data analysis, a suite of tools was developed around this coarse graining process. While traditional computational homology operates in ambient space, effectively merging points based on a growing distance threshold, diffusion condensation has the advantage of operating on the manifold through powering the diffusion filter. By measuring the rate of creation and destruction of clusters during the condensation process, stable granularities were identified with low topological activity for downstream analysis. A single-cell level enrichment analysis known as MELD (Burkhardt, D. B. et al., 2020, bioRxiv) was used to identify disease-enriched populations of cells within these salient levels of granularity. Furthermore, an automated characterization of these cell types was created by using the natural cluster centroid formed during the condensation process. Finally, by leveraging the complete cellular hierarchy as identified by diffusion condensation, pathogenic and healthy clusters of cells were efficiently compared via condensed transport to identify differentially expressed genes. This approach rapidly approximates Wasserstein distance between populations of interest, creating rich signatures of disease.

CATCH was applied to one of the most heterogeneous tissues in the human body, the retina, affected by a complex degenerative disease, age-related macular degeneration (AMD). AMD is a progressive disease of the retina that affects 196 million individuals worldwide (Wong, W. L. et al., 2014, The Lancet Global Health 2, e106-e116). Similar to other degenerative diseases of the central nervous system, such as Alzheimer's disease (AD) and progressive multiple sclerosis (MS), AMD can be categorized into distinct stages. Initially, in the early, ‘dry’ stage of AMD, focal deposits of extracellular lipid-rich debris known as drusen accumulate below the retinal pigment epithelium leading to activation of glia (Mitchell, P. et al., 2018, Lancet 392, 1147-1159). In advanced, ‘neovascular’ AMD, angiogenesis and fibrosis driven by vascular endothelial growth factor (VEGF) cause photoreceptor loss and a progressive decline in central visual acuity (Bird, A. C. et al., 1995, The International ARM Epidemiological Study Group. Surv Ophthalmol 39, 367-374). While the stages of AMD have been extensively clinically characterized, several open questions remain: What cell types are most affected in each phase of the disease? What cell types are responsible for driving disease progression from the dry to neovascular phase? What are the upstream regulators of VEGF production in late-stage disease? As the retina forms one of the most complex and heterogeneous tissues in the human body with many abundant as well as extremely rare cell types with disparate functions, learning and analyzing cellular subsets in a healthy retina has required either extensive sequencing effort or a priori targeted enrichment of cell types of interest (Peng, Y.-R. et al., 2019, Cell 176, 1222-1237.e22; Menon, M. et al., 2019, Nat. Commun. 10, 4902; Shekhar, K. et al., 2016, Cell 166, 1308-1323.e30). Analyzing retinas affected by AMD in addition to healthy samples adds further complexity, necessitating sophisticated machine learning methods to identify, characterize and compare healthy and pathogenic populations of cells.

To identify cell types responsible for driving AMD disease progression across the cellular hierarchy in an unbiased manner using CATCH, massively parallel microfluidics-based single-nucleus RNA sequencing (snRNA-seq) was performed on the retinal macula from healthy donors, and patient donors affected by the dry and neovascular stages of AMD. With the computational approach and dataset, two populations of glia were identified, one microglial subset and one astrocyte subset, enriched in the early phase of dry AMD. These subsets were characterized by signatures of phagocytosis, lipid metabolism, and lysosomal functions. By reapplying CATCH to AD and MS single-cell data sets, the same signatures were identified in early phases of multiple neurodegenerative diseases, indicating a common mechanism for glial activation in the early phase of neurodegeneration (Mathys, H. et al., 2019, Nature 570, 332-337; Schirmer, L. et al., 2019, Nature 573, 75-82). Finally, these microglia and astrocyte expression signatures were validated in human retinal and brain tissue. In late stage, neovascular AMD, CATCH identified an inflammasome expression signature in microglia as well as proangiogenic signature in astrocytes. Through further computational receptor ligand interaction analysis, a key signaling axis was identified between microglia-derived IL-1β and pro-angiogenic astrocytes, the driver of neovascularization and photoreceptor loss in advanced disease (Bird, A. C. et al., 1995, The International ARM Epidemiological Study Group. Surv Ophthalmol 39, 367-374). Through a combination of human induced pluripotent stem cell (iPSC)-derived astrocyte stimulation assays, in vivo mouse experiments, and analysis of postmortem human AMD retinal samples, this pro-angiogenic microglial-astrocyte axis mediated by IL-1β in late-stage neovascular AMD was validated.

The CATCH algorithm identified and characterized specific subpopulations of microglia and astrocytes enriched in the early stage of AMD displaying activation signatures related to phagocytosis, lipid metabolism, and lysosomal function. Similar populations were found in analyses of previously published AD and MS single-cell data. While initial inciting events likely differ between neurodegenerative conditions, lipid-rich extracellular plaques play a prominent role in each condition. It is likely that glial cells coordinate clearance of extracellular debris and, in turn, become activated. While the initial phagocytic clearance may be beneficial, glial activation has been shown to play a role in degeneration in AMD, AD, and MS. In later stages of disease, this shared landscape is largely replaced by a disease-specific stress state. In advanced neovascular AMD, CATCH identified a microglia inflammasome-related signature that drives proangiogenic astrocyte polarization and pathologic neovascularization. Microglial inflammasome activation and subsequent IL-1β release could be mediated by a variety of signaling sensors. The NLRP3 sensor may be activated in response to a variety of stress signals, including extended lipid exposure or prolonged hypoxia, and has been previously implicated as a microglial driver of neurodegenerative immunopathology, making it a likely candidate (Heneka, M. T et al., 2018, Nature Reviews Neuroscience 19, 610-621).

This set of analyses has clear implications for potential therapeutics for AMD. Currently, anti-VEGF therapy is the primary intervention approved to treat AMD and is only effective in the most advanced stage of disease. The unbiased topological analysis not only identified the cell-type specificity of VEGFA expression but also identified pathogenic signaling interactions that promote AMD disease progression. Currently, therapies that inhibit IL-1β are available and used in clinical practice for the treatment of other diseases. Inhibiting microglia-derived IL-1β in neovascular AMD could provide therapeutic benefit, preventing further neovascularization in advanced patients, or even preventing neovascularization before it begins in patients with earlier stages of disease.

The application of CATCH to AMD highlights its capability for learning the placement of important rare cellular states in the complex hierarchy of retinal tissue. By observing the differences in the geometry underlying the observed transcriptional states in health and multiple phases of disease, specific predictions can be made about the cell types and signaling pathways that drive disease progression.

This study offers both a framework for identifying disease-affected cellular populations, disease signatures, and causal mechanisms from complex single cell data as well as key insights into the disease-phase specific drivers of age-related macular degeneration.

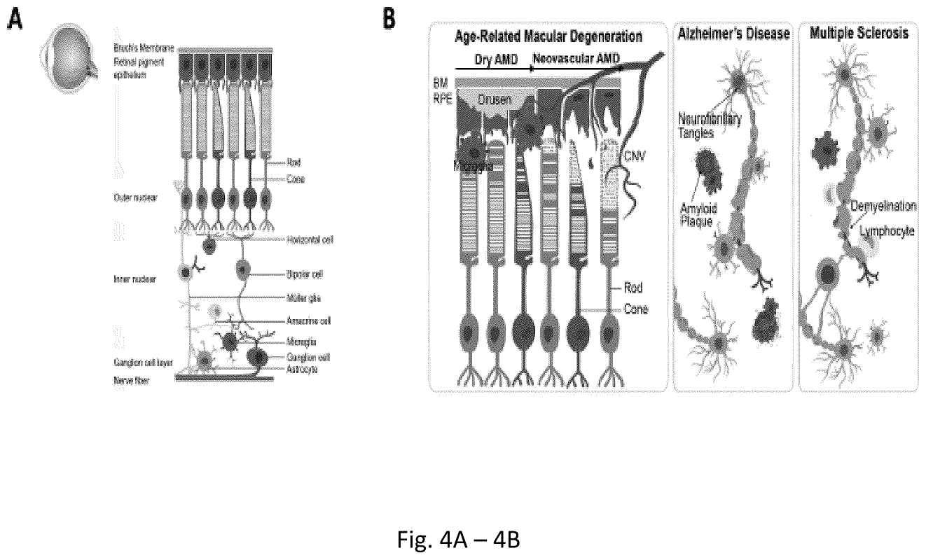

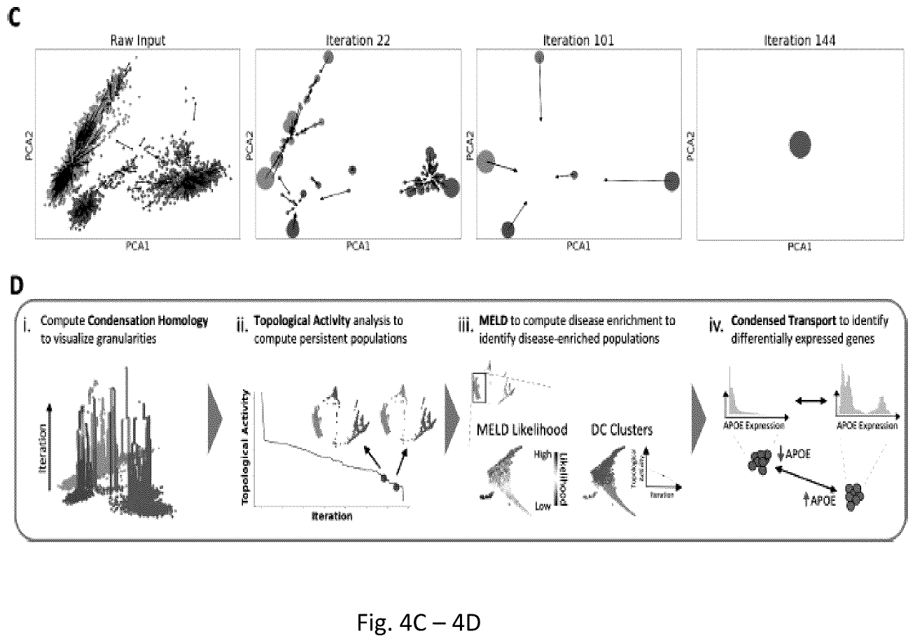

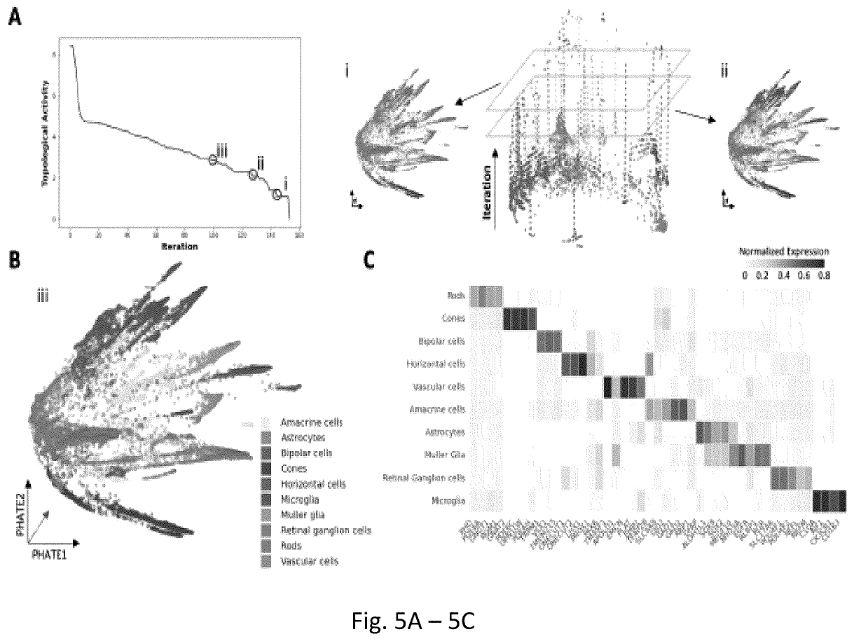

Heterogeneous tissues are made up of cells with diverse functional and transcriptomic states, creating a complex hierarchy which can be difficult to learn without apriori knowledge of cell-to-cell relations across granularities. The retina, for instance, is one of the most complex tissues in the human body, containing many different functional layers, distinct strata to the structure of the blood supply and glial organization, and is occupied by a highly diverse set of cell types and states (FIG. 4A). These cellular phenotypes are perturbed in disease conditions like AMD, adding further complexity to the cellular hierarchy (FIG. 4B). Differences between diseased and normal tissue range from systemic differences in large populations of cells to small shifts in rarer cell types. Thus, identifying meaningful granularities of the cellular hierarchy at which to analyze pathogenic populations becomes critical. The CATCH framework includes a set of tools to learn and analyze the cellular hierarchy to identify pathogenic transcriptomic states and signatures of disease: 1. Learning the cellular hierarchy through a coarse graining process called manifold-intrinsic diffusion condensation (FIG. 4C) 2. Visualizing condensation homology to study the computed cellular hierarchy (FIG. 4D-i) 3. Identifying meaningful granularities of the cellular hierarchy through topological activity analysis (FIG. 4D-ii) 4. Identifying clusters that isolate cells found disproportionately in pathogenic samples using the single cell enrichment analysis method MELD (FIG. 4D-iii) (Burkhardt, D. B. et al., 2020, bioRxiv) 5. Characterizing differentially expressed genes in pathogenic populations of cells using condensed transport (FIG. 4D-iv)

The materials and methods employed in these experiments are now described.

CATCH Improves Ability to Find Shared Gene Signatures

Typically, disease associated signatures are uncovered by performing differential expression analysis between cells from the disease condition and cells from the healthy condition at the resolution of cell type. For instance, microglia would be separated into two groups based on condition of origin, either disease or healthy, which would then be compared. Comparing cellular states identified with CATCH for different conditions of origin can identify transcriptomic differences and similarities more rigorously. To illustrate this point, differential expression analysis was performed between microglia based on their condition of origin across all three diseases. After setting significance a cutoff for the Condensed Transport (cutoff of 0.044, 0.130 and 0.164 for AMD, MS and AD respectively, representing the top 10% of genes as significant), significantly enriched genes were identified in the early or acute active phase of each disease (FIG. 12A). However, across all comparisons, significantly fewer differentially expressed genes were identified in this cell type analysis (135, 68 and 416) than with the CATCH pipeline (618, 795 and 1551 for AMD, AD and MS respectively), indicating that the identification of pathogenic cellular subtypes with CATCH before comparison increases the ability to detect differentially expressed genes. In cross-disease comparisons among early-stage neurodegenerative microglia, only 17 common genes were found, significantly less than the 168 common genes found with the CATCH pipeline. Of the common genes, only half of the activation signature was found (APOE, B2M, FTH1, FTL, SPP1). Similar to the coarse-grained microglial comparison, the strength of the topological approach was compared in astrocytes. Comparing astrocyte states based on condition of origin with previously set significance cut offs (0.034, 0.061, and 0.071 for AMD, AD and MS respectively, all again reflecting a top 10% of genes as significant), significantly fewer enriched genes (221, 271, and 886) were identified than were found with the CATCH topological analysis (1444, 680, and 2278 genes for AMD, AD and MS respectively) (FIG. 12B). In the cell type level analysis, only 28 common genes were found, significantly less than the 630 common genes found with the CATCH pipeline. Of the common genes, only half of the activation signature was found (AQP4, CD81, CRYAB, GFAP). Collectively, these comparisons reveal the sensitivity of this discovery pipeline for finding gene signatures and biologically meaningful relationships among noisy data in gene expression space.

Single-Nucleus AMD RNA Sequencing and Pre-Processing

snRNA-seq data from macular samples, were processed according to the following steps. Sample demultiplexing and read alignment to the NCBI reference pre-mRNA GRCh38 was completed to map reads to both unspliced pre-mRNA and mature mRNA transcripts using CellRanger version 3.1.0. Gene and cell matrices from retinas with dry AMD (n=3), neovascular AMD (n=8), and healthy controls (n=6) were then combined into a single file. Prefiltering was performed using parameters in scprep (v1.0.3, github.com/KrishnaswamyLab/scprep). Cells that contained at least 1400 unique transcripts were kept for further analysis to generate a cell by gene matrix containing 50,498 cells. Normalization was performed using default parameters with L1 normalization, adjusting total library side of each cell to 1000. Any cell with greater than 200 normalized counts of mitochondrial mRNA was removed. Batch correction was performed using Harmony (github.com/immunogenomics/harmony) to align batch effects introduced by sequencing batch, postmortem interval, sample acquisition location and 10× sequencing chemistry (Korsunsky, I. et al., 2019, Nature Methods 16, 1289-1296). Raw and processed data files for human snRNA-seq data will be available for download through GEO under an accession number to be assigned with no restrictions on data availability.

Single-Nucleus AD and MS RNA Sequencing Pre-Processing

snRNA-seq data for AD and MS was acquired from published sources (Mathys, H. et al., 2019, Nature 570, 332-337; Schirmer, L. et al., 2019, Nature 573, 75-82). Cells that contained at least 1000 unique transcripts were kept for further analysis to generate a cell by gene matrix for each disease. Normalization was performed using scprep default parameters with L1 normalization, adjusting total library side of each cell to 1000. Any cell with greater than 200 normalized counts of mitochondrial mRNA was removed. Batch correction was performed on MS data using Harmony (github.com/immunogenomics/harmony) to align batch effects introduced by sequencing batch, capture batch and sex.

Cell Type Identification with CATCH

All cell types were identified by performing topological activity analysis on the diffusion condensation calculated condensation homology. In order to identify cell types, resolutions with no topological activity were identified which partitioned the cellular state space well and assigned each cluster to a cell type based on cell type specific marker genes.

Interaction Analysis:

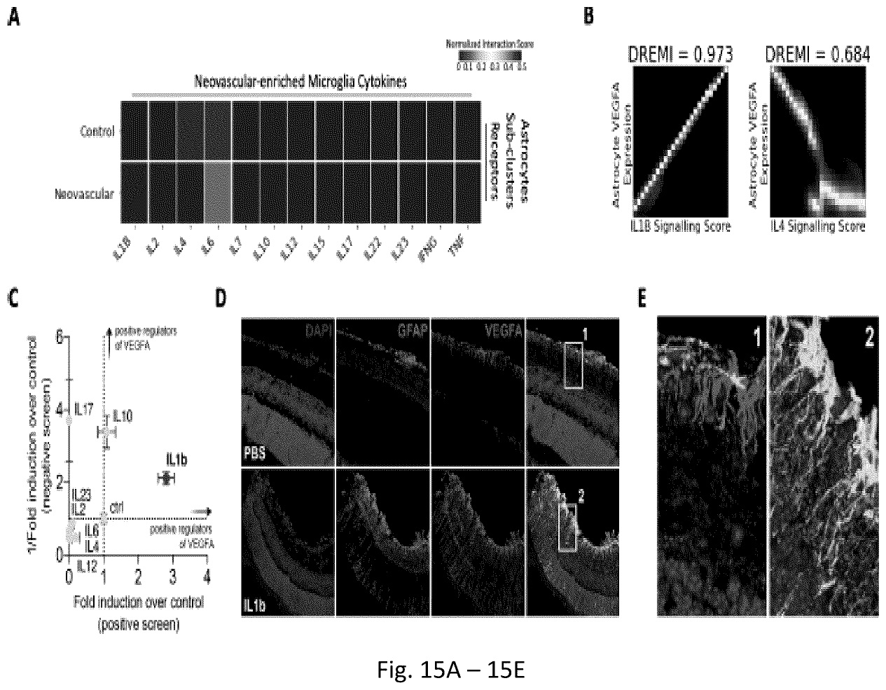

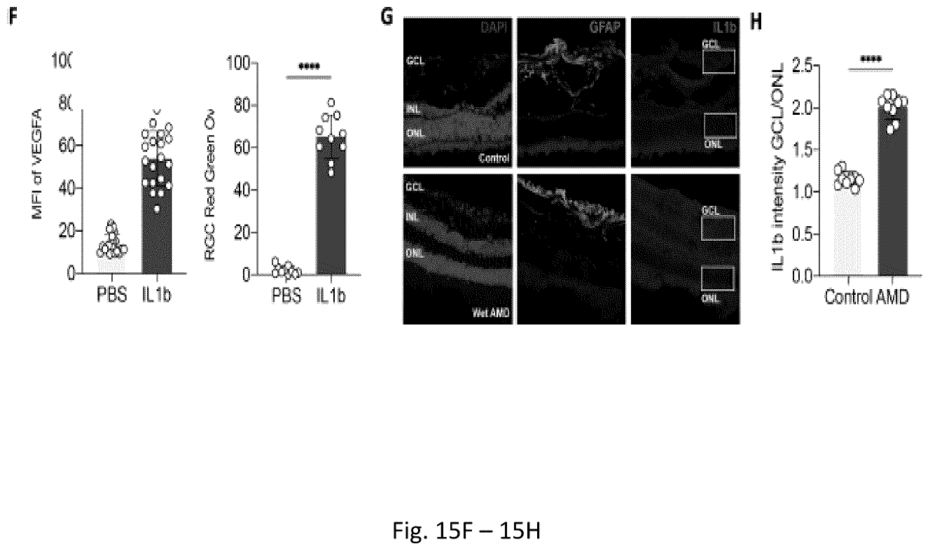

Cell-cell ligand-receptor analysis was conducted on pre-processed snRNA expression data using the CellPhoneDB python package (github.com/Teichlab/cellphonedb, v2.1.4) (Efremova, M et al., 2020, Nature Protocols 15, 1484-1506). Before conducting analysis, the package database of 834 curated ligand-receptor combinations and multi-unit protein complexes was supplemented with 2557 ligand-receptor interactions found in the celltalker database (github.com/arc85/celltalker) (Ramilowski, J. A. et al., 2015, Nature Communications 6). The in-built database-generate function was utilized to update the existing database. A comprehensive user-generated database was invoked in each run of the CellPhoneDB statistical-analysis command function. CellPhoneDB interaction maps were computed on differing inputs. First, disease phase enriched microglia and astrocytes with subcluster identity were run to identify signaling interactions between astrocyte and microglial activation states (FIG. 15B). The number of permutations was set to 2,000 and p-value threshold was set to 0.01.

Human Tissues

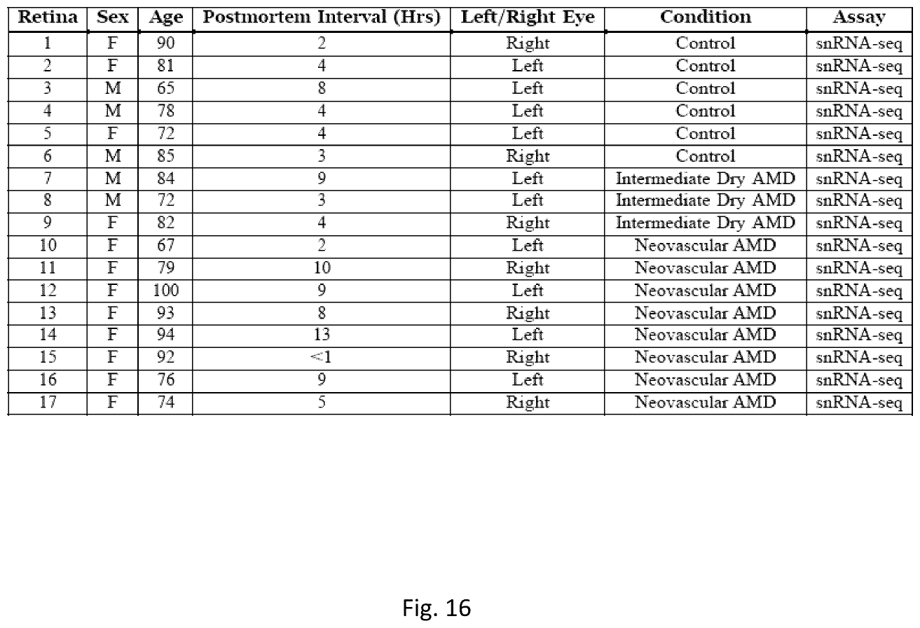

Postmortem eyes for the Chromium Single Cell 3′ assay (n=17) and medical records containing AMD disease stage were obtained from Advancing Sight Network (Alabama), Lions Gift of Sight Eye Bank (Minnesota), or the Yale Department of Pathology with a maximum post-mortem interval of 13 hours. Globes were examined for retinal disease by an ophthalmologist (B.P.H.) prior to dissection and dissociation of the samples. Retina for snRNA-seq was obtained from the unrelated human post-mortem donors that included normal, intermediate dry on AREDS2, and neovascular AMD stages (FIG. 16). For each sample the macula, which is the region of the retina responsible for central vision and affected most severely by AMD pathology, was profiled. Three intermediate AMD samples were identified from patients taking the AREDS2 eye vitamin and mineral supplement with drusen, a pathologic sign associated with the intermediate dry stage of the disease. Eight postmortem AMD samples had neovascularization in the advanced stage of the disease. Normal donors had no history of retinal disease.

Retinal Dissection and Solation of Nuclei from Frozen Retinal Tissue

Globes were placed in RNAlater (ThermoFisher) and transported on ice. Trephine punches (6 mm diameter) were used to isolate samples from the macula in the central retina, located away from the optic disc and major arterioles. For each punch of tissue, the retina was mechanically separated from the underlying retinal pigment epithelium-choroid, snap-frozen on dry ice and stored at −80° C. Nuclei were isolated and purified using the Nuclei EZ Prep Nuclei Isolation Kit (Sigma), following the manufacturer's protocol, with some modification. All procedures were carried out on ice or at 4° C. Briefly, frozen retinal tissue was subjected to dounce homogenization (25 times with pestle A followed by 25 times with tight pestle B) using the KIMBLE Dounce Tissue Grinder Set (Sigma) in 2 ml EZ Lysis buffer. The sample was transferred to a 15 ml tube with an additional 2 ml EZ lysis buffer and incubated on ice for 5 min. Following incubation, the sample was centrifuged at 500×g, 5 min at 4° C. Supernatants were discarded, and the isolated nuclei were resuspended in 4 ml EZ lysis buffer, incubated for 5 min on ice and centrifuged at 500×g for 5 min at 4° C. Next, the nuclei were washed with 4 ml ice-cold Nuclei Suspension Buffer (1×PBS containing 0.01% BSA and 0.1% RNAse inhibitor), resuspended in 1 ml Nuclei EZ Storage buffer and passed through a 40 μM nylon cell strainer. The nuclei suspensions were counted with trypan blue prior to loading on the microfluidics platform.

Droplet-Based Microfluids snRNA-Seq

Isolated nuclei from each macular sample was processed through microfluidics-based single nuclear RNA-seq. Single-cell libraries were prepared using the Chromium 3′ v2 and v3 platforms (10× Genomics) following the manufacturer's protocol. Briefly, single nuclei were partitioned into Gel beads in Emulsion in the 10× Chromium Controller instrument followed by lysis and barcoded reverse transcription of RNA, amplification, shearing and 5′ adapter and sample index attachment. On average, 7000 nuclei were loaded on each channel that resulted in the recovery of 4000 nuclei. Libraries were sequenced on the Illumina NextSeq 500 platform. After quality control preprocessing, snRNA-seq profiles were used in subsequent analyses. This dataset was corrected for batch effects across samples using the Harmony algorithm (Korsunsky, I. et al., 2019, Nature Methods 16, 1289-1296).

In Situ RNA Hybridization and Immunofluorescence

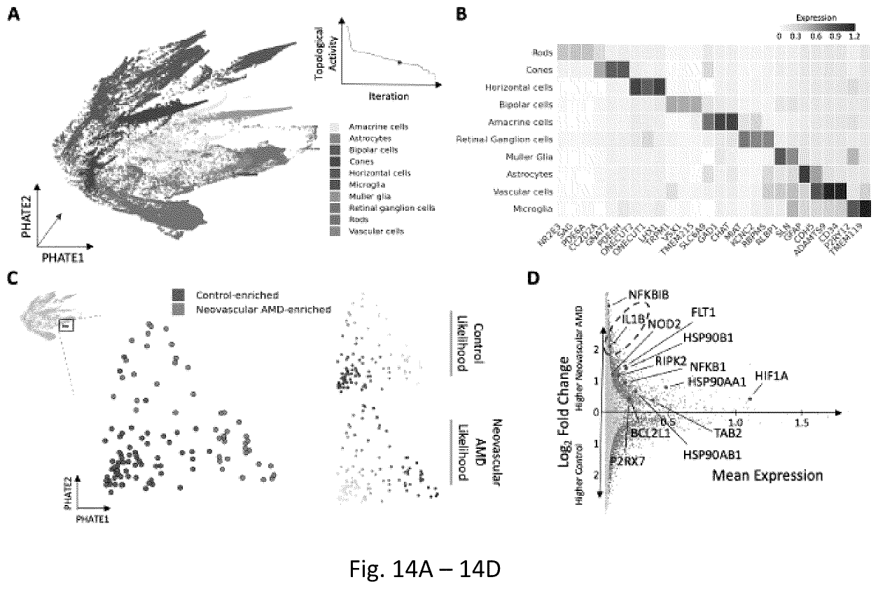

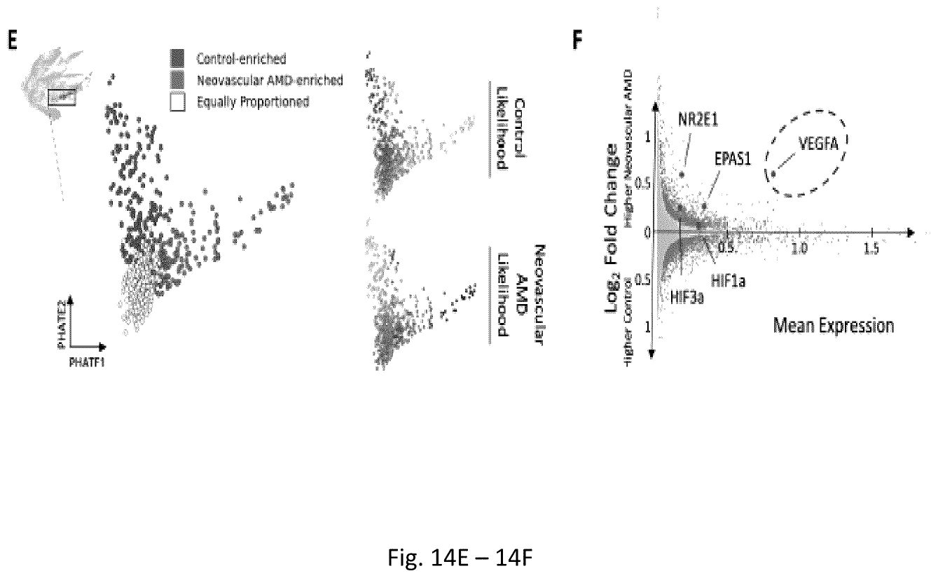

To validate the gene expression differences, in situ hybridization was performed using RNAscope Multiplex Fluorescent V2 Assay (Advanced Cell Diagnostics, Hayward, CA, USA). Macula dissected from whole human globes were fixed in 4% paraformaldehyde (PFA) at 4.0 overnight. Tissues were sequentially dehydrated with 15% sucrose, then 30% sucrose before embedding in OCT, and frozen on dry ice. OCT molds were sectioned at 10 μm thickness. RNA in situ hybridization was performed according to the manufacturer's protocol. Briefly, fixed frozen sections were baked at 60.0 for 1 hr prior to incubation in 4% PFA for 10 mins and protease digestion pretreatment. Target probes were hybridized to an HRP-based temperature sensitive signal amplification system, followed by color development. Housekeeping genes POLR2A, PPIB, and UBC were used as internal-control mRNA (FIG. 14); if probes for these mRNAs were not visualized, the sample was regarded as not available for gene expression study. The probes used include APOE, TYROBP, B2M, VEGFA, and HIF1A (Advanced Cell Diagnostics, Hayward, CA, USA). The slides were counterstained with DAPI during immunofluorescence protocol (see below). Positive staining was determined by fluorescent punctate dots in the appropriate channels in the nucleus and/or cytoplasm. Following RNA in situ hybridization protocol, fixed frozen sections were blocked with animal serum and incubated overnight at 4.0 with primary antibodies (see antibody segment below). Secondary antibody incubation was for 1 hr at room temperature and cell nuclei were counterstained with DAPI. Images were captured immediately using a confocal microscope (Zeiss LSM800, Jena, Germany). The following antibodies against human antigens were used: GFAP (1:500, MA5-12023, Invitrogen) and Iba1 (1:500, 019-19741, Fujifilm). Antibodies were visualized with Alexa Fluor 488 (1:200, A-11001/A-21208, Invitrogen).

Mice

Four- to-eight-week-old mixed sex C57BL/6 mice were purchased from the National Cancer Institute and subsequently bred and housed at Yale University. All procedures used in this study (sex-matched, age-matched) complied with federal guidelines and the institutional policies of the Yale School of Medicine Animal Care and Use Committee.

Cells

IPSC-derived astrocyte cells were purchased from Brainxell.com (Brainxell, Madison Wisconsin). Cells were cultured according to provider's guidelines using 1:1 DMEM/F12 and Neurobasal medium with N₂ supplement (1×), Glutamax (0.5 mM), Astrocyte supplement (1×), Fetal bovine serum (1%).

Cell Culture

IPSC-derived astrocyte cells were cultured to a fully differentiated state before cytokine stimulation. Cytokines, (IL-1β, IL2, IL4, IL6, IL7, IL10, IL12, IL15, IL17, IL22, IL23, IFNG, TNF) were all purchased from PeproTech.com (Peprotech, Cranbury, NJ). For single cytokine stimulation, cells were stimulated with each cytokine at a concentration of 100 ng/mL for 24 hours. For combinatorial cytokine stimulation, cocktail of all cytokines minus cytokine of interest was made with each cytokine concentration at 50 ng/mL. Cells were stimulated for 24 hours before media was collected. Collected media was centrifuged at 1000×g to remove any cells and debris before performing an ELISA.

Enzyme-Linked Immunosorbent Assay

Enzyme-linked immunosorbent assay (ELISA) was performed using a mouse VEGF-A ELISA Kit (Cusabio LLC) following the manufacturer's instructions.

Intravitreal Injection

Mice were anaesthetized using a mixture of ketamine (50 mg/kg) and xylazine (5 mg/kg), injected intraperitoneally. Mice eyes were sterilized using betadine. A small hole was made at the lateral aspect of the limbus was made using a 33 gauge insulin syringe. Using a blunt end Hamilton syringe, 1 μl of PBS or IL-1β (100 ng) was injected at a 45 degree angle at the limbus intravitreally. Once the infusion was finished, syringe was left in place for a minute before removal of the syringe. Injection site was washed with sterile PBS and puralube vet ointment was applied to the eyes. Mice were monitored until full recovery.

Mice Tissue Processing and Microscopy

Retinas were dissected, fixed in 2% PFA for one hour and immediately processed in a blocking solution (10% normal donkey serum, 1% bovine serum albumin, 0.3% PBS-Triton X-100) for overnight incubation at 4° C. For retina sections, primary antibodies were incubated overnight at 4° C., then washed five times at room temperature in PBS and 0.5% Triton X-100, before incubation with a fluoro-conjugated secondary antibody diluted in PBS and 0.5% Triton X-100 for 2 hours in room temperature. Sections were washed five times at room temperature, stained with DAPI and mounted before imaging. Confocal images were taken on a Leica SP8 microscope. Quantitative analysis was performed using either FIJI or ImageJ image-processing software (NIH or Bethesda) or Imaris 8 software (Oxford Instruments).

The results of the experiments are now described.

Manifold-Intrinsic Diffusion Condensation Learns Cellular Hierarchy from Single-Cell Transcriptomic Data Through a Deep Cascade of t-Step Diffusion Filters

Diffusion condensation was first proposed in (Brugnone, N. et al., 2019, IEEE International Conference on Big Data (Big Data), 2624-2633). This process was built upon the data diffusion framework which learns the geometry of a dataset by simulating random walks over the data graph by calculating a diffusion operator (Coifman, R. R. et al., 2006, Applied and computational harmonic analysis 21, 5-30). While in the original work, the diffusion operator was eigendecomposed to visualize the data (Coifman, R. R. et al., 2006, Applied and computational harmonic analysis 21, 5-30), in other work, this diffusion operator has been applied directly back to the input data as a graph filter to remove noise from the input dataset (van Dijk, D. et al., 2018, Cell 174, 716-729.e27). In fact, this diffusion process has been used widely in single-cell analysis as it effectively learns the non-linear geometry, or manifold, of complex transcriptomic datasets in many contexts (van Dijk, D. et al., 2018, Cell 174, 716-729.e27; Haghverdi, L. et al., 2015, Bioinformatics 31, 2989-2998; Moon, K. R. et al., 2019, Nature Biotechnology 37, 1482-1492). Here, diffusion condensation is built upon to understand the hierarchy of complex single cell datasets.

Algorithm 1: Manifold-intrinsic Diffusion Condensation

Input: Cell-by-PC data matrix X, initial kernel bandwidth parameter εo and merge threshold ζ
Output: cluster labels by iteration

| | |
|---|---|
| 1: | Xₒ X, i 0 |
| 2: | while Xᵢ > 1 do |
| 3: | Merge data points a,b if Xᵢ(a) Xᵢ(b) ₂ < ζ, where Xᵢ(a) is the a-th row of Xᵢ |
| 4: | Update the cluster assignment for each original data point based on merging |
| 5: | Dᵢ ← compute pairwise distance matrix from Xᵢ |
| 6: | Kᵢ ← kernel affinity(Dᵢ, εᵢ) |
| 7: | Pᵢ ← row normalize Kᵢ to get a Markov transition matrix (diffusion operator) |
| 8: | tᵢ ← spectral entropy of Pᵢ |
| 9: | Xᵢ₊₁ ← Ptᵢ Xᵢ |
| 10: | εᵢ₊₁ ← update(εᵢ) |
| 11: | i = i + 1 |
| 12: | end while |

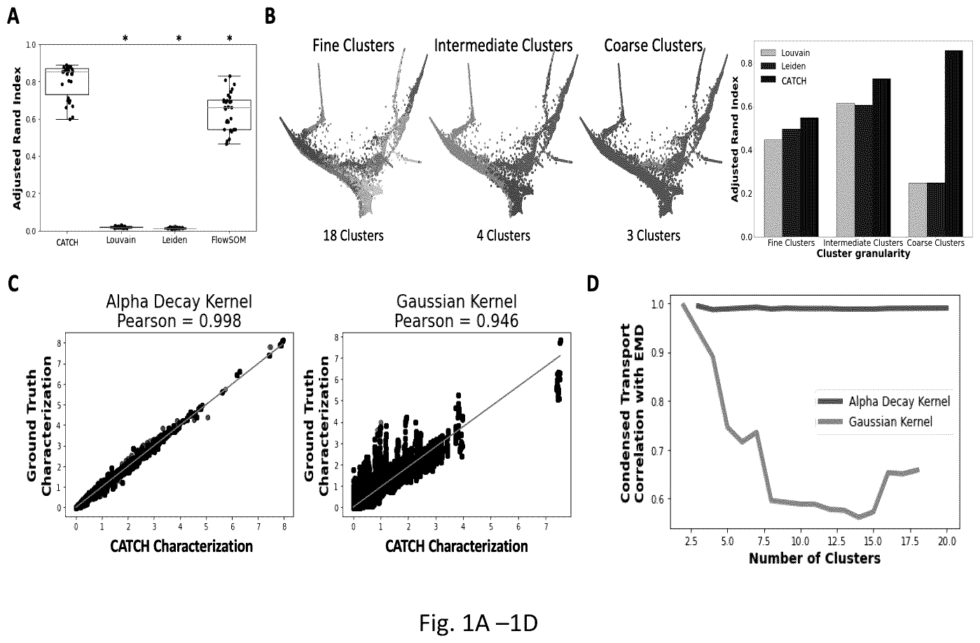

The manifold-intrinsic diffusion condensation algorithm takes a cell-by-principle component matrix X (typically first 50 components) and computes a diffusion operator P, representing the probability distribution of transitioning from one cell to another in a single step using a α-decay kernel function with fixed bandwidth ε (Alg. 1: Steps 5-7). While other manifold learning techniques abstract the data to a point where derived manifold-intrinsic features have an unclear relationship with gene expression, this approach learns the manifold while working in principle components, which have a clear relationship with genes. By using the principle components as the substrate for condensation, it is easy to characterize clusters and perform differential expression analysis in gene expression space in downstream analysis. Another key improvement made in the condensation algorithm is raising P to the power of t (rather than 1 as in (Brugnone, N. et al., 2019, IEEE International Conference on Big Data (Big Data), 2624-2633)), simulating a t-step random walk over the data. This approach adaptively denoises and refines these transition probabilities across iterations such that transitions occur on the data manifold (Coifman, R. R. et al., 2006, Applied and computational harmonic analysis 21, 5-30; van Dijk, D. et al., 2018, Cell 174, 716-729.e27; Moon, K. R. et al., 2019, Nature Biotechnology 37, 1482-1492). This t-step diffusion operator Pt are applied to the input data, acting as a manifold-intrinsic diffusion filter, effectively replacing the coordinates of a point with the weighted average of its t-step diffusion neighbors. The values of t computed across iterations is tracked and an ablation study is performed to show the necessity of adaptively tuning t in each iteration of the manifold-intrinsic diffusion condensation (FIG. 2A-B). See Alg. 1 for pseudocode of this algorithm. When the distance between two cells falls below a distance threshold c, cells are merged together, denoting them as belonging to the same cluster going forward (Alg. 1: Steps 3,4). This process is then repeated iteratively until all cells have collapsed to a single cluster. This merging step, implemented in the manifold-intrinsic diffusion condensation approach, allows for the fast computation of the cellular hierarchy during coarse graining. When applying this manifold-intrinsic diffusion condensation process to single-cell transcriptomic data, it can be seen that cells condense to cluster centroids across iterations, efficiently and rigorously learning the hierarchy of single-cells (FIG. 1C). Finally, through scalable implementation tricks, such as diffusion operator landmarking and weighted random walks, diffusion condensation was allowed to scale to thousands of single cells (FIG. 2F) (Gigante, S. et al., 2019, 13th International conference on Sampling Theory and Applications).

Visualizing and Analyzing Condensation Homology with Topological Activity Analysis to Identify Meaningful Granularities for Downstream Analysis

Topological data analysis (TDA) is a powerful framework that learns and analyzes data across granularities. In TDA, one identifies related data points by identifying all pairs whose distance falls below a distance threshold δ in a distance matrix D. Any pair of points that falls below this threshold is deemed to be part of the same connected component or cluster. As δ increases, more cell pairs will be connected, quickly creating fewer connected components, or fewer larger clusters, at coarser granularities. In topological data analysis, persistent homology is a principled approach to track the connected components that are created and destroyed across a range of granularities (see Methods). While diffusion condensation learns the multigranular structure of data through a cascade of non-linear diffusion filtration approach instead of an increasing distance threshold, these approaches are intuitively related. Using persistent homology, a condensation homology, a technique to analyze the creation and destruction of clusters across consecutive iterations (Xi Xi+1) of the manifold-intrinsic diffusion condensation process, is defined. The condensation homology can be studied in multiple ways, both visually and computationally by employing aspects of TDA. First, the cellular hierarchy can be studied by visualizing the condensation homology, containing all merges across all granularities. As manifold-intrinsic diffusion condensation operators in PCA dimensions, this visualization is practically implemented by stacking the first two axes of Xi Xi+1 XI, creating a hierarchical tree that summarizes the cluster structure of the data across granularities (FIG. 1D-i). To identify these resolutions in a quantitative manner, a variation of the total persistence summary statistic often used to characterize topological activity in classical topological data analysis was employed (Chen, C. et al., 2011, IEEE International Conference on Computer Vision (ICCV), 423-430). In this analysis framework, the merging of points during the condensation process was summarized and each cluster was assigned a topological ‘prominence’ value known as persistence. Highly persistent components are taken to represent groups of cells that are similar in their transcriptional profile and distinct from other cells. These clusters, and their associated persistence values, are best represented using a ‘persistence barcode.’ This is a visualization consisting of horizontal bars of different lengths; each bar corresponds to one topological feature—a subgroup of cells in this case—while the length of each bar depicts the persistence of that feature, directly indicating to what extent the feature is prominent (Ghrist, R. 2008, Bulletin of the American Mathematical Society 45, 61-75). Assuming that the persistence barcode consists of a set of bars with end coordinates:=b1, . . . , bk, an activity curve was calculated A: ______ defined by A(i):=b b i, i.e., the number of topological features (cell clusters) that are active and independent at a given iteration i. This activity curve, first proposed by (Rieck, B. et al., 2020, Topological Methods in Data Analysis and Visualization V, 87-101) and implemented by (O'Bray, L. et al., 2021, 27th ACM SIGKDD International Conference on Knowledge Discovery & Data Mining (KDD), 1267-1275), allows identification of iterations of rapid condesation as well as iterations of relative inactivity through the gradient of A. Specifically, there is interest in contiguous segments in the preimage of ∂A/∂i=0, referred to as i-segments. The length of an i-segment is the number of iterations for which there is no change in topological activity. Thus, the number of iterations for which ∂A/∂i=0 provides a principled way of selecting meaningful condensation granularities computed by the diffusion condensation process. Inspired by the nomenclature of persistent homology, the length of a i-segment of no topological activity is referred to as its persistence, meaning that the most persistent of such topological activity segments is being looked for. This is the first time that the condensation process has been linked to the field of topological data analysis (TDA), representing a merge between diffusion geometry and computational topology.

Identification of Disease-Enriched Populations in Conjunction with MELD

While analysis of condensation homology will identify populations of related cells in an unbiased and multigranular manner, it does not use condition of origin information to identify cellular populations that are enriched in disease conditions of interest. While cells from different disease conditions can be integrated in this analysis, cells of a certain pathogenic transcriptomic state may be over represented in a submanifold of a given cell type. By comparing the cells of a particular type directly to each other based on condition of origin, this enrichment information is diluted and lose important signal. In fact, identifying these pathogenic states and comparing them directly with clustering and differential expression tools has been shown to be a more powerful method to identifying condition-enriched cell states and expression signatures (Dann, E. et al., 2021, Nature Biotechnology; Burkhardt, D. B. et al., 2020, bioRxiv) To take condition-specific information into consideration, MELD was used to identify cellular populations that are enriched or depleted in different disease phases (Burkhardt, D. B. et al., 2020, bioRxiv). MELD is a manifold-geometry based method of computing a likelihood score for each cell, indicating whether it is more likely to be seen in the normal or diseased sample. Finding a clustering method that separates these condition-enriched groups is a difficult problem that needs to be performed to identify discrete cellular populations which can be thoroughly described. To rigorously identify cell populations with strong disease-specific enrichment signals, this cell-level MELD score was combined with information from the topological activity analysis, effectively identifying granularities in the condensation homology that isolates cellular states enriched in differing disease conditions. More specifically, levels of the hierarchy were identified which optimally separate cells using topological activity analysis. The MELD likelihood scores were then mapped onto these levels to identify granularities which identify clusters enriched for particular conditions (FIG. 1D-iii). Once enriched populations are isolated, these populations were characterized and identify signatures of disease were identified using condensed transport.

Automated Cluster Characterization Via Manifold-Intrinsic Diffusion Condensation

While identification of pathogenic cellular states is critical, biologists are more interested in what defines these populations. Most manifold learning methods visualize or cluster populations of interest, requiring further expensive computation to characterize cell populations and discover differentially expressed genes. As this approach continuously condenses the transcriptomic profiles of single cells to local cluster centriods in manifold space, at any iteration, the transcriptomic states of the condensed data can be extracted at no additional computational cost. To enhance this convergence to centroids, the diffusion condensation process was implemented with an α-decay kernel (FIG. 2C). This kernel more strongly thresholds the conversion of distances to affinities, closely resembling the box kernel, which accurately computes cluster centroids over the course of main point merges. It was proven that, under particular epsilon settings, if there are two cells, xa, xb, that merge to create new point xab, then this new point is the centroid of xa and xb (Proposition 1). Furthermore, since data points xa and xb are in fact aggregated cells as well, xab actually defines the cluster centroid of all cells underlying xa and xb in transcriptomic space (See Proposition 1 and proof in Methods). Since, manifold-intrinsic diffusion condensation operates in PC dimensions, the complete gene expression profile of cluster centroid xab can easily extracted by inverting the PC dimensions. This is not only proven to be mathematically true but also empirically true in practice (FIG. 1C).

Differential Expression Analysis Via Condensed Transport of Genes

Beyond cluster characterization, differential expression analysis is a critical method to identify signatures of pathogenic populations. Earth Mover's Distance (EMD), also known as ‘optimal transport’, typically manifested in 1D-Wasserstein distance, is a popular and established method to extract differentially expressed genes between clusters (van Dijk, D. et al., 2018, Cell 174, 716-729.e27; Nabavi, S. et al., 2015, Bioinformatics 32, 533-541; Wang, T. et al., 2017, IEEE International Conference on Bioinformatics and Biomedicine (BIBM), 202-207; Orlova, D. Y. et al., 2016, PLOS ONE 11, e0151859). EMD, however, is computationally expensive, as it computes an optimal mapping between points, running in O\tilde{ } (n3) time. Previously, tree-based implementations like FlowTree and QuadTree have been able to closely approximate ground truth Wasserstein distance while significantly improving runtime by constraining the transport of points through the branches of a hierarchical tree (Indyk, P. et al., 2003, 3rd International Workshop on Statistical and Computational Theories of Vision; Le, T et al., 2019, In Advances in neural information processing systems, 12304-12315; Zappia, L et al., 2017, Genome Biology 18). Since diffusion condensation too produces a tree embedding of the data, tree based transport was utilized for differential expression. Using this intuition, CATCH is able to rapidly perform differential expression analysis through a process called condensed transport of genes along the hierarchies generated by manifold-intrinsic diffusion condensation. Leveraging this approach's ability to summarize transcriptomic landscapes with the α-decay kernel, condensed transport uses multiple granularities of the cellular hierarchy to accurately approximate ground truth Wasserstein distance between genes and identify cluster-specific expression signatures (FIG. 1D-iv) (Le, T et al., 2019, In Advances in neural information processing systems, 12304-12315). This was proven to be true not only mathematically (See Proposition 2 and proof) but also empirically (FIG. 2D and FIG. 1F).

Comparison to Other Clustering Algorithms on Synthetic and Real Single Cell Data

This CATCH approach was benchmarked against existing clustering strategies applied to single cell data. Using a combination of 40 synthetic single cell datasets as well as real single cell and flow cytometry data, the clustering performance of diffusion condensation was compared against Louvain and Leiden, multigranular clustering techniques often applied to single cell data, as well as Seurat and FlowSOM, state-of-art methods for clustering single-cell transcriptomic and flow cytometry data respectively. Splatter is a simulator of realistic single cell data where ground truth cluster labels are known (Zappia, L et al., 2017, Genome Biology 18). Using these ground truth labels, increasingly noisy single cell datasets was generated with two different types of biological noise: variation and drop out (FIG. 1A). With each of these datasets, the CATCH framework was followed: computation and visualization of the condensation homology was done (FIG. 1B) before performing topological activity analysis to identify the top four most persistent granularities (FIG. 1C) and then finally computing adjusted rand index, a common measure for determining clustering accuracy against a set of ground truth cluster labels (FIG. 1D), keeping the highest score from these comparisons. Intriguingly, the most persistent population (iv), nearly always had the highest adjusted rand index score. Using this comparison approach, diffusion condensation was compared to Louvain, Leiden, and Seurat clustering algorithms across synthetic single cell datasets. For Louvain and leiden of the comparison approach, four different resolutions of clusters were computed and compared, keeping only the comparison which produced the highest adjusted rand index. Across both increasing levels of drop out and increasing amounts of variation, CATCH performed better than Louvain, Leiden, and Seurat clustering algorithms across 10 different simulations. As noise increased to 0.7 and 0.9 drop out and 0.3 and 0.4 variation, CATCH outperformed other approaches in a statistically significant manner (two-sided t-test between CATCH and each of the other clustering approaches at each iteration, p-value<0.01) (FIG. 1E). Next, CATCH was compared against Louvain and Leiden clustering approaches on real single cell data where multigranular clusters had been identified by an biological expert (Aghaeepour, N. et al., 2013, Nature methods 10, 228-238; Wagner, D. E. et al., 2018, Science 360, 981-987). First, real single cell transcriptomic data generated from a developing zebrafish with known cell type cluster ground truths was analyzed (Wagner, D. E. et al., 2018, Science 360, 981-987). These cluster labels were organized into multigranular cluster labels by first aggregating 18 cell types found in four tissue types before aggregating them into three germ layers. In this manner, ground truth cluster labels across granularities were produced. The top four most persistent CATCH granularities were compared against multigranular clusters computed using Louvain and Leiden, again tuning the resolution parameter to produce ten different cluster labels. At all granularities of ground truth cluster labels, CATCH out performed Louvain and Leiden despite more granularities being computed for the comparison approaches (FIG. 1B). Finally, as flow cytometry gating analysis has long been held as the gold standard for cell type identification and comparison, CATCH was compared to other clustering approaches on flow cytometry data, reasoning that ideal cell types can be difficult to identify even for an expert in single cell transcriptomic data. Using 1.3 million cells generated from 30 patients, the performance of CATCH was compared to Louvain, Leiden and the flow cytometry clustering gold-standard FlowSOM (Aghaeepour, N. et al., 2013, Nature methods 10, 228-238). Across all 30 comparisons, CATCH significantly outperformed other comparisons in a statistically significant way (two-sided t-test between CATCH and each of the other clustering approaches, p-value<0.01) (FIG. 1A)

Automated Cluster Characterization and Earth Mover's Distance Between Genes in Synthetic and Real Single Cell Data

While manifold-intrinsic diffusion condensation implemented with an α-decay kernel can theoretically approximate ground truth cluster characterizations and compute differentially expressed genes, there was a desire to demonstrate this reasoning in synthetic and real single cell data. To empirically show that condensed transport approximates EMD between two clusters, EMD values were computed between genes using Wasserstein optimal transport as well as condensed transport on synthetic and real data using Gaussian and α-decay kernel implementations of diffusion condensation. Using single cell data generated from splatter, diffusion condensation were computed and the granularity with the highest topological persistence was identified using topological activity analysis. Ground truth and condensed transport differential expression values were computed by comparing every cluster at this granularity with every other cluster. In this analysis, a total of U.S. Pat. Nos. 12,130,200 and 4,535,640 gene comparisons were computed using Gaussian and α-decay approaches respectively. Comparing both Gaussian condensed transport and α-decay condensed transport values against ground truth per gene Wasserstein values, it was seen that the value in this α-decay approach (FIG. 2D) as it approximates ground truth Wasserstein distance with a correlation coefficient of 0.979. Furthermore, condensed transport computed all 4,535,640 gene comparison in 63 seconds while ground truth values were computed in 43,125 seconds, equating to a 684 fold increase in computational speed. This comparison in real single cell data was repeated, again comparing both approaches to ground truth Wasserstein EMD values, this time across 10 granularities identified by topological activity analysis. As previously performed, at each granularity, all clusters were compared to all other clusters using each approach. Across all comparisons, a total of U.S. Pat. Nos. 10,166,640 and 2,541,660 comparisons were computed for the Gaussian and α-decay implementations respectively. Again it was seen that α-decay is critical to accurately capturing ground truth EMD values, with α-decay condensed transport correlating highly with ground truth EMD while Gaussian condensed transport was less correlated (FIG. 1F). Furthermore, it was seen again that there is an increase in computational speed with this condensation based approach. In this weighted implementation, it is able to compute all 2,541,660 comparisons in 32 seconds, while ground truth EMD values were computed in 27,517 seconds, equating to a similar 860 fold increase in computational speed. Next, it was shown that this correlation between ground truth EMD and condensed 312 transport is not a feature of cluster granularity as defined by number of cluster (FIG. 1D). Finally, α-decay and Gaussian implementations were used to compute and compare cluster characterizations to ground truth in real single cell data. Using the same set of clusters and granularities as previously computed, it was shown that α-decay kernel again more accurately characterizes clusters than a Gaussian kernel (FIG. 1C).

CATCH Identifies the Complex Hierarchy of Cell Types and Cell States in AMD and Control Retinas.

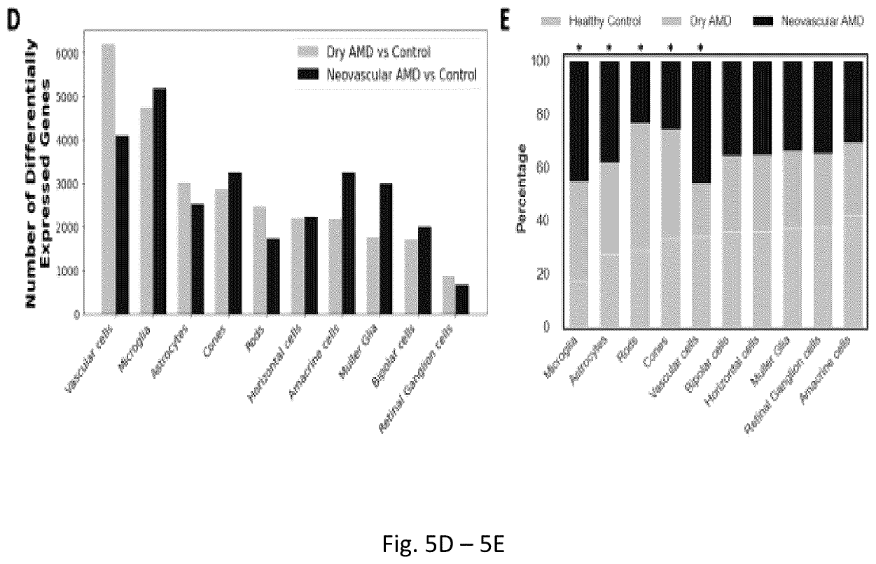

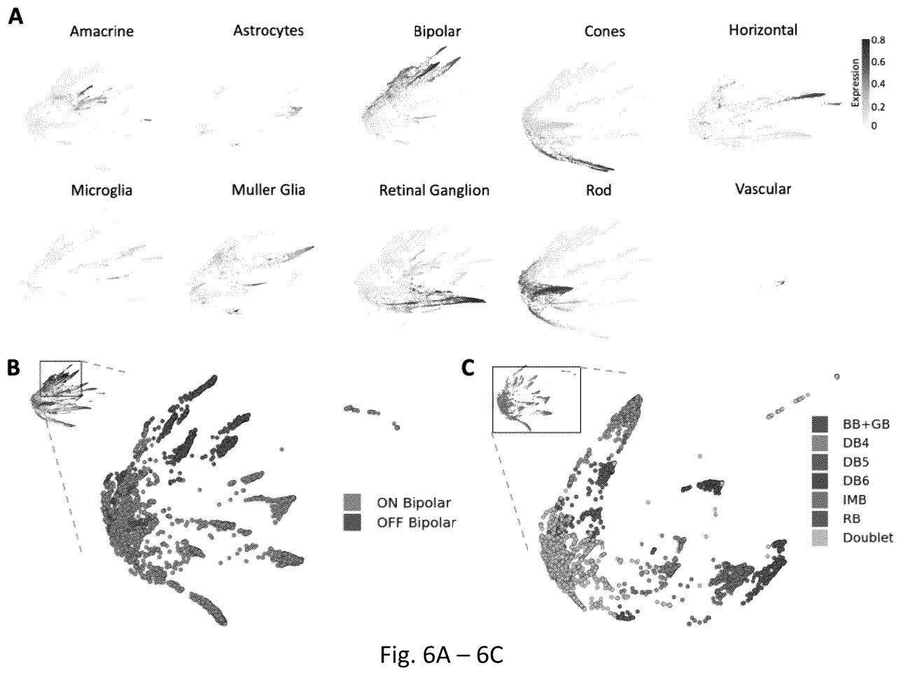

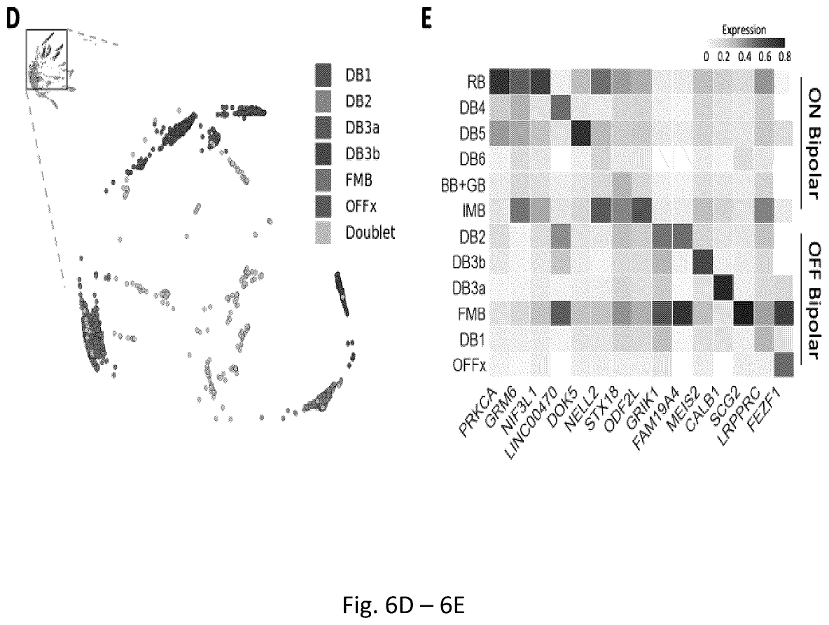

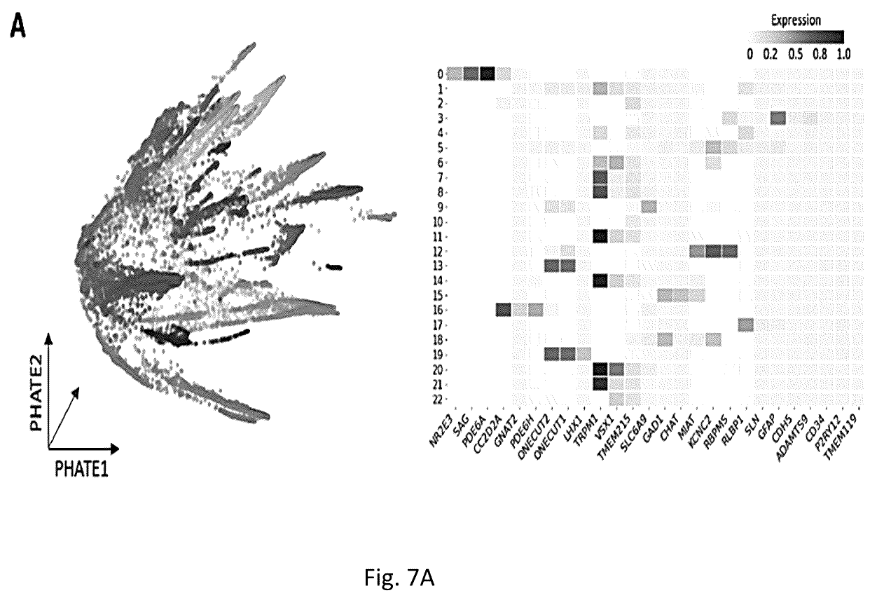

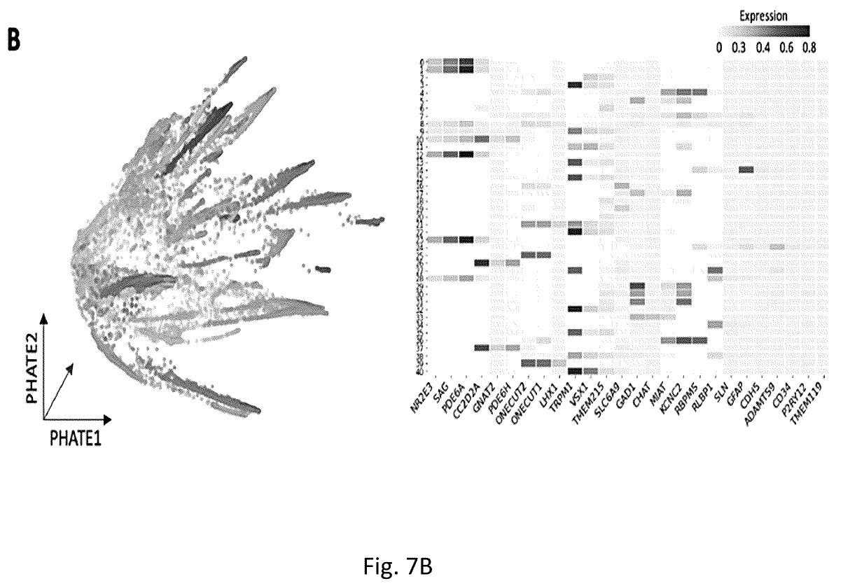

The retina is a complex central nervous system (CNS) tissue with many cell types and subtypes which disease status can differentially affect at various levels of granularity (FIG. 1A). As a component of the CNS, the retina shares features with the brain at the level of cell biology and degenerative pathology, including accumulation of extracellular material, e.g. drusen in AMD, (3-amyloid in AD, and myelin debris in MS (FIG. 1B). MS and AD, similar to AMD, have defined disease stages, each with an early or acute active phase, and a late or chronic phase (Faissner, S et al., 2019, Nat Rev Drug Discov 18, 905-922; Huang, W.-J et al., 2017, Exp. Ther. Med. 13, 3163-3166; Braak, H et al., 1991, Acta Neuropathol 82, 239-259). To identify cell populations across the cellular hierarchy that drive AMD progression through different stages, frozen retinal nuclei were isolated from the macula of lesion and non-lesion control samples and single-nucleus RNA-sequencing (snRNA-seq) was performed on 11 AMD tissue samples and 6 control tissue samples, creating the first single-cell dataset of AMD pathology. CATCH was then used to parse this dataset into meaningful groupings of cell types and states to understand the pathogenic mechanisms of disease. First, CATCH was applied to the AMD snRNA-seq dataset to identify the major cell types present in the healthy control and AMD samples. The persistence of each cluster in diffusion condensation was visualized through a barcode visualization, seeing a wide difference in overall cluster persistence (FIG. 5A). Next, topological activity analysis was performed which identified six granularities with high activity and high persistence. The snRNA-seq dataset was visualized using PHATE and the CATCH clusters were visualized at the coarsest two identified granularities (FIG. 5B). When visualizing the third granularity, a number of clusters were found isolated geometrically on the data manifold. Each of the unbiased populations at this granularities were categorized into cell types based on the expression of previously established cell type specific marker genes (FIG. 6A) (Menon, M. et al., 2019, Nat. Commun. 10, 4902). Using this approach, all neuronal cell types were identified, including retinal ganglion cells, horizontal cells, bipolar cells, rod photoreceptors, cone photoreceptors, and amacrine cells, as well as all rare non-neuronal cell types, including microglia, astrocytes, Müller glia, and vascular cells (FIG. 5C-D). To determine if these populations could be found with established approaches, Louvain23 clustering was applied to the AMD single-cell data. Louvain revealed 22 populations at coarse granularity, and 40 populations at fine granularity (FIG. 7A, B). Across both resolutions, however, rare innate immune cell types such as microglia, astrocytes and Müller glia, were not identified with the Louvain method, with markers specific for these cell types not localizing to any one cluster. Finally, to demonstrate the ability of CATCH to identify meaningful populations of cells across granularities, subtypes of bipolar cells, a diverse set of interneurons that transmits signals from rod and cone photoreceptors to retinal ganglion cells, were further explored (Peng, Y.-R. et al., 2019, Cell 176, 1222-1237.e22; Shekhar, K. et al., 2016, Cell 166, 1308-1323.e30; Yan, W. et al., 2020, Sci. Rep. 10, 9802). Analyzing a more fine grained granularity identified by topological activity analysis, the first two major subtypes of bipolar cells were identified, ON-center and OFF-center, which were marked by GRM6 and GRIK1, corresponding to the cells that depolarize and hyperpolarize, respectively, in response to light in their receptive field centers (FIG. 6B). By analyzing the next most persistent and active granularity within bipolar cells, all 12 major subtypes of cells were identified based on the expression of cell subtype specific marker genes (FIG. 6C-E). To identify cell types implicated in AMD pathogenesis in an unbiased manner, differential expression analysis was applied to the CATCH-identified cell types. By comparing the cells that originated from retinas with either dry or neovascular AMD to the cells from control retinas, gene expression differences were computed with earth mover's distance within each cell type (van Dijk, D. et al., 2018, Cell 174, 716-729.e27). By analyzing the number of differentially expressed genes across all cell types, it was found that vascular cells, microglia, and astrocytes had the greatest number of differentially expressed genes between both phases of AMD and control samples (FIG. 5E). Furthermore, abundance analysis was performed to identify if certain cell types were significantly more enriched in either dry or neovascular AMD. This analysis revealed a statistically significant increase in the proportion of microglia and astrocyte nuclei from donors with both dry and neovascular AMD compared to control samples (two-sided multinomial test, p-value <0.01) (FIG. 5F). Furthermore, there was a statistically significant enrichment of vascular cells in neovascular AMD, highlighting the importance of vascular cells in the development of pathological angiogenesis present at that stage of disease (two-sided multinomial test, p-value <0.01). Simultaneously, there was a significant depletion of both rod and cone photoreceptors in advanced neovascular AMD, corroborating the clinical understanding of the disease (two-sided multinomial test, p-value <0.01). These findings suggest that non-neuronal cell types including microglia, astrocytes, and vascular cells are important cell types across differential stages of AMD pathogenesis, with not only the most disrupted transcriptome but also significant enrichment in disease states.

Microglial Activation Signature Identified in Dry AMD is Shared Across the Early Phase of Multiple Neurodegenerative Diseases

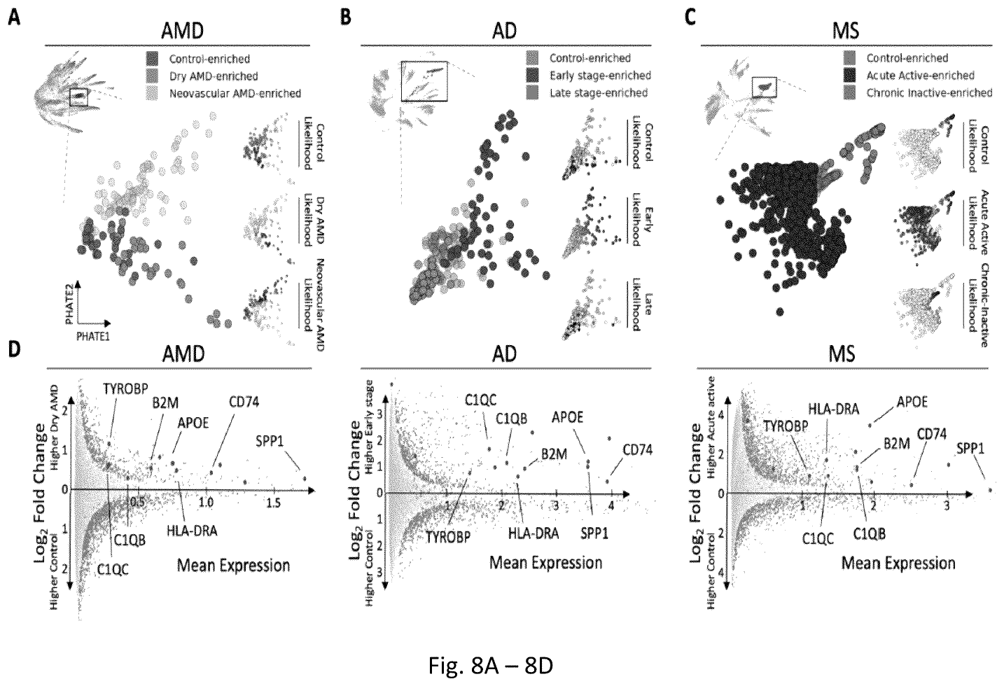

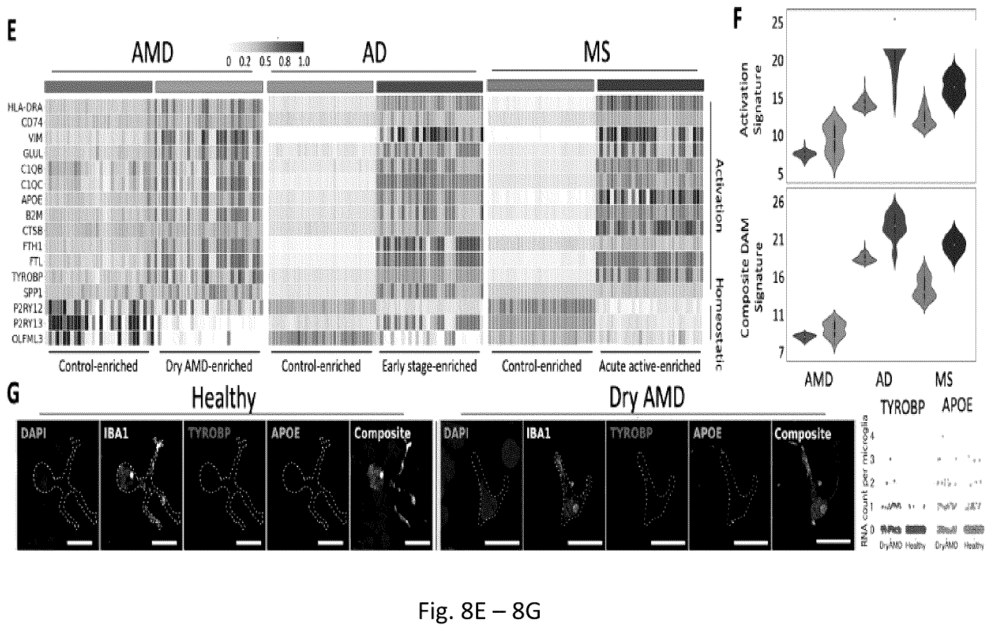

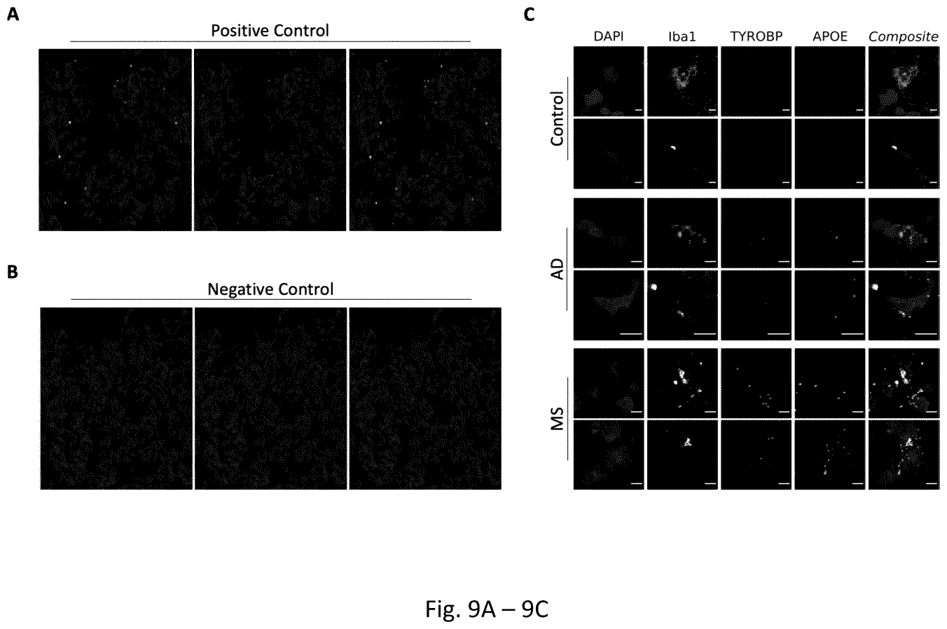

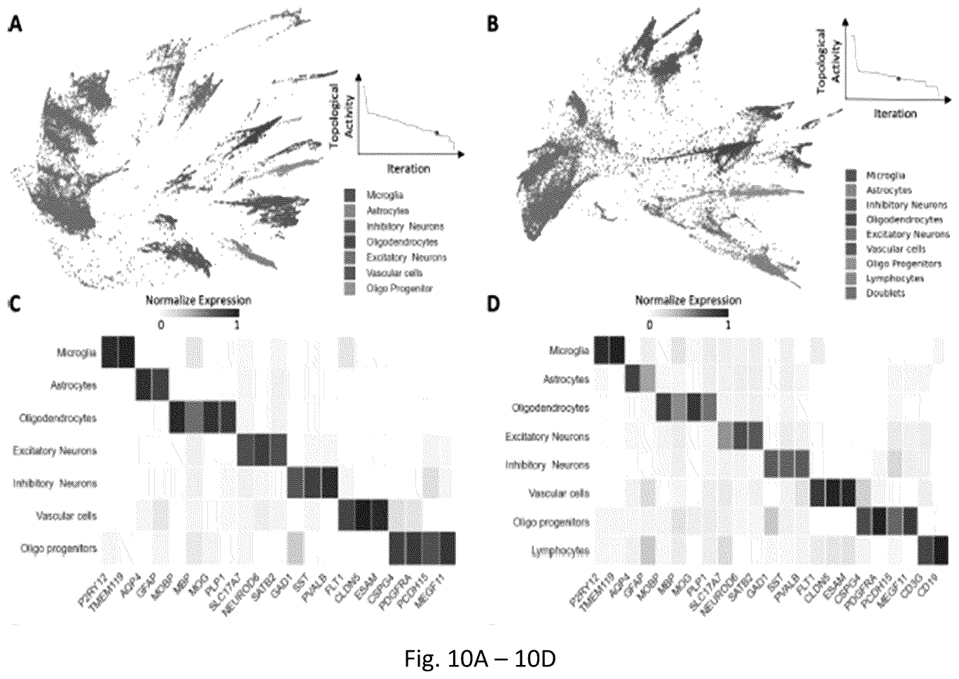

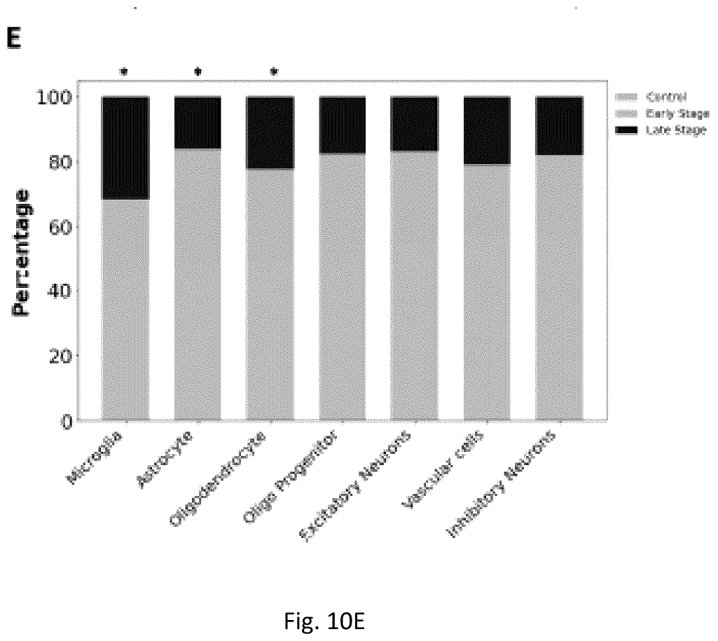

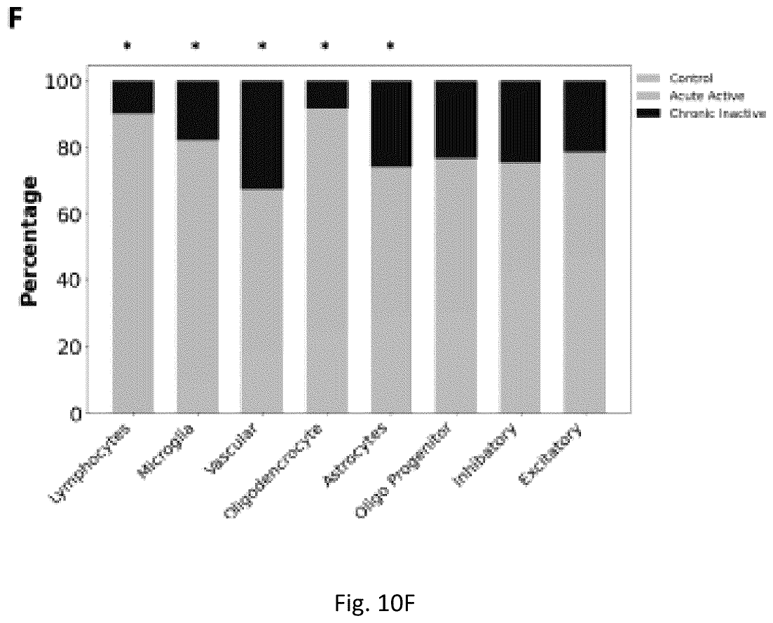

While microglia activation states and their dynamics have been identified in mouse models of AD and related to expression states found in humans, it is unclear how similar these states are and if their dynamics over the stages of degeneration remain the same (Keren-Shaul, H. et al., 2017, Cell 169, 1276-1290; Srinivasan, K. et al., 2020, Cell Reports 31, 107843). Furthermore, it is unclear to what extent these states and dynamics are shared across other neurodegenerative disease, like AMD. To date, the study of microglia in the CNS has been difficult due to their rarity, requiring focused enrichment strategies to thoroughly study (Keren-Shaul, H. et al., 2017, Cell 169, 1276-1290; Srinivasan, K. et al., 2020, Cell Reports 31, 107843). Fine grained analysis is further confounded in the retina as microglia are the rarest cell type, making fine grained analysis on this cell type impossible to perform. With the ability of CATCH to sweep across all hierarchies of clusters; however, it is easy to identify subpopulations of even the rarest cell types at fine granularity to perform a rigorous and in-depth analysis of cellular states. To identify microglial subpopulations enriched in specific disease phases of AMD and build transcriptomic signatures of disease, CATCH granularities were identified that isolated high MELD likelihood scores computed for control, dry, and neovascular AMD conditions. Using this computational approach, MELD likelihood scores for each condition on all microglia in AMD were computed (FIG. 8A). Next, granularities identified by this topological activity analysis were searched to identify a set of clusters that partitioned regions of high disease likelihood from regions of low likelihood. With this approach three clusters were identified, each enriched for a different condition: a cluster enriched for cells from control samples, a cluster enriched for cells from early, dry AMD samples, and a cluster enriched for cells from late-stage, neovascular AMD samples (FIG. 8A). To identify signatures of AMD present in microglia during the early stage of disease pathogenesis, a phase in which microglia have been previously implicated (Mitchell, P. et al., 2018, Lancet 392, 1147-1159), condensed transport was performed between the gene expression landscapes in the control-enriched and the dry AMD-enriched clusters. Analyzing the top 100 most differentially expressed genes between these subpopulations, a clear activation signature appeared in the early, dry AMD-enriched cluster, including APOE, TYROBP, and SPP1 (FIG. 8D), genes known to play a role in neurodegeneration (Keren-Shaul, H. et al., 2017, Cell 169, 1276-1290). The association of TYROBP and APOE were validated on sections of human retinal macula by simultaneous immunofluorescence for IBA1, a microglia-associated gene, and in situ hybridization for TYROBP and APOE. On sections of human retinal macula, MA1-positive cells from patients with dry AMD showed enrichment relative to controls for gene transcripts from TYROBP and APOE, indicating polarization of a subset of microglia towards the neurodegenerative microglial phenotype in early disease (FIG. 8G). Increased expression of TYROBP and APOE in microglia was also identified using in situ hybridization on lesions from human brain tissue with early stage AD and early progressive MS compared with controls (FIG. 9C). Due to the similarity between this activation state and a previously defined disease-associated microglial state described in mice (Keren-Shaul, H. et al., 2017, Cell 169, 1276-1290; Friedman, B. A. et al., 2018, Cell Rep 22, 832-847), a comprehensive analysis of microglial states in two other neurodegenerative diseases, AD and progressive MS, was performed. Applying this CATCH approach to snRNA-seq data from AD and MS, all major cell types were identified based on the expression of cell type specific marker genes (FIG. 10A-D) (Mathys, H. et al., 2019, Nature 570, 332-337; Schirmer, L. et al., 2019, Nature 573, 75-82). As in AMD, enrichment analysis revealed that microglia were significantly enriched in AD and MS when compared to control brain tissue (FIG. 10E-F). Similar to this analysis of AMD identifying disease-phase specific transcriptomic states, MELD and topological activity analysis were applied to microglia in the AD and MS datasets and three clusters of microglia were identified in each disease: a cluster enriched for cells from control brain tissue; a cluster enriched for cells from early-stage AD tissue or acute active MS lesions; and a cluster enriched for cells from late stage AD tissue or chronic inactive MS lesions (FIG. 8B,C). Condensed transport differential expression analysis between the control-enriched and the early disease-enriched clusters yielded a common shared activation profile in all three diseases when analyzing the top 10% of differentially expressed genes (FIG. 8D, middle and right panels). Compared with the mixed results of other single cell studies of shared signatures of microglial activation across disease (Mathys, H. et al., 2019, Nature 570, 332-337; Thrupp, N. et al., 2020, Cell Rep 32, 108189; Zhou, Y. et al., 2020, Nat Med 26, 131-142; Deczkowska, A. et al., 2018, Cell 173, 1073-1081), the successful detection in the present study of human tissues lies in the sensitivity derived from the CATCH method's ability to analyze cells from various tissues that fall within the same granularity in their respective hierarchies. To understand the early disease enriched microglial populations, the microglial activation signature was visualized (CD74, SPP1, VIM, FTL, B2M) (APOE, TYROBP, CTSB) (C1QB and C1QC) as well a homeostatic signature (P2RY12, P2RY13, and OLFML3) on control-enriched and early disease-enriched clusters from all three degenerative conditions (FIG. 8E). Across conditions, a clear divergence is seen between the expression pattern of this homeostatic signature in control-enriched populations and early disease-enriched populations across conditions. With higher expression of activation genes and lower expression of homeostatic genes, it is clear that these early activated populations display a divergent polarization state. A composite microglial activation signature was built and mapped onto these clusters along with a previously described disease-associated microglia signature found in an AD mouse model (Keren-Shaul, H. et al., 2017, Cell 169, 1276-1290). The early disease-enriched clusters displayed higher expression of both signatures compared with control-enriched clusters (FIG. 8F). This shared neurodegenerative microglial phenotype across AMD, MS, and AD involves upregulation of multiple genes implicated in studies of neurodegenerative disease risk. These include APOE, a key regulator of the transition between homeostatic and neurotoxic states in microglia strongly implicated in risk for AD and AMD; TYROBP which encodes the TREM2 adaptor protein DAP12, mutations of which are implicated in a frontal lobe syndrome with AD-like pathology and expression of which is upregulated in white matter microglia in MS lesions; SPP1 (osteopontin), implicated in microglial activation in brains affected by MS and AD; and CTSB, encoding the major protease in lysosomes cathepsin-B, which is upregulated in microglia responding to (3-amyloid plaques in AD (Krasemann, S. et al., 2017, Immunity 47, 566-581; Corder, E. H. et al., 1993, Science 261, 921-923; Lambert, J. C. et al., 2013, Nat Genet 45, 1452-1458; Fritsche, L. G. et al., 2016, Nat Genet 48, 134-143; Satoh, J. I et al., 2018, Intractable Rare Dis Res 7, 32-36; van der Poel, M. et al., 2019, Nature Communications 10, 1139; Sala Frigerio, C. et al., 2019, Cell Rep 27, 1293-1306). Initiation of the pathologic accumulation of extracellular material occurs by different means in these three neurodegenerative diseases. However, the finding that microglial phagocytic, lipid metabolism, and lysosomal activation pathways are upregulated in the early or acute active stage of all three diseases suggests a convergent role for dysregulation in microglia directed towards clearance of extracellular deposits of debris.

Astrocyte Activation Signature Identified in Dry AMD is Shared Across the Early Phase of Multiple Neurodegenerative Diseases

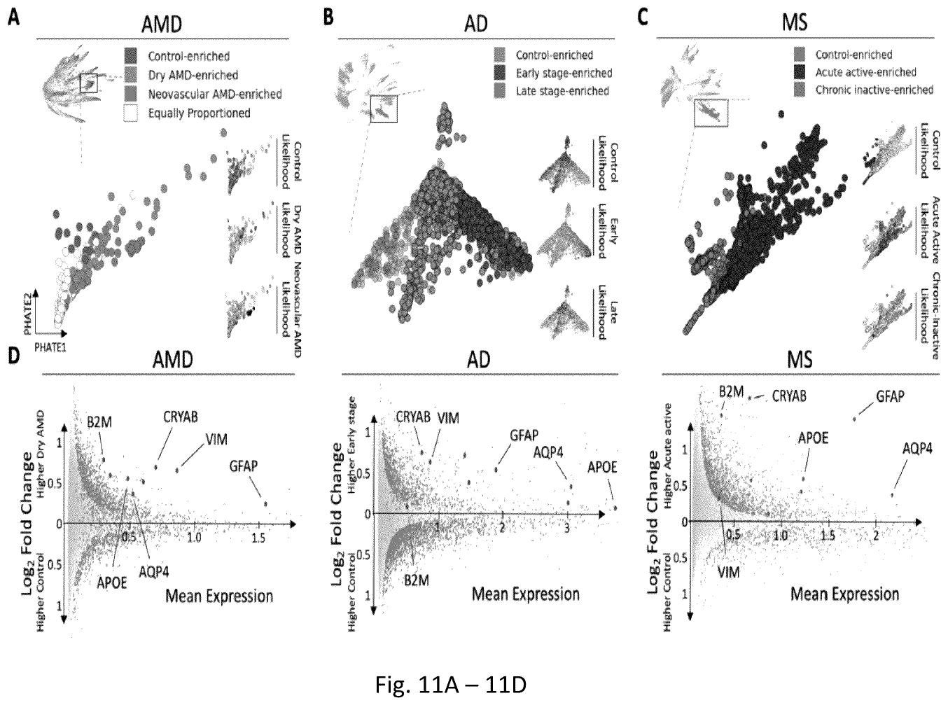

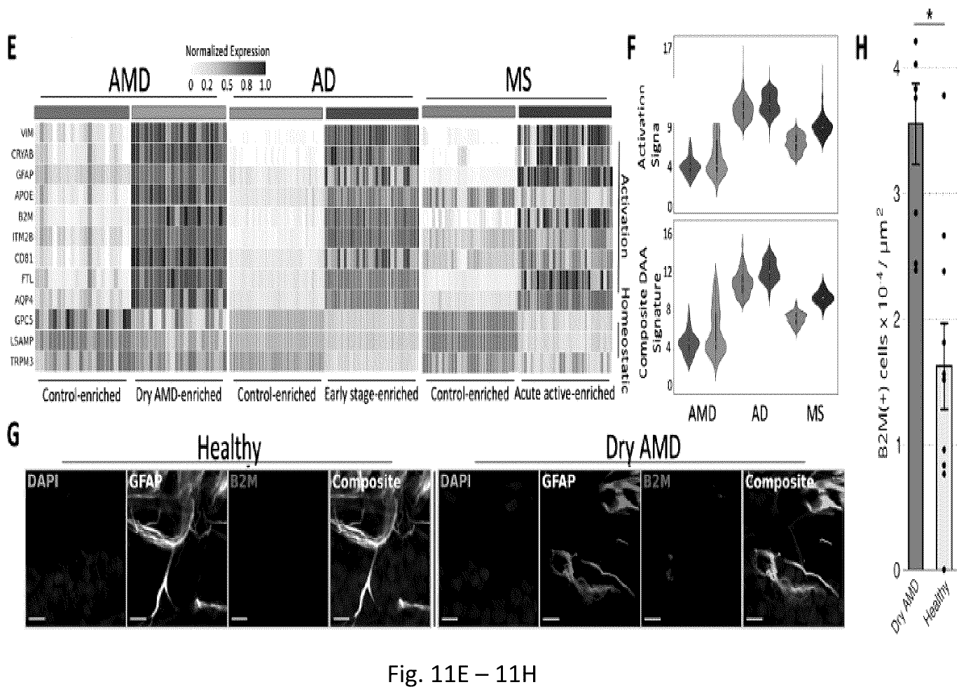

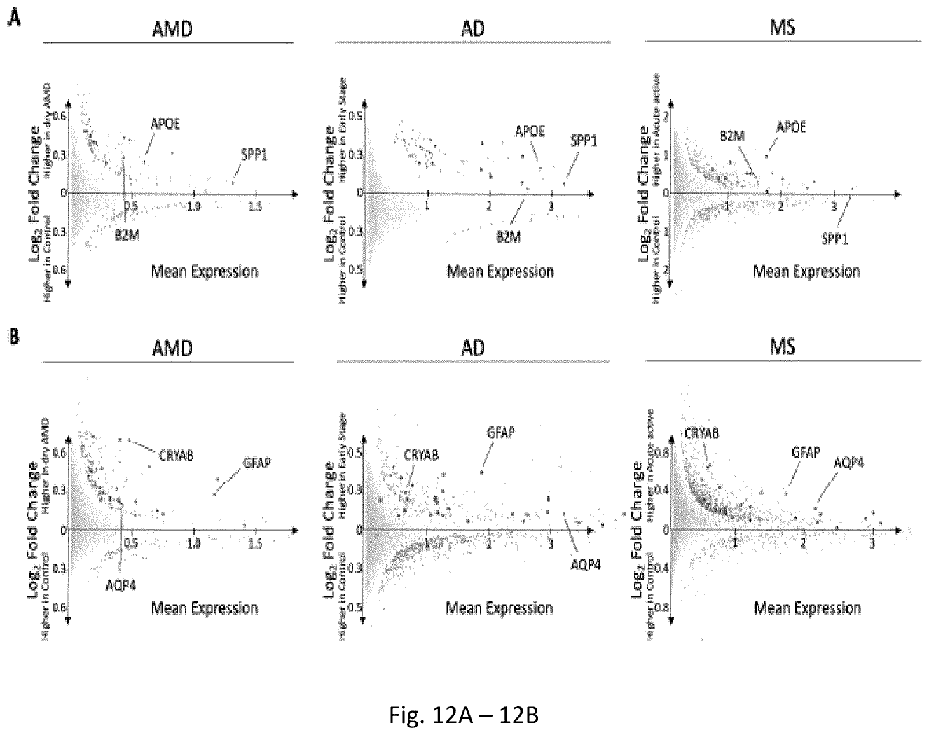

While astrocyte transcriptomic states and dynamics have been established in mouse models of AD, due to disease dynamics and their rarity, astrocyte profiles have not been characterized in human AMD (Habib, N. et al., 2020, Nat Neurosci 23, 701-706). Because initial analysis implicated astrocytes in disease pathogenesis (FIG. 5E-F), similar cross-disease analysis was performed within the astrocyte populations using CATCH. Using MELD and topological activity analysis, four clusters of astrocytes were identified at a finer granularity within the diffusion condensation hierarchy: a cluster enriched for cells from control samples, a cluster enriched for cells from patients with early, dry AMD, a cluster enriched for cells from patients with late-stage neovascular AMD and a cluster with equal numbers of cells from all three conditions (FIG. 11A). When comparing the transcriptomic profiles of cells within the dry AMD-enriched and control-enriched astrocyte populations using condensed transport, key activation and degeneration associated genes, such as GFAP, VIM, and B2M were upregulated (FIG. 11D), highlighting the need for a cross disease comparison of the astrocytes. Using MELD and topological activity analysis, clusters were identified that isolated phase specific populations within MS and AD astrocytes. In both diseases, three clusters were identified: a cluster enriched for cells from control brain tissue, a cluster enriched for cells from early-stage AD tissue or acute active MS lesions, and a cluster enriched for cells from late-stage AD tissue or chronic inactive MS lesions (FIG. 11B,C). By comparing the control-enriched and early disease-enriched clusters within each dataset using condensed transport, a shared gene signature enriched in the early disease subcluster across all three diseases was identified (FIG. 11E). The integrated gene signature included markers of activated astrocytes, including VIM, GFAP, CRYAB, and CD81, major histocompatibility complex (MHC) class I (B2M), iron metabolism (FTH1 and FTL), a water channel component implicated in debris clearance (AQP4), along with lysosomal activation and lipid and amyloid phagocytosis (CTSB, APOE) (Giovannoni, F. et al., 2020, Trends Immunol 41, 805-819; Zamanian, J. L. et al., 2012, J Neurosci 32, 6391-6410; Bombeiro, A. L et al., 2017, Neurosci Lett 647, 97-103; Ransohoff, R. M. et al., 1991, Arch Neurol 48, 1244-1246; Xie, L. et al., 2013, Science 342, 373-377). Of interest, many upregulated genes were shared between the microglial and astrocyte early activation signatures, suggesting common glial stress pathways may become activated in response to neurodegeneration. Similar to microglia, homeostatic (GPC5, LSAMP, TRPM3) and composite activation signatures (B2M, CRYAB, VIM, GFAP, AQP4, APOE, ITM2B, CD81, FTL) were mapped to early disease-enriched and control-enriched astrocyte clusters across degenerative conditions. Similar to microglia, the composite activation signature and homeostatic signatures were divergently expressed by these early enriched clusters (FIG. 11E,F, upper). Using a recently published disease-associated astrocyte signature established in an AD mouse model (Habib, N. et al., 2020, Nat Neurosci 23, 701-706), a composite activation signature was built and mapped onto the early-disease and control-enriched clusters across conditions. It was identified that the early disease-enriched clusters displayed higher expression of the disease-associated astrocyte gene signature in addition to the composite activation signature (FIG. 11F, lower). To validate the astrocyte signature in tissue, simultaneous GFAP immunofluorescence and RNA in situ hybridization was performed for B2M, a component of MHC-I and member of the shared gene signature, on sections of human macula. The retinal layers occupied by GFAP-positive astrocytes (inner plexiform layer to inner limiting membrane) contained a higher density of B2M transcripts in retinas affected by dry AMD relative to control retina (p-value=<1e-03, two-sided Student's t-test) (FIG. 11G,H).

CATCH Computes Robust Shared Disease Signatures from Data

Historically, shared cell type specific pathogenic gene signatures have been challenging to build due to the heterogeneity present in disease states. Previous approaches that have tried to explicitly build degenerative signatures in astrocytes and microglia specifically isolated these cell types from mice, biasing their analysis (Habib, N. et al., 2020, Nat Neurosci 23, 701-706; Keren-Shaul, H. et al., 2017, Cell 169, 1276-1290). A similar enrichment approach in human AD identified a related microglial signature (Srinivasan, K. et al., 2020, Cell Reports 31, 107843). These biased analysis approach excluded the ability to study other cell types in a rigorous manner, a problem as most single cell studies are explorative and do not want to target a specific cell type a priori. The multigranular approach of this invention not only robustly translated degenerative signatures found in mice to humans, but showed their shared nature across different diseases in multiple cell types. This is largely due to the multigranular and MELD-based computational enrichment based approach to identifying cellular states enriched in different disease phases. To illustrate this point, microglia and astrocytes from each of the degenerative datasets were taken and differentially expressed genes between glia originating from healthy samples and glia originating from early disease affected samples were computed. Comparing the most enriched genes from each of these comparisons, significantly fewer genes were found that are shared. Further, in astrocytes and microglia only three genes that were identified in previously described shared signatures were found (FIG. 12). This reveals the value of CATCH and multigranular analysis as studying populations of cells at the non-optimal granularity of cell type produces weak gene signatures that cannot be generalized. Identifying meaningful subpopulations of cells enriched in different disease settings can produce robust and translatable signatures.

Studying Shared Human Glial Landscape Disease Dynamics with CATCH

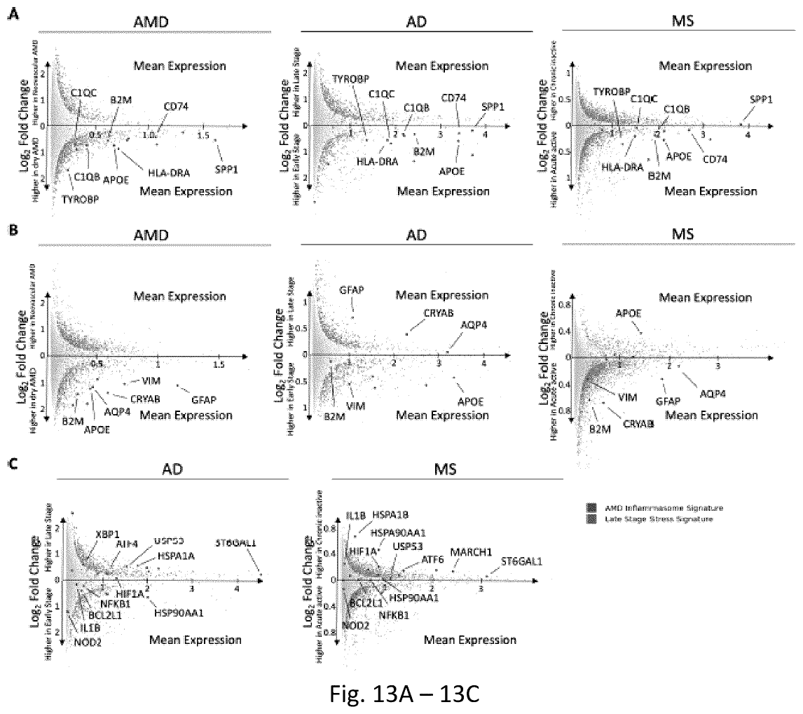

Previously established glial degenerative signatures in mouse have attempted to characterize the dynamics over the progression of disease from early to late phases. It has been reported that microglia transition from an early degenerative signature to a late degenerative signature through TREM2 and astrocytes increase expression of their disease associated signature during disease progression (Keren-Shaul, H. et al., 2017, Cell 169, 1276-1290; Habib, N. et al., 2020, Nat Neurosci 23, 701-706). While these dynamics were established in mouse models of the disease, the disease phase specificity of these states in humans remains unknown. To understand glial activation dynamics across AMD, AD and MS in humans, condensed transport between early disease and late disease-enriched clusters of astrocytes and microglia was performed. Across both comparisons, it was seen that signatures present in the early phase of human AMD, MS, and AD are not detected in microglia and astrocytes during the late stage of human disease (FIG. 13A-B).

Microglia Display Inflammasome Activation Signature and Astrocytes Display Pro-Angiogenic Signature in Late Stage AMD

Glial dynamics may contribute to an inflammatory and damaging response driving AMD disease progression. To test this, glial transcriptomic states enriched in late neovascular AMD where degeneration is a more prominent feature were identified. To identify glial signatures in late stage neovascular AMD, snRNAseq was performed on three additional retinas from human donor retinas with neovascular AMD, and applied CATCH to the resulting total 46,783 nuclei when combined with the previously sequenced samples. As done previously, a granularity of the CATCH hierarchy with low topological activity was identified and cell type labels were assigned based on the expression of cell type-specific gene signatures (FIG. 14A,B). Following the fine grained CATCH analysis done previously, two clusters of microglia were identified: one cluster enriched for cells from control retinal samples and one cluster enriched for cells from late-stage, neovascular AMD retinal samples (FIG. 14C). To identify signatures of AMD present in the subpopulation of microglia that were enriched in the late stage of disease, condensed transport between the gene expression landscapes of the control-enriched and the neovascular AMD-enriched clusters was performed. Analyzing the top 10% of differentially expressed genes between these subpopulations revealed an inflammasome-related signature including IL1B, NOD2, and NFKB1. The pro-IL-1β protein requires both cleavage and release via inflammasome-mediated caspase activation and pyroptosis for bioactivity (Latz, E et al., 2013, Nature Reviews Immunology 13, 397-411). Here, activation of inflammasome sensors and oligomerization into proteolytically active complexes may occur in response to a significant and lasting drop in oxygen tension or chronic lipid exposure (Latz, E et al., 2013, Nature Reviews Immunology 13, 397-411; Cantuti-Castelvetri, L. et al., 2018, Science 359, 684-688), both known to drive inflammasome activation via NLRP3 (NOD-, LRR- and pyrin domain-containing 3) (FIG. 14D). However, 520 this inflammasome activation signature was not present in microglia of late stage AD and MS (FIG. 13C). Instead, alternative cellular stress-associated pathways were upregulated including transcriptional regulators of the ER stress response (XBP1) and their target genes involved in protein folding and transport (HSPA1A, HSPA1B, HSP90AA1) and glycosylation (ST6GAL1 and ST6GALNAC3), as well as regulators of autophagy and proteostasis (ATG7, MARCH1, USP53). These signatures highlight a shared cellular stress induction in the neurodegenerative microenvironment, which may engage disease-specific programs such as inflammasome activation and pyroptosis in AMD that ultimately drive various components of disease progression. Using the fine grained CATCH work flow, two astrocyte subpopulations were identified: one cluster enriched for cells from control retinal samples and one cluster enriched for cells from late-stage, neovascular AMD retinal samples (FIG. 14C). To identify signatures of AMD present in astrocytes during the late stage of disease pathogenesis, condensed transport between the genes in the control-enriched and the neovascular AMD-enriched clusters was performed. Analyzing the top 10% of differentially expressed genes between these subpopulations revealed elevation of VEGFA, NR2E1, and HIF1A expression (FIG. 14D), all of which are regulators of cellular responses to low oxygen tension (Shweiki, D et al., 1992, Nature 359, 843-845; Zeng, Z. J. et al., 2012, Biol Open 1, 527-535; Wang, G. L et al., 1995, Proc Natl Acad Sci USA 92, 5510-5514). While VEGFA is known to be an important mediator of the abnormal blood vessel growth that characterizes late-stage neovascular AMD and is the target of current therapies for the treatment of disease (Kliffen, M et al., 1997, Br J Ophthalmol 81, 154-162; Wong, T. Y et al., 2007, Lancet 370, 204-206; Fritsche, L. G. et al., 2016, Nat Genet 48, 134-143), these data provide the first demonstration in humans of a specific subpopulation of retinal astrocytes that are a source of this signal.

Microglia-Derived IL-1β Drives Pathologic Neovascularization Via Astrocytes

As microglia are known to polarize astrocyte functional states through the secretion of soluble factors, it was determined if any microglia derived cytokines could drive VEGFA expression from retinal astrocytes (Escartin, C. et al., 2021, Nat Neurosci 24, 312-325; Guttenplan, K. A. et al., 2020, Cell Rep 31, 107776; Liddelow, S. A. et al., 2017, Nature 541, 481-487). Since CATCH was able to isolate astrocyte and microglial states, CellPhoneDB interaction analysis was utilized to create a putative list of possible microglia-derived cytokines that may interact with astrocytes to drive VEGFA expression (FIG. 15A) (Efremova, M et al., 2020, Nature Protocols 15, 1484-1506). From this analysis, the neovascular-enriched microglia cluster interacted most significantly with astrocytes through IL-1β, IL-6, while in controls, microglia-astrocyte interaction was primarily mediated by IL-4. Furthermore, IL-1β interacted most significantly with the neovascular-enriched astrocyte subpopulation. Using DREMI, it was found that IL-1β signaling on astrocytes was most significantly associated with astrocyte production of VEGFA (Krishnaswamy, S. et al., 2014, Science 346, 1250689-1250689). Meanwhile IL-4 signaling was most significantly associated with a decrease in astrocyte VEGFA production (FIG. 15B). The cytokine regulators of astrocyte VEGFA production were then validated in an unbiased manner. Cytokines are a part of a complex network of proteins that can produce additive, synergistic, or antagonistic effects. A combinatorial screening approach was used utilizing all cytokines identified in the snRNAseq dataset, removing one at a time to test its necessity in creating a VEGFA expressing astrocyte. Screening with human-iPSC-derived astrocytes demonstrated that IL-1β, IL-1β and IL-17 are positive regulators of VEGFA production in these cells as their subtraction causes decreased VEGFA compared to astrocytes stimulated with all cytokines) (FIG. 15C, y axis). The sufficiency of some of these cytokines being able to regulate VEGFA production was then tested by completing a single protein stimulation and it was noted that, interestingly, only IL-1β caused astrocyte VEGFA secretion (FIG. 15C, x axis). Across all analyses, only IL-1β positively regulated induction of VEGFA from astrocytes both in vitro (FIG. 15C) and in silico (FIG. 15B). Meanwhile, this analysis of VEGFA regulation also validated the computational prediction of IL-4 being a negative regulator of VEGF-A production (FIG. 15B-C), showing the utility of this approach in identifying signaling interactions between cellular subsets identified with CATCH. With identification of cytokine mediators of astrocyte VEGFA production, the in vivo findings were validated by injecting IL-1β intravitreally in a mouse. This resulted in upregulation of VEGFA (FIGS. 15D and E). Not only was there an increase in the amount of VEGFA (FIG. 15F, right), there was an increase of overlapping signals of GFAP and VEGFA, indicative of astrocyte VEGFA activation and secretion (FIG. 15F, left), along with VEGFA expression extending from ganglion cell layer localization down to other layers of the retina. Altogether, this demonstrated the sufficiency of cytokines such as IL-1β to induce VEGFA secretion in astrocytes in vitro and in vivo. Cytokines such as IL-1β are increased in the vitreous of patients with neovascular AMD, but source and the role of these cytokines in angiogenesis has not been explored (Zhao, M. et al., 2015, PLoS One 10, e0125150). Immunohistochemical staining for IL-1β in retinal samples from the macula of patients with AMD and healthy controls were done, and an increased amount of IL-1β intensity was observed in the inner retinal layers, where astrocytes reside. Furthermore, upregulation of VEGFA was seen in these areas (FIG. 15G), indicating that the phenomenon observed in vitro and in mice likely occurs in neovascular AMD patients as well (FIG. 15F-H).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A system for detecting at least one cell population or biomarker, the system comprising:
a non-transitory computer-readable medium with instructions stored thereon, which when executed by a processor perform steps comprising:
collecting a quantity of cellular data;
providing a CATCH toolkit, wherein the CATCH toolkit comprises a set of topologically inspired machine learning tools to identify, characterize and compare populations of cells across the cellular hierarchy;
providing the cellular data to the CATCH toolkit; and
calculating the level of at least one cellular population with the CATCH toolkit from the cellular data.

2. The system of claim 1, wherein the CATCH toolkit comprises a set of machine learning tools for:
a) determination of persistent homology;
b) a topologically-inspired approach to understand the multigranular structure of single cells based on their inherent manifold geometry;
c) diffusion condensation; and
d) differential expression analysis via approximation of Wasserstein earth mover's distance.

3. The system of claim 2, wherein the method of diffusion condensation comprises:
a) dynamically learning the geometry of the single cell manifold with each diffusion filter using spectral entropy;
b) visualizing learned topology via embedding of condensation homology;
c) use of the topological activity to identify meaningful granularities for downstream analysis;
d) implementing diffusion operator landmarking, weighted random walks and data merging to efficiently scale to thousands of cells; and
e) implementing diffusion condensation with alpha decay kernel for automated cluster characterization and efficient computation of differentially expression genes with condensed transport.

4. The system of claim 1, wherein the cellular data is single cell datasets.

5. The system of claim 1, wherein the cellular data is snRNAseq data.

6. An assay for detecting at least one cellular population in a sample, the method comprising:
a) obtaining cellular data from a sample;
b) applying the cellular data to the system of claim 1, wherein the system comprises a CATCH toolkit, wherein the CATCH toolkit comprises a set of topologically inspired machine learning tools to identify, characterize and compare populations of cells across the cellular hierarchy; and
c) calculating the level of at least one cellular population with the CATCH toolkit from the cellular data.

7. The assay of claim 6, wherein the cellular data is single cell data.

8. The assay of claim 6, wherein the cellular data is snRNAseq data.

9. The assay of claim 6, wherein the sample is a biological sample.

10. The assay of claim 6, wherein the sample is a patient sample.

11. A method of diagnosing a disease or disorder associated with a rare cell population in a subject in need thereof, the method comprising
a) obtaining a sample from the subject;
b) obtaining cellular data from the sample;
c) applying the cellular data to the system of claim 1, wherein the system comprises a CATCH toolkit, wherein the CATCH toolkit comprises a set of topologically inspired machine learning tools to identify, characterize and compare populations of cells across the cellular hierarchy; and
d) calculating the level of at least one rare cell population with the CATCH toolkit from the cellular data;
e) comparing the level of at least one rare cell population detected in the patient sample to a comparator control level of the rare cell population;
f) diagnosing the subject as having or at risk of a disease or disorder when the level of at least one rare cell population detected in the patient sample is significantly increased or decreased relative to a predetermined cut-off or comparator control level of the rare cell population.

12. The method of claim 11, further comprising administering a treatment based on the diagnostic outcome for the disease or disorder.

13. A method of determining the prognosis of a disease or disorder in a subject in need thereof, the method comprising
a) obtaining a sample from the subject;
b) obtaining cellular data from the sample;
c) applying the cellular data to the system of claim 1, wherein the system comprises a CATCH toolkit, wherein the CATCH toolkit comprises a set of topologically inspired machine learning tools to identify, characterize and compare populations of cells across the cellular hierarchy; and
d) calculating the level of at least one rare cell population with the CATCH toolkit from the cellular data;
e) comparing the level of at least one rare cell population detected in the patient sample to a comparator control level of the rare cell population;
f) identifying the subject as having better or worse prognosis of a disease or disorder when the level of at least one rare cell population detected in the patient sample is significantly increased or decreased relative to a predetermined cut-off or comparator control level of the rare cell population.

14. The method of claim 13, further comprising administering a treatment based on the prognostic outcome for the disease or disorder.

15. A method of treating neovascular AMD, the method comprising administering an IL-1β inhibitor to a subject diagnosed with neovascular AMD.

16. The method of claim 15, wherein the IL-1β inhibitor is selected from the group consisting of a small interfering RNA (siRNA), a microRNA, an antisense nucleic acid, a ribozyme, an expression vector encoding a transdominant negative mutant, an antibody, a peptide, a chemical compound and a small molecule.

17. The method of claim 15, wherein the IL-1β inhibitor is targeted for delivery to microglia.
